(19) United States
(12) Patent Application Publication
(10) Pub. No.: US 2010/0113504 A1
(43) Pub. Date: May 6, 2010

(54) AMIDE COMPOUNDS AND THEIR USE AS ANTITUMOR AGENTS

(75) Inventors: Stephan Hanessian, Beaconsfield (CA); Luciana Auzzas, Sassari (IT); Giuseppe Giannini, Pomezia (IT); Claudio Pisano, Aprilia (IT); Loredana Vesci, Roma (IT); Walter Cabri, Rozzano (IT)

Correspondence Address: LUCAS & MERCANTI, LLP, 475 PARK AVENUE SOUTH, 15TH FLOOR, NEW YORK, NY, 10016, US

(73) Assignee: SIGMA-TAU INDUSTRIE FARMACEUTICHE RIUNITE S.P.A., Roma (IT)

(21) Appl. No.: 12/528,912

(22) PCT Filed: Mar. 12, 2008
(86) PCT No.: PCT/EP08/52965

§ 371 Date: Nov. 17, 2009

(30) Foreign Application Priority Data
Mar. 13, 2007 (EP) 07104071.1

(51) Int. Cl. A61K 31/4353; C07C 233/00; C07D 273/01; C07D 273/02; C07D 498/04; A61K 31/16; A61K 31/395; A61K 31/4162; A61K 31/407; A61P 35/04

(52) U.S. Cl. 514/293; 564/158; 540/455; 514/616; 514/450; 514/406; 514/411

(57) ABSTRACT

The present invention relates to a compound of Formula (I), its geometrical isomers, in an optically active form as enantiomers, diastereomers, as well as in the form of racemate, as well as pharmaceutically acceptable salts thereof, wherein R is selected from CONHOH, CONHCH₂SH, CONHCH₂SCOCH₃, SH, SCOCH₃, SCH₃, N(OH)COH, COCONHCH₃ and CF₃ for the preparation of a medicament, in particular for selectively inducing terminal differentiation of neoplastic cells and thereby inhibiting proliferation of such cells, for inducing differentiation of tumor cells in a tumor, for inhibiting the activity of histone deacetylase and for the treatment of primary cancer or secondary cancer.

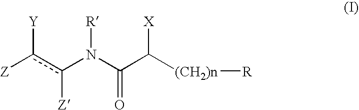

### TECHNICAL FIELD

The present invention relates to novel amide compounds and their use as anti-tumoral and pro-apoptotic agents.

### BACKGROUND ART

Cancer is a disorder in which a population of cells has become, in varying degrees, unresponsive to the control mechanisms which normally govern proliferation and differentiation. A recent approach to cancer therapy has been to attempt induction of terminal differentiation of the neoplastic cells (Sporn, M. B. et al. (1985) in Cancer: Principles and Practice of Oncology, eds. Hellman, S., Rosenberg, S. A., and DeVita, V. T., Jr., Ed. 2, (J. B. Lippincott, Philadelphia), P. 49). In cell culture models differentiation has been reported by exposure of cells to a variety of stimuli, including: cyclic AMP and retinoic acid (Breitman et al. Proc. Natl. Acad. Sci. USA 1980, 77, 2936-2940 and Olsson et al. Cancer Res. 1982, 42, 3924-3927), aclarubicin and other anthracyclines (Schwartz et al. Cancer Res. 1982, 42, 2651-2655).

There is abundant evidence that neoplastic transformation does not necessarily destroy the potential of cancer cells to differentiate. There are many examples of tumor cells which do not respond to the normal regulators of proliferation and appear to be blocked in the expression of their differentiation program, and yet can be induced to differentiate and cease replicating. A variety of agents, including some relatively simple polar compounds, derivatives of vitamin D and retinoic acid, steroid hormones, growth factors, proteases, tumor promoters, and inhibitors of DNA or RNA synthesis, can induce various transformed cell lines and primary human tumor explants to express more differentiated characteristics.

Some studies identified a series of polar compounds that were effective inducers of differentiation in a number of transformed cell lines. One such effective inducer was the hybrid polar/apolar compound N,N′-hexamethylene bisacetamide (HMBA), another was suberoylanilide hydroxamic acid (SAHA). The use of these compounds to induce murine erythroleukemia (MEL) cells to undergo erythroid differentiation with suppression of oncogenicity has proved a useful model to study inducer-mediated differentiation of transformed cells.

Recently, a class of compounds that induce differentiation, have been shown to inhibit histone deacetylases. Several experimental antitumor compounds, such as trichostatin A (TSA), trapoxin, suberoylanilide hydroxamic acid (SAHA), and phenylbutyrate have been shown to act, at least in part, by inhibiting histone deacetylases. Additionally, diallyl sulfide and related molecules, oxamflatin, MS-27-275, a synthetic benzamide derivative, butyrate derivatives, FR901228, depudecin, and m-carboxycinnamic acid bishydroxamide have been shown to inhibit histone deacetylases. In vitro, these compounds can inhibit the growth of fibroblast cells by causing cell cycle arrest in the G1 and G2 phases, and can lead to the terminal differentiation and loss of transforming potential of a variety of transformed cell lines. In vivo, phenylbutyrate is effective in the treatment of acute promyelocytic leukemia in conjunction with retinoic acid. SAHA is effective in preventing the formation of mammary tumors in rats, and lung tumors in mice.

U.S. Pat. No. 6,511,990 discloses amide derivatives of Formula:

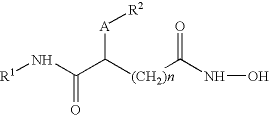

wherein A is an amido group and n is an integer between 3 and 8.

These compounds are histone deacetylase inhibitors particularly suitable for inducing growth arrest, terminal differentiation and/or apoptosis of neoplastic cells and thus inhibiting their proliferation.

HDAC inhibitors of Formula are disclosed in Kahnberg et al. (J. Med. Chem. 2006, 49, (26); 7611-7622):

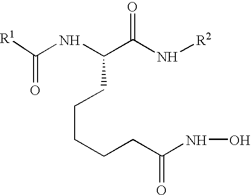

Hanessian et al. (Bioorg. Med. Chem. Lett. 2006, 16, 4784-4787) discloses molecules of Formulas:

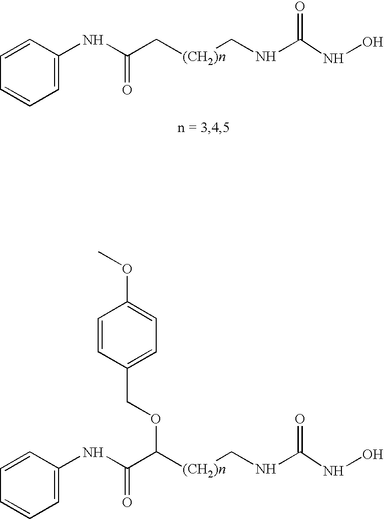

and their activity as histone deacetylase (HDAC) inhibitors. None of the disclosed molecules exhibited HDAC inhibitory activity below 1.0 microM. Furthermore, no cytotoxic activity on different tumor cell lines was seen below 20.0 microM.

There is the need to find new and/or alternatives compounds having an increased HDAC inhibitory activity.

### SUMMARY OF THE INVENTION

The aim of the present invention is to find novel compounds having anti-tumoral and pro-apoptotic activity.

The aforementioned objective has been met according to compounds of claim 1, to a composition of claim 12, to the use of claim 13. Preferred embodiments are set out within the dependent claims.

### DETAILED DESCRIPTION OF THE PREFERRED EMBODIMENTS

The following paragraphs provide definitions of the various chemical moieties that make up the compounds according to the invention and are intended to apply uniformly through-out the specification and claims unless an otherwise expressly set out definition provides a broader definition.

“C₁-C₃-alkyl” refers to monovalent alkyl groups having 1 to 3 carbon atoms.

“Alkylene” refers to a divalent alkyl chain.

“Aryl” refers to an unsaturated aromatic carbocyclic group of from 6 to 14 carbon atoms having a single ring (e.g. phenyl) or multiple condensed rings (e.g. naphthyl). Preferred aryl include phenyl, naphthyl, phenantrenyl and the like.

“Acyl” refers to the group C(O)R4 where R4 includes (C₁₋₄)alkyl.

“Pharmaceutically acceptable salts” refers to salts of the below identified compounds of Formula I that retain the desired biological activity. Examples of such salts include, but are not restricted to acid addition salts formed with inorganic acids (e.g. hydrochloric acid, hydrobromic acid, sulfuric acid, phosphoric acid, nitric acid, and the like), and salts formed with organic acids such as acetic acid, trifluoroacetic acid, oxalic acid, tartaric acid, succinic acid, malic acid, fumaric acid, maleic acid, ascorbic acid, benzoic acid, tannic acid, pamoic acid, alginic acid, polyglutamic acid, naphthalenesulfonic acid, naphthalene disulfonic acid, and polygalacturonic acid.

“Alkoxy” refers to O—R7 where R7 includes Alkyl, Alkenyl including allyl or (2-Me)Allyl, Alkynyl.

“Pharmaceutically active derivative” refers to any compound that upon administration to the recipient, is capable of providing directly or indirectly, the activity disclosed herein.

According to a first aspect of the invention, compounds of Formula I

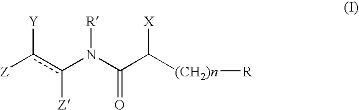

are provided.

In the compounds of Formula I:

the dotted line indicates an optional double bond;

R represents CONHOH, CONHCH₂SH, CONHCH₂SCOCH₃, SH, SCOCH₃, SCH₃, N(OH)COH, COCONHCH₃, CF₃;

n=1-7 and the alkylene chain is unsubstituted or substituted, preferably in a omega position, i.e. a position opposite to the R group of the alkylene chain, with one or more NH₂ groups, OH, (C₁₋₃)alkyl, SH, (C₁₋₇)alkoxy;

z and z′ are linked to form a phenyl group or a five- or six-membered heteroaromatic ring containing one to four nitrogen atoms, the phenyl group or the five- or six-membered heteroaromatic ring being unsubstituted or substituted with up to 4 substituents R″ or optionally condensed with an aryl or heteroaryl group;

X is selected from the group comprising OH, unsubstituted or substituted (C₁₋₇)-alkoxy group, O—CH₂-Aryl, where aryl is unsubstituted or substituted with one or two substituents, which are the same or different and are selected from the group comprising H, NH₂, NH—(C₁₋₃)Alkyl, CN, NO₂, (C₁₋₃)Alkyl unsubstituted or substituted with halogen, O—(C₁₋₃)Alkyl, Halogen, aryl, O-Aryl;

Y is selected from the group comprising H, OH, O—(C₁₋₃)Alkyl, NH₂, NH—(C₁₋₃)Alkyl, Halogen;

Or X and Y form a cycle wherein X and Y are linked by a bridge of Formula A selected from the group consisting of:

X—(C₁₋₄)Alkylene(R1)-W—(C₁₋₄)Alkylene-Y X—(C₂₋₄)Alkenylene(R1)-W—(C₁₋₄)Alkylene-Y

wherein X and Y are the same or different and are selected from the group consisting of —O—, —NH— unprotonated or protonated, —S—, —CH₂—, (C₁₋₃)-Alkylene-O—;

W is either absent or it represents an arylene group selected from the group comprising:

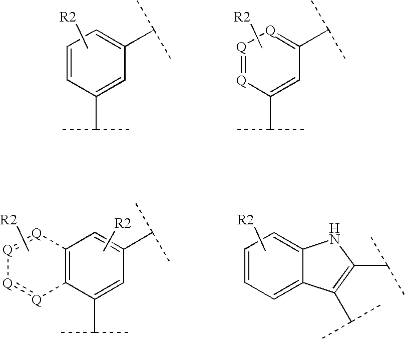

R′ represents H, (C₁₋₅)Alkyl, CH₂-Aryl unsubstituted or substituted with H, O—(C₁₋₃)Alkyl, OH and nitro;

R″ represents H, NH₂, NH—(C₁₋₃)Alkyl, NHCO(C₁₋₃)Alkyl, O—(C₁₋₃)Alkyl, (C₁₋₃)Alkylene-NH₂, (C₁₋₃)Alkylene-NHCO(C₁₋₃)Alkyl, (C₁₋₃)Alkyl, NH-acyl, (C₁₋₃)Alkylene-NH-acyl, OH;

R1 represents H, halogen, NO₂, (C₁₋₃)Alkyl-NH₂, OH, NH₂ unsubstituted or substituted with a (C₁₋₃)acyl group, phenyl group unsubstituted or substituted with a —O—(C₁₋₃)Alkyl;

R2 represents H, (C₁₋₅)Alkyl, ±O—(C₁₋₃)Alkyl, halogen, NO₂, NH₂ unsubstituted or substituted with a (C₁₋₃)acyl group or a (C₁₋₃)Alkyl, OH, CN, COOR3 where R3 is selected from the group consisting of H, (C₁₋₃)Alkyl; and

Q represents CH, N or, for saturated derivatives, CH₂, NH.

The present invention also includes geometrical isomers, in an optically active form as enantiomers, diastereomers, as well as in the form of racemate, as well as pharmaceutically acceptable salts of the compound of Formula I.

Preferably, X and Y form a cycle to obtain compounds of Formula II:

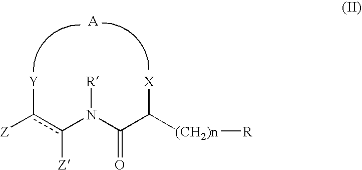

wherein z, z′, Y, A, X, n, R′ and R are as defined above.

Most preferred bridge of Formula A is selected from the group consisting of —(CH₂)₃—, —(CH₂)₄—, —(CH₂)₅—, and

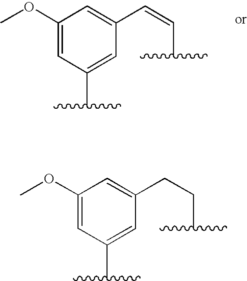

Most preferred substituent R is CONHOH and preferably n ranges from 4 to 6.

Preferably z and z′ are linked to form a phenyl group or a five- or six-membered heteroaromatic ring selected from the group comprising pyridine, pyrazole and pyrrole.

Preferably, substituent R″ is selected from the group consisting of H, —CH₃, —OCH₃, —NHCOCH₃, —NH₂, —CH₂NH₂, —CH₂NHCOCH₃.

Specific examples of compounds of Formula I include the following:

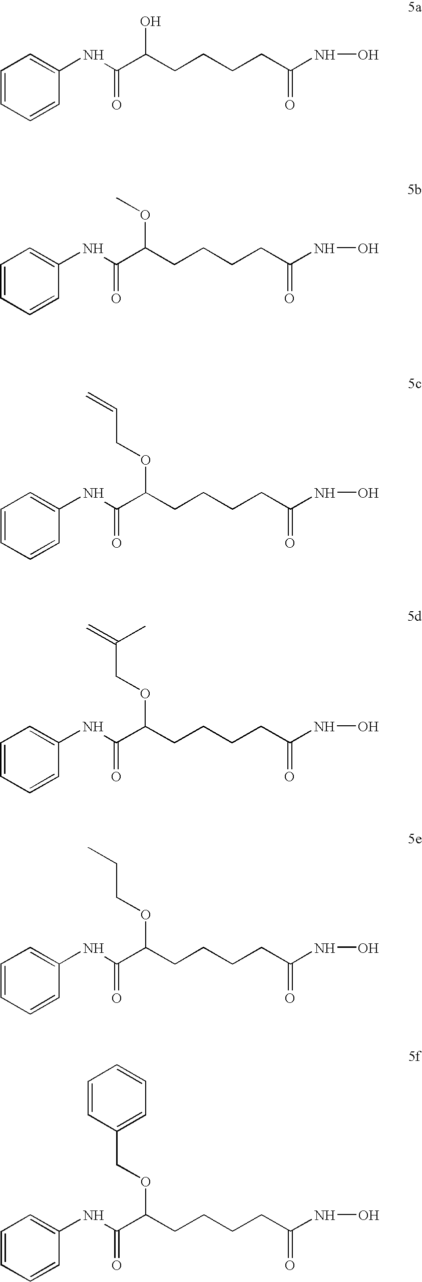

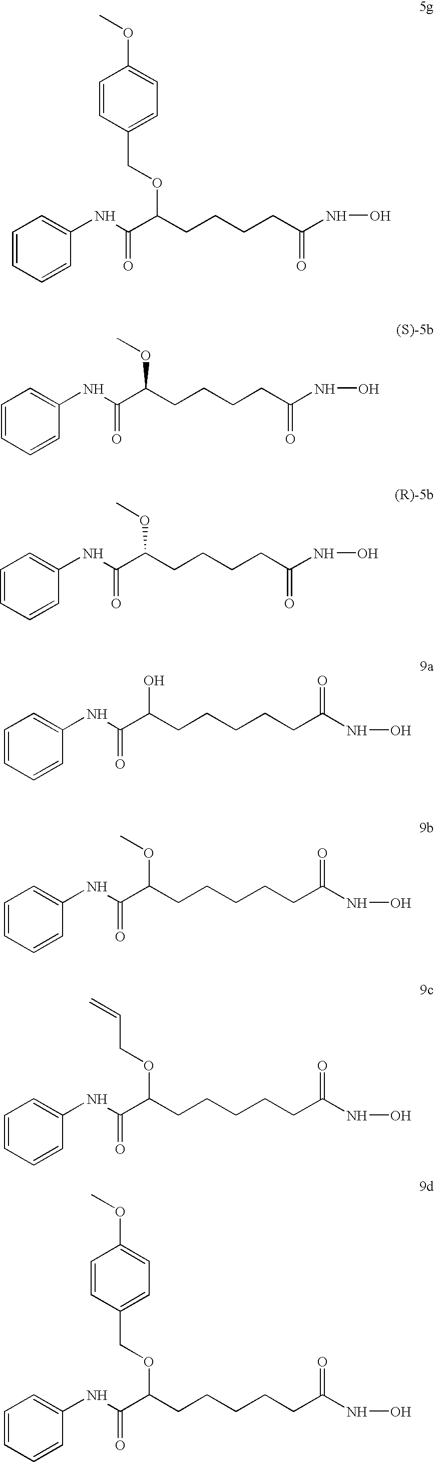

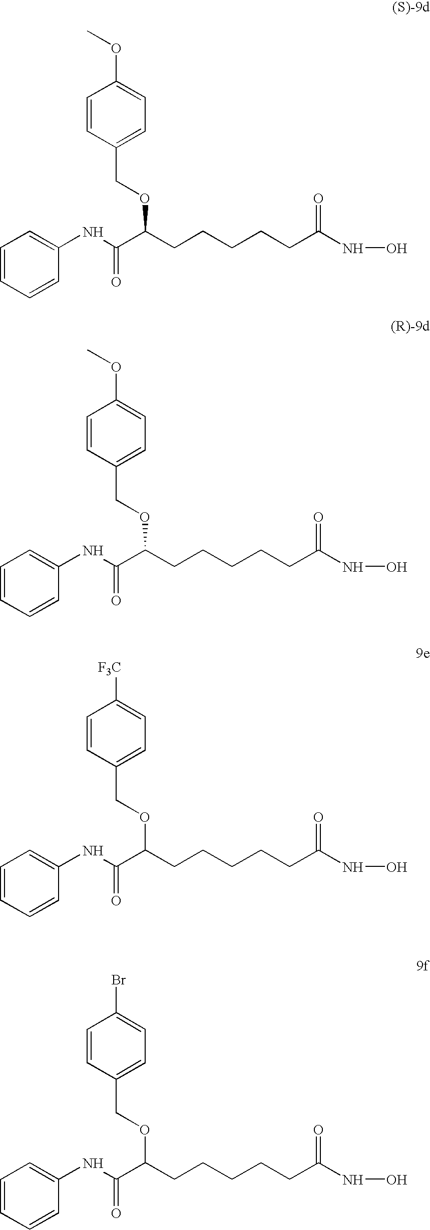

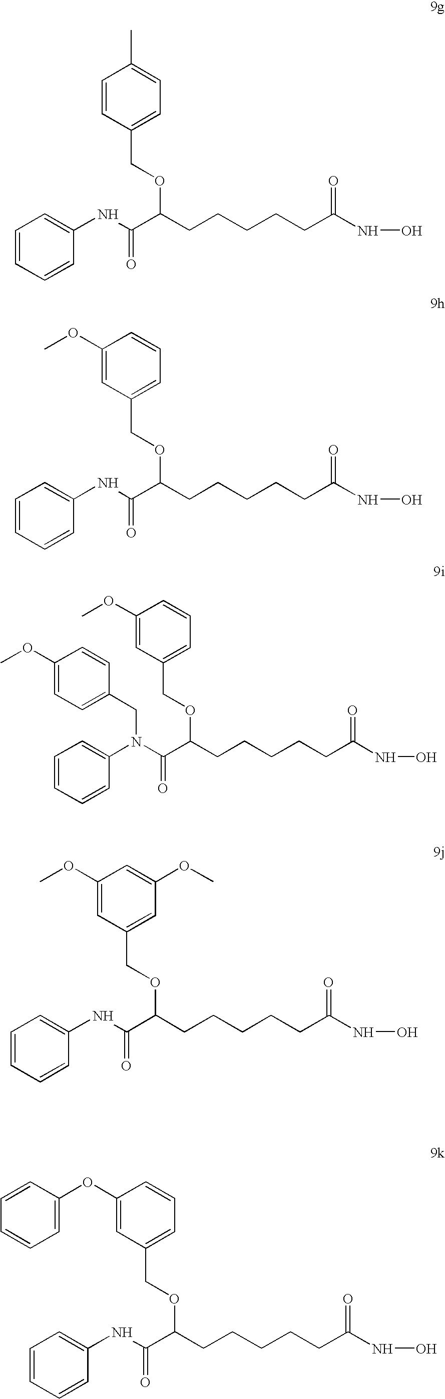

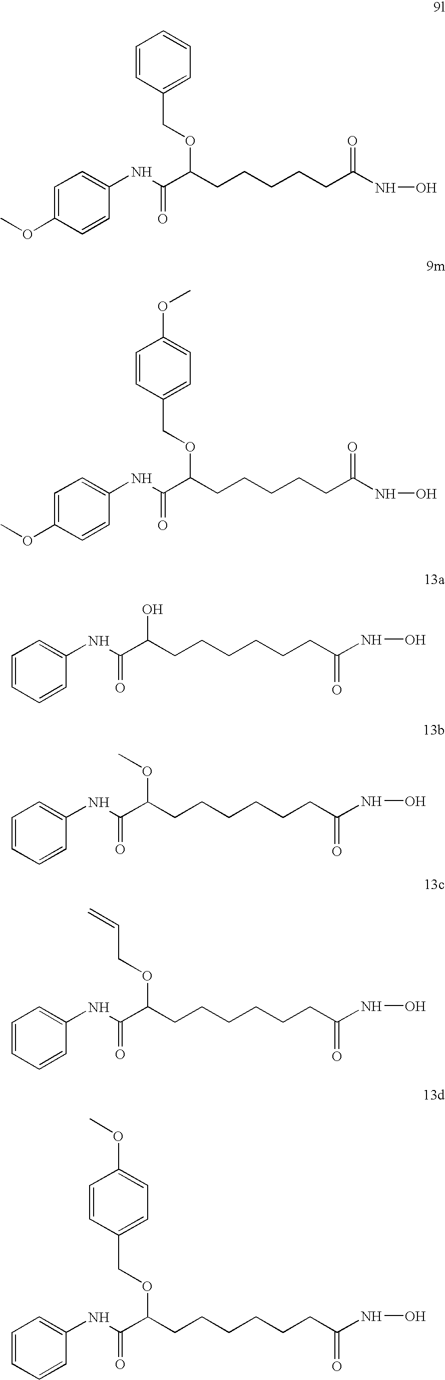

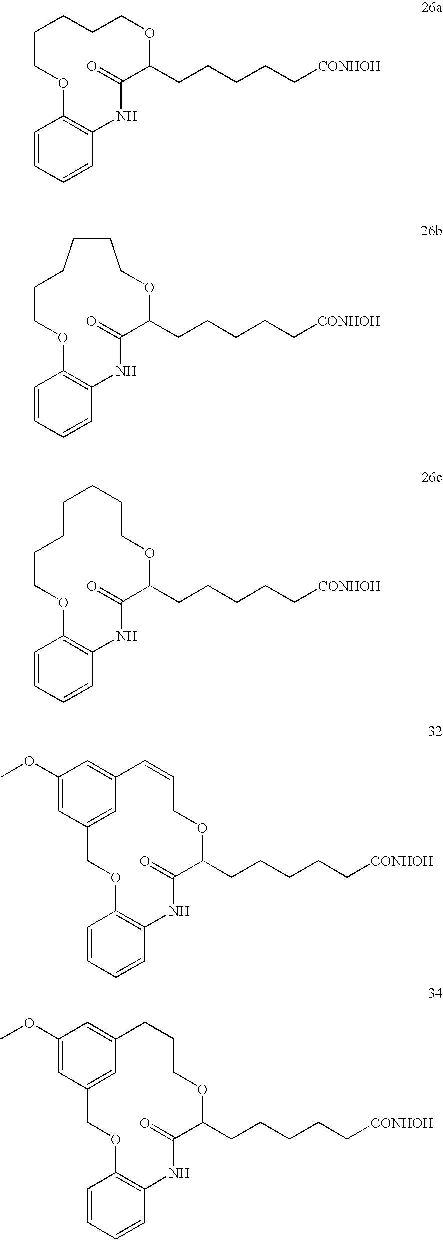

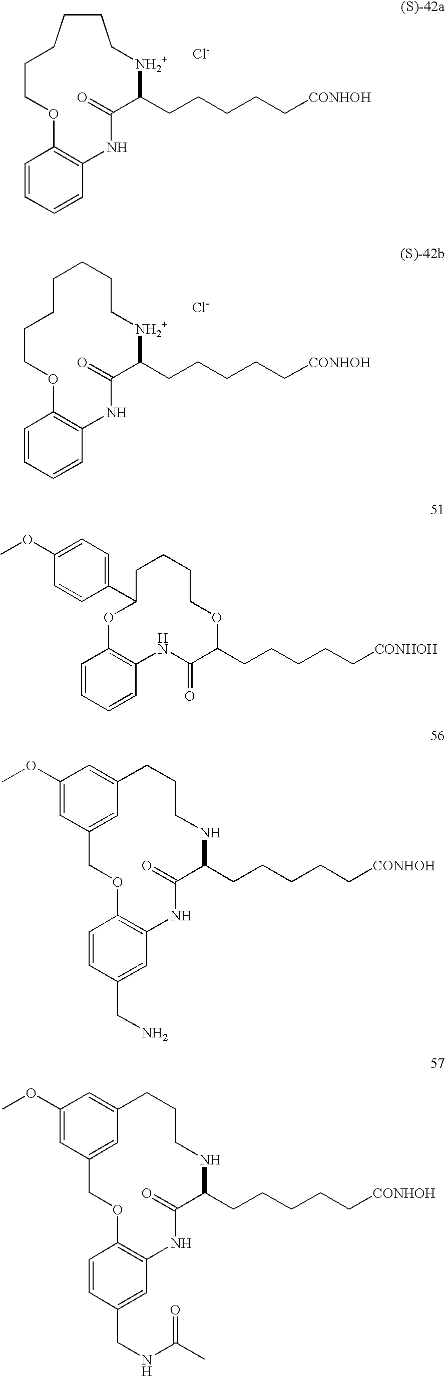

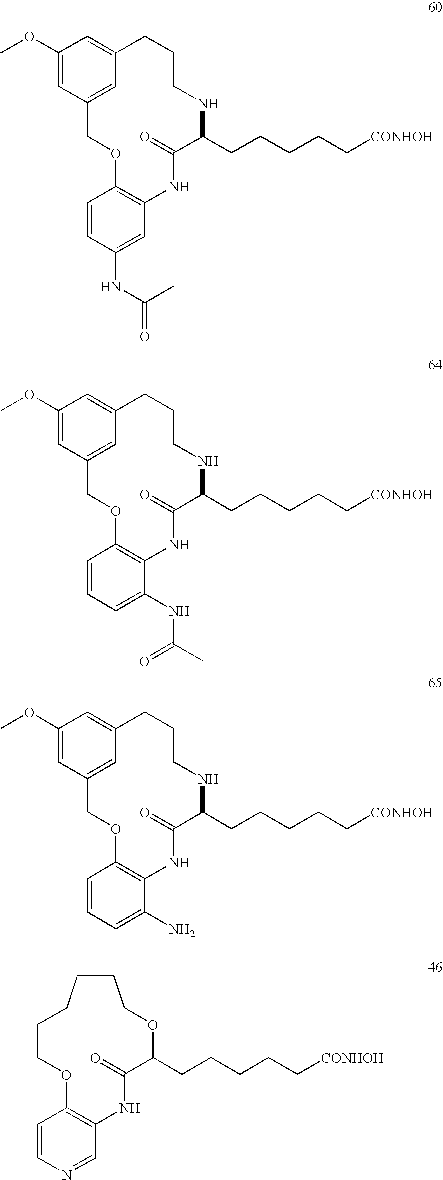

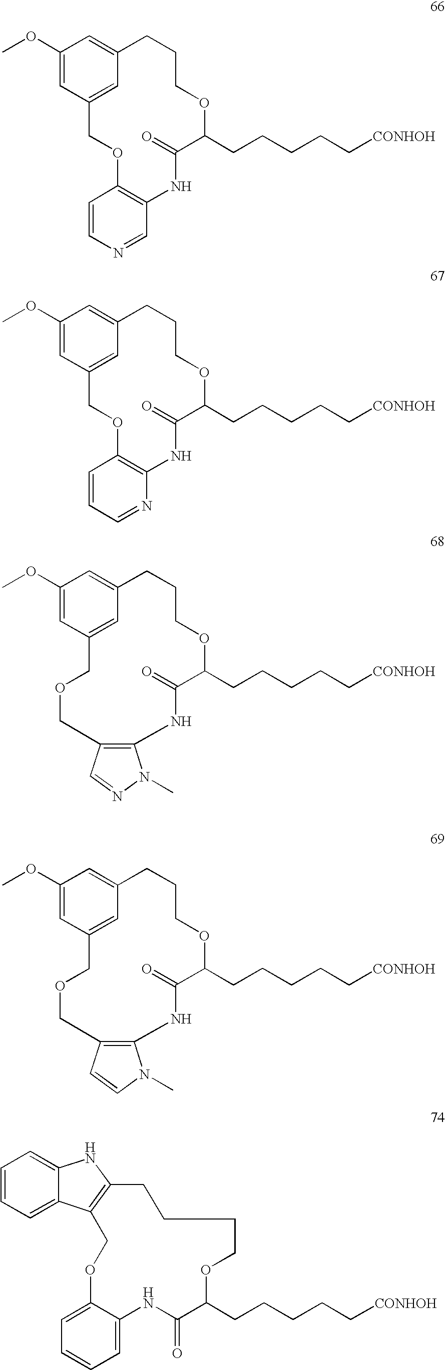

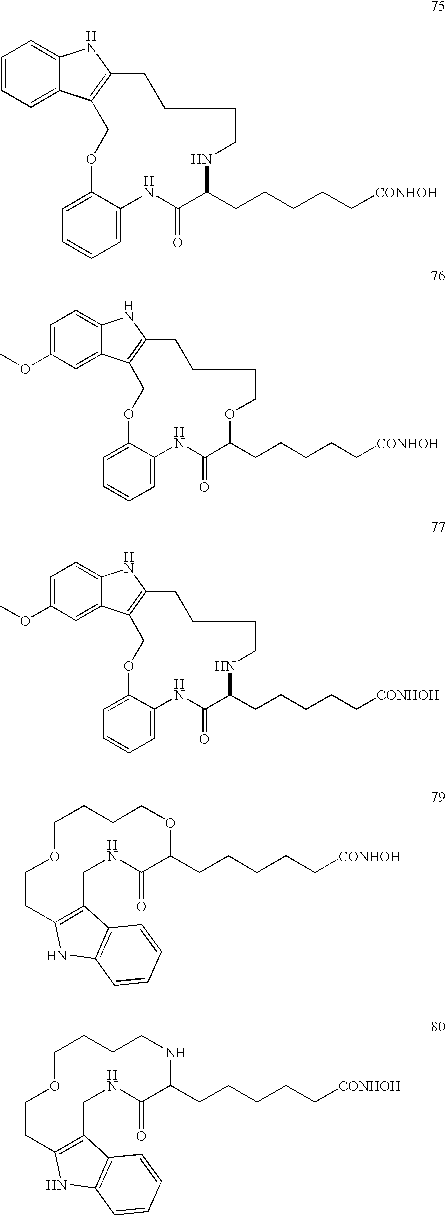

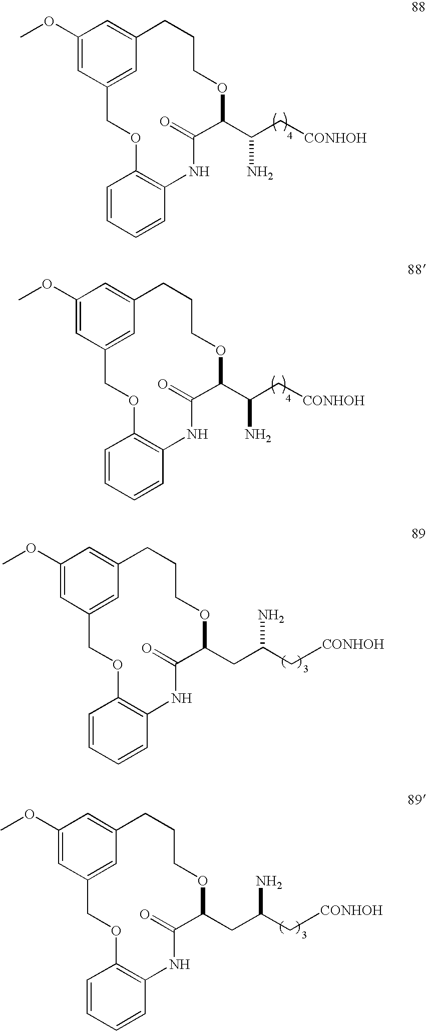

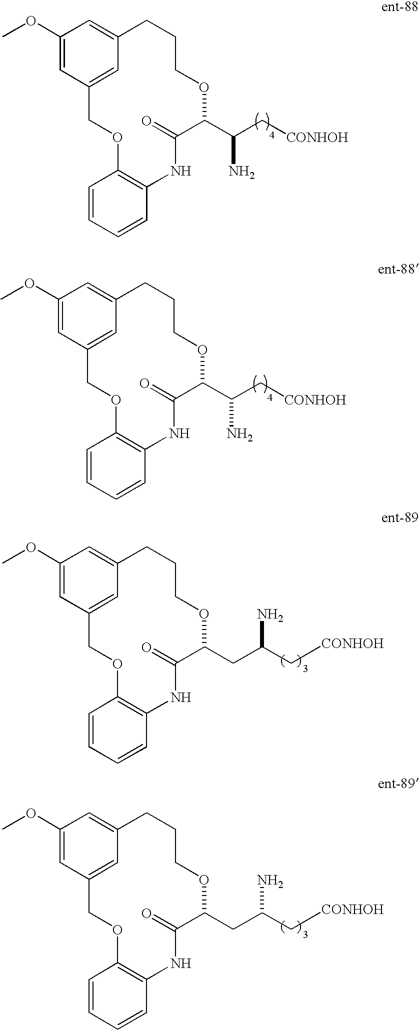

The most preferred compounds are those which are selected from the group consisting of 9a, 9b, 9d, (S)-9d, (R)-9d, 9e, 9f, 9g, 9h, 9j, 9k, 9l, 9m, 13d, 26b, 26c, 32, 34.

A further aspect of the present invention is related to a pharmaceutical composition comprising a compound of Formula I according to the invention and a pharmaceutically acceptable carrier, stabilizer, diluent or excipient thereof.

For any compound, the therapeutically effective dose can be estimated initially either in cell culture assays or in animal models, usually mice, rats, guinea pigs, rabbits, dogs or pigs.

The animal model may also be used to determine the appropriate concentration range and route of administration. Such information can then be used to determine useful doses and routes for administration in humans.

The precise effective dose for a human subject will depend upon the severity of the disease state, general health of the subject, age, weight, and gender of the subject, diet, time and frequency of administration, drug combination (s), reaction sensitivities, and tolerance/response to therapy. This amount can be determined by routine experimentation and is within the judgement of the clinician. Generally, an effective dose will be from 0.01 mg/kg to 100 mg/kg, preferably 0.05 mg/kg to 50 mg/kg. Compositions may be administered individually to a patient or may be administered in combination with other agents, drugs or hormones.

Dosage treatment may be a single dose schedule or a multiple dose schedule.

A further embodiment of the invention is a process for the preparation of pharmaceutical compositions characterised by mixing one or more compounds of Formula I with suitable excipients, stabilizers and/or pharmaceutically acceptable diluents.

When employed as pharmaceuticals, the compounds of the present invention are typically administered in the form of a pharmaceutical composition. Hence, pharmaceutical compositions comprising a compound of the invention and a pharmaceutically acceptable carrier, diluent or excipient therefore are also within the scope of the present invention. A person skilled in the art is aware of a whole variety of such carrier, diluent or excipient compounds suitable to formulate a pharmaceutical composition.

The compounds of the invention, together with a conventionally employed adjuvant, carrier, diluent or excipient may be placed into the form of pharmaceutical compositions and unit dosages thereof, and in such form may be employed as solids, such as tablets or filled capsules, or liquids such as solutions, suspensions, emulsions, elixirs, or capsules filled with the same, all for oral use, or in the form of sterile injectable solutions for parenteral (including subcutaneous use). Such pharmaceutical compositions and unit dosage forms thereof may comprise ingredients in conventional proportions, with or without additional active compounds or principles, and such unit dosage forms may contain any suitable effective amount of the active ingredient commensurate with the intended daily dosage range to be employed.

Pharmaceutical compositions containing a compound of this invention can be prepared in a manner well known in the pharmaceutical art and comprise at least one active compound. Generally, the compounds of this invention are administered in a pharmaceutically effective amount. The amount of the compound actually administered will typically be determined by a physician, in the light of the relevant circumstances, including the condition to be treated, the chosen route of administration, the actual compound administered, the age, weight, and response of the individual patient, the severity of the patient's symptoms, and the like.

The pharmaceutical compositions of the present invention can be administered by a variety of routes including oral, rectal, transdermal, subcutaneous, intravenous, intramuscular and intranasal. The compositions for oral administration can take the form of bulk liquid solutions or suspensions, or bulk powders. More commonly, however, the compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include pre-filled, pre-measured ampoules or syringes of the liquid compositions or pills, tablets, capsules or the like in the case of solid compositions.

Liquid forms suitable for oral administration may include a suitable aqueous or non-aqueous vehicle with buffers, suspending and dispensing agents, colorants, flavours and the like. Solid forms may include, for example, any of the following ingredients, or compounds of a similar nature: a binder such as microcrystalline cellulose, gum tragacanth or gelatine; an excipient such as starch or lactose, a disintegrating agent such as alginic acid, Primogel or corn starch; a lubricant such as magnesium stearate; a glidant such as colloidal silicon dioxide; a sweetening agent such as sucrose or saccharin; or a flavouring agent such as peppermint, methyl salicylate, or orange flavouring.

Injectable compositions are typically based upon injectable sterile saline or phosphate-buffered saline or other injectable carriers known in the art.

The above described components for orally administered or injectable compositions are merely representative.

The compounds of this invention can also be administered in sustained release forms or from sustained release drug delivery systems.

A further aspect of the present invention is related to the use of a compound of Formula I or of the pharmaceutical composition thereof according to the present invention for the preparation of a medicament.

In a preferred embodiment the medicament is suitable for selectively inducing terminal differentiation of neoplastic cells and thereby inhibiting proliferation of such cells, inducing differentiation of tumor cells in a tumor or inhibiting the activity of histone deacetylase.

In a most preferred embodiment the medicament is suitable for the treatment of primary cancers as well as secondary cancers.

More preferably, the medicament is useful in the treatment of leukaemia, colon cancer and lung cancer.

The compounds exemplified in this invention may be prepared from readily available starting materials using the following general methods and procedures. It will be appreciated that where typical or preferred experimental conditions (i.e. reaction temperatures, time, moles of reagents, solvents etc.) are given, other experimental conditions can also be used unless otherwise stated. Optimum reaction conditions may vary with the particular reactants or solvents used, but such conditions can be determined by the person skilled in the art, using routine optimisation procedures.

Generally, the compounds according to the general formula I may be obtained by several processes using solution-phase chemistry protocols.

Macrocyclic hydroxamic acids can be assembled according to the synthetic analysis depicted in Chart 1, through the application of general procedures (1-8).

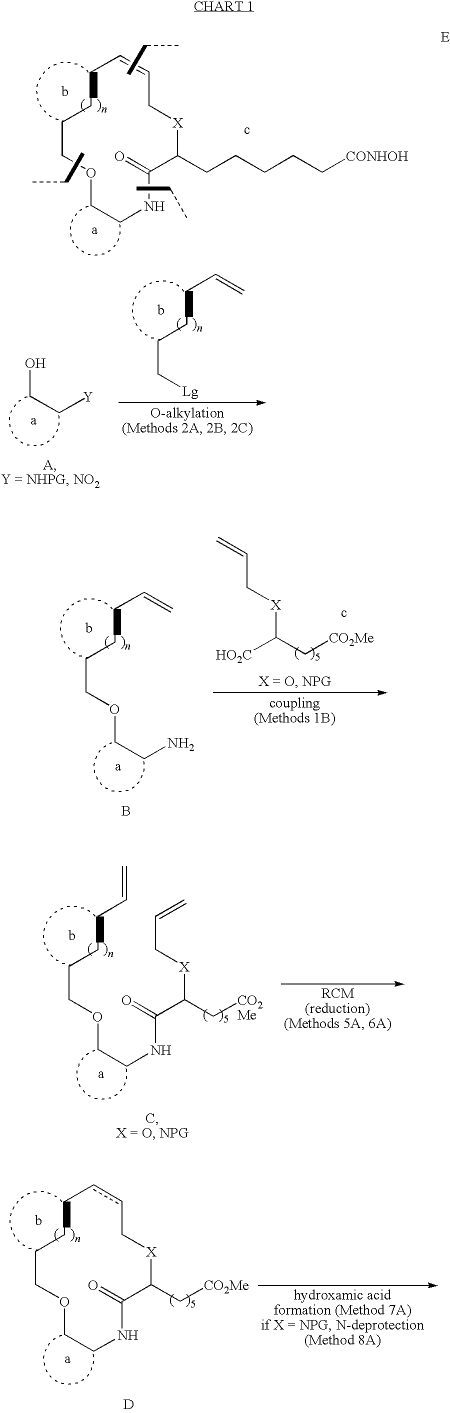

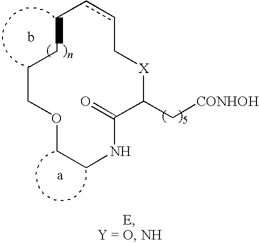

### General Procedures

1. Amide Bond Formation

Method 1A. Coupling with free 2-hydroxy acids (Chidambaram, R; Zhu, J.; Penmetsa, K.; Kronenthal, D.; Kant, J. Tetrahedron Lett. 2000 41, 6017-6020)

In a typical procedure, the 2-hydroxy alkene dioic acid ω-methyl ester (1.0 eq) and 1,2,4-triazole (1.4 eq) were stirred in anhydrous CH₂Cl₂ (1.4 mL/mmol hydroxy acid) under an atmosphere of argon until a clear solution was obtained. The solution was then cooled to 0° C. and the suitable N-sulfinylaniline derivative (Kim, Y. H.; Shin, J. M. Tetrahedron Lett. 1985 26, 3821-3824) (1.4 eq) dissolved in CH₂Cl₂ (0.4 mL/mmol) was added. After stirring for 2 h at 0° C., the reaction mixture was allowed to react for 48 h before quenching by addition of NH₄Cl (aq, sat.). The organic phase was separated and the extraction was continued with further portions of CH₂Cl₂. All the combined organic extracts were dried (MgSO₄), filtered, and concentrated under reduced pressure. Flash chromatographic purification (hexanes/EtOAc) afforded pure anilides (see examples 4a, 8a′, 8a″, 12a) in 84-96% yield.

Method 1B. Coupling with 2-alkoxy carboxylic acids

Method 1B1. In a typical procedure the 2-alkoxy carboxylic acid intermediate and the suitable aromatic amine (1.5-1.7 eq) were dissolved in anhydrous CH₂Cl₂ (5 mL/mmol carboxylic acid) under an argon atmosphere, then N-ethyl-N′-(3-dimethylaminopropyl)carbodiimide (EDC, 3.5 eq), 1-hydroxybenzotriazole (HOBt, 1.3 eq) and diisopropylethylamine (DIEA, 3.5 eq) were sequentially added at 0° C. The mixture was slowly warmed to room temperature and stirring was continued for 24 h. The reaction was quenched with NH₄Cl (aq, sat.) and extracted with CH₂Cl₂. All the combined organic layers were dried (MgSO₄), concentrated under vacuum and purified by flash chromatography (hexanes/EtOAc), to afford pure amides (see examples (S)-4b, (S)-8a′, 23a-c, 30, 49) in 60-79% yield.

Method 1B2. (Li, H.; Jiang, X.; Ye, Y.-H.; Fan, C.; Romoff, T.; Goodman, M. Org. Lett. 1999, 1, 91-93) In a typical procedure, a 0.1 mM solution of the 2-alkoxy carboxylic acid intermediate in anhydrous THF was stirred for 30 min in the presence of 3-(diethoxyphosphoryloxy)-1,2,3-benzotriazin-4(3H)-one (DEBPT) (2.0 equiv) and DIPEA (2.0 equiv) under argon atmosphere, and to the resulting bright yellow solution the suitable aromatic amine (1.5-2.0 equiv) was added. After stirring for 24-36 h, the reaction was quenched with NH₄Cl (aq., sat.), and extracted with EtOAc. The organic phase was washed with NaHCO₃ (aq., sat.), and brine, dried (MgSO₄), and concentrated in vacuo to afford a crude which was purified by flash chromatography (hexanes/EtOAc). (see examples (S)-40a-b, 44) in 45-65% yield.

2. O-Alkylation Reactions

Method 2A. Synthesis of ω-alkoxy-ω-phenylcarbamoyl alkanoic acid methyl esters. Free alcohol intermediates (see examples 4a, 8a′, 8a″, 12a) (1.0 eq) and the suitable bromide or iodide (5-10 eq) were dissolved in anhydrous MeCN (see examples 4b, 4c, 4d, 8b, 8c, 12b, 12c), or DMF (see examples 4f, 8f, 8g, 8h, 8l, 8j, 8k, 8l) or toluene (see examples 8g, 8e, 12d) (1.5 mL/mmol) under an argon atmosphere, to which Ag₂O (1.2-2 eq) was added. The heterogeneous mixture was allowed to react overnight under stirring at room temperature or at 45° C. After filtration of the solids through a pad of Celite® and removal of the solvent under reduced pressure, the crude residue was purified by flash chromatography (hexanes/EtOAc), which afforded the pure ω-alkoxy alkanoic acid methyl ester intermediate in 24-80% for one cycle reaction.

Method 2B. Synthesis of ω-p-methoxybenzyloxy-ω-phenylcarbamoyl alkanoic acid methyl esters. In a typical procedure, to a solution of the suitable alcohol in anhydrous Et₂O (1.5 mL/mmol) under an argon atmosphere a freshly prepared solution of p-methoxybenzyl-trichloroacetimidate (0.5 M, 2.0 eq) (Audia, J. E., Boisvert, L.; Patten, A. D.; Villalobos, A.; Danishefsky, S. J. J. Org. Chem. 1989, 54, 3738-3740) was added. After cooling at 0° C. catalytic BF₃.Et₂O (0.01 eq) was added, and the solution was allowed to reach room temperature during 2 h under stirring while developing a white precipitate. The mixture was filtered through Celite®, and the solid was washed with n-hexane. The filtrate was washed with a saturated aqueous solution of NaHCO₃, dried over MgSO₄, and concentrated in vacuo. After purification by flash chromatography (gradient 9:1 to 7:3 hexanes/EtOAc), pure p-methoxybenzyl ether intermediates (see examples 8d, 8m, (S)-8d, and (R)-8d) were recovered in 35-39% yield.

Method 2C. Synthesis of O-alk-1-enyloxy-nitroaryls and O-alk-1-enyloxy-nitroheteroaryls.

Method 2C1. In accord to a published procedure (Beckwith, A. L. J.; Gara, W. B.; J. Chem. Soc., Perk. Trans. 11975, 593-600 and Ventrice, T.; Campi, E. M.; Jackson, W. R., Patti, A. F. Tetrahedron 2001 57, 7557-7574), a stirred mixture of nitrohydroxy(hetero)aryl (1.0 eq), ω-bromo-1-alkene (1.1 eq), and Na₂CO₃ (1.1 eq) in H₂O (0.7 mL/mmol Na₂CO₃) was refluxed for 48 h. After dilution with H₂O the mixture was extracted with CH₂Cl₂. The organic phase was dried (MgSO₄), concentrated in vacuo, and purified by flash chromatography (hexanes/EtOAc) to afford the O-alk-1-enyloxy nitro(hetero)aryl derivative. (See examples 22a-c)

Method 2C2. In a typical procedure, to a solution of diisopropyl azodicarboxylate (DIAD, 1.0-1.5 equiv) and nitrohydroxy(hetero)aryl (1.0-1.5 equiv) in anhydrous THF or toluene [10 mL/mmol nitrohydroxy(hetero)aryl], alcohol derivative (1.0 equiv) was added dropwise while stirring at 0° C. under argon, followed by Ph₃P (1.0-1.5 equiv). The reaction mixture was then warmed to room temperature and allowed to react until complete conversion of the starting material. After 2 days the solvent was removed under reduced pressure, and the residue purified by flash chromatography (hexanes/EtOAc) to afford the pure O-alk-1-enylated nitro(hetero)aryl derivative. (see examples 22a-c, 28, 43, 48).

3. Reduction of Nitro(Hetero)Aryl Intermediates to Amino Derivatives

Method 3A1. Reduction of nitroaryl derivatives was performed using a modified published procedure (Fletcher R. J.; Lampard, C.; Murphy, J. A.; Lewis, N. J. Chem. Soc., Perk. Trans. 11995, 623-629). To a suspension of Cu(acac)₂ (0.2 eq) in EtOH (90 mL/mmol Cu(acac)₂) a solution of NaBH₄ (1.0 eq) in EtOH (1.8 mL/mmol NaBH₄) was added under an argon atmosphere. Once the hydrogen evolution had decreased, a solution of the nitrophenol derivative (1.0 eq) in EtOH (10.0 mL) was added, followed by a further portion of NaBH₄ (2.0 eq) in EtOH (0.9 mL/mmol). The reaction mixture was vigorously stirred at room temperature. During this period further portion of NaBH₄ (7×1.0 eq) in EtOH (0.9 mL/mmol) were constantly added until complete conversion of the starting material. After 24 h the reaction was quenched with H₂O, and extracted with CH₂Cl₂. After concentration in vacuo the organic extract was taken up in 4N HCl, and washed with CH₂Cl₂. The acidic phase was then neutralized with NaOH, and extracted with CH₂Cl₂. Removal of the organic solvent left pure aniline derivative (see compound 22c) in 61-66% yield. In a different work-up the reaction was quenched with H₂O, and extracted with CH₂Cl₂. The organic phase was dried (MgSO₄), the solvent removed under vacuum, and the crude purified by flash chromatography (CH₂Cl₂) to give the pure aniline intermediate (see example 29).

Method 3A2. [(a) Bartra, M.; Romea, P.; Urpi, F.; Vilarrasa, J. Tetrahedron 1990, 46, 587-594. (b) Lebreton, J.; Waldner, A.; Leseuer, C.; De Mesmaeker, A. Synlett 1994, 137-140.] In a typical procedure, the nitro(hetero)aryl intermediate was dissolved in EtOAc (0.2 mM) and stirred in the presence of SnCl₂.2H₂O (5.0 equiv) until complete conversion of the starting material (48-72 h). The reaction mixture was neutralized with NaOH, and extracted with EtOAc. The organic phase was washed with brine, and concentrated in vacuo to afford in quantitative yield the pure amine, which was immediately dissolved in anhydrous in THF or CH₂Cl₂, and stored at 0° C. (See examples 22a-c, 29, 43, 48).

4. Carbon Chain Elongation Via Cross Metathesis

Method 4A. In a typical procedure, to a solution of the suitable protected terminal olefin intermediate in CH₂Cl₂ (2 mL/mmol) and methyl acrylate or methyl-3-butenoate (12 eq), Grubbs' catalyst 2^{nd} generation (0.03 eq) was added in one portion under argon, and the solution was stirred overnight at room temperature. The solvent was removed in vacuo, and the crude residue purified by flash chromatography (hexanes/EtOAc) which gave the unsaturated ester intermediate in 93-95% yield (see examples 15, (R/S)-18, (R)-18, (S)-18).

5. Macrocycle Formation Via Ring Closing Metathesis

Method 5A. In a typical procedure, to a 1 mM solution of the terminal diene intermediate in anhydrous CH₂Cl₂ under an argon atmosphere, Grubbs' catalyst 2^{nd} generation (0.1-0.3 equiv) was added portionwise. After stirring at ambient temperature for 24 h, or at 40° C. for 1-2 h, the solvent was removed under vacuum, and the residue purified by flash chromatography (hexanes/EtOAc). Pure unsaturated macrocycles were obtained in yield ranging from 45% to 99% (see examples 24a-c, 31, (S)-41a-b, 45, 50).

6. Double Bond Hydrogenation Following Metathesis Reactions

Method 6A. Olefin intermediates from cross and ring-closing metathesis reaction were reduced under conventional conditions. Unless stated otherwise, olefins were dissolved in MeOH (1.0 mL/0.1 mmol) and catalytic 10% palladium on carbon was added. The reaction vessel was evacuated by aspiration and thoroughly purged with H₂ (three times) and the resulting heterogeneous mixture was stirred under a balloon of H₂. After 4-24 h the H₂ was evacuated, the catalyst filtered off, and the filtrate concentrated under reduced pressure to give a crude which was subjected to flash chromatography (hexanes/EtOAc). Saturated intermediates were usually obtained in 85-99% yield.

7. Hydroxamic Acid Formation

Method 7A. In a typical procedure, to a solution of methyl ester in MeOH at 0° C., HONH₂ (50% aq solution, 15 eq) was added, followed by 1.0 N NaOH (10 eq). The mixture was stirred at 0° C. for 2-4 h, warmed slowly to rt, and stirred overnight. After careful neutralization with 1.0 N HCl the resulting mixture was extracted with EtOAc. The organic phase, dried (MgSO₄) and concentrated under vacuum, furnished a crude which was purified by flash chromatography (EtOAc or 9:1 EtOAc/MeOH), which afforded the pure hydroxamic acids in yields ranging from 65 and 99%. In particular cases, extremely polar hydroxamic acids were isolated by concentration of the aqueous mother phase, and purified from the residual salts by filtration of a methanolic solution of the crude product. (see example 45).

8. N-Boc Deprotection

Method 8A. Unless stated otherwise, N-Boc protected intermediates were dissolved in anhydrous 4.0 M HCl-dioxane or 3.0 N HCl-MeOH under argon atmosphere and stirred at rt for 30 min. n-Hexane was added, and the mixture was concentrated at reduced pressure. The crude was washed with anhydrous Et₂O, leaving pure hydrochloride salt as a crystalline solid in 90-99% yield.

A few examples of synthetic schemes are reported below in Schemes 1 to 15.

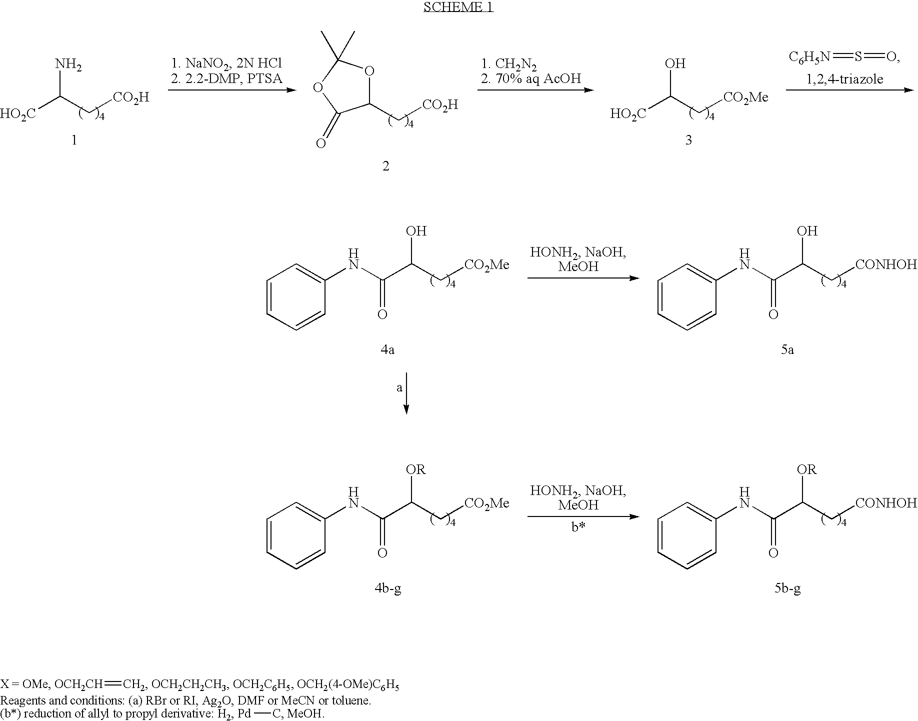

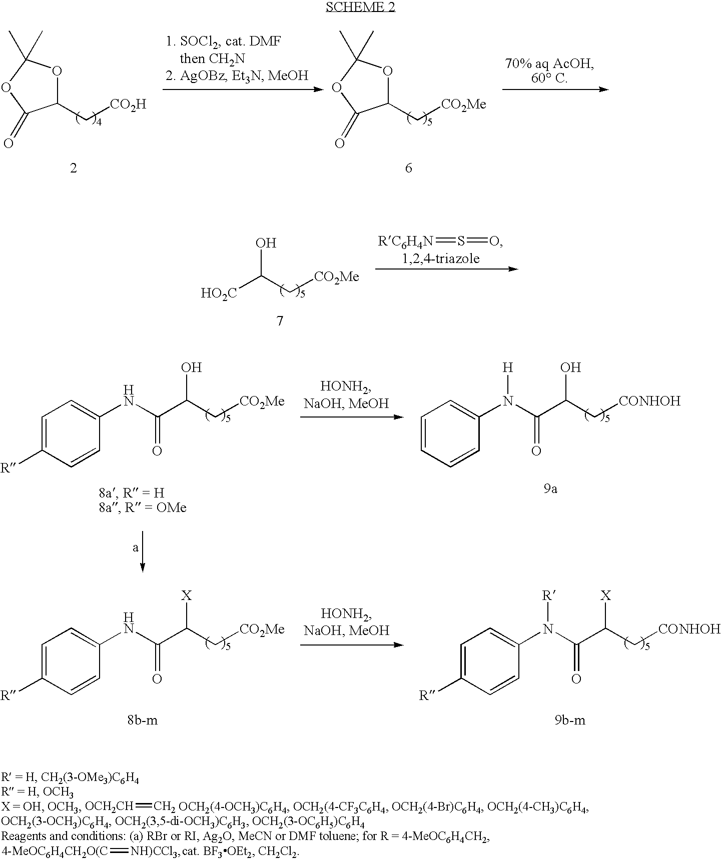

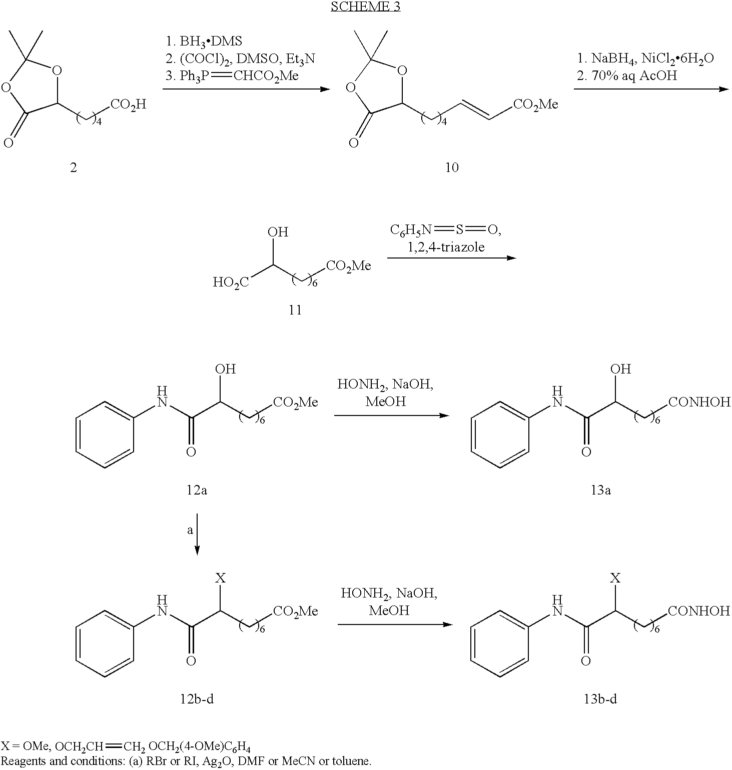

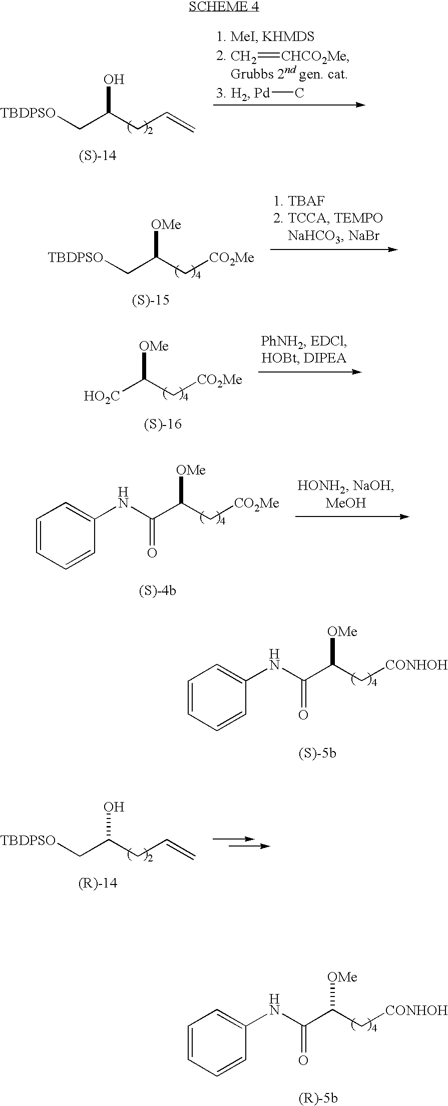

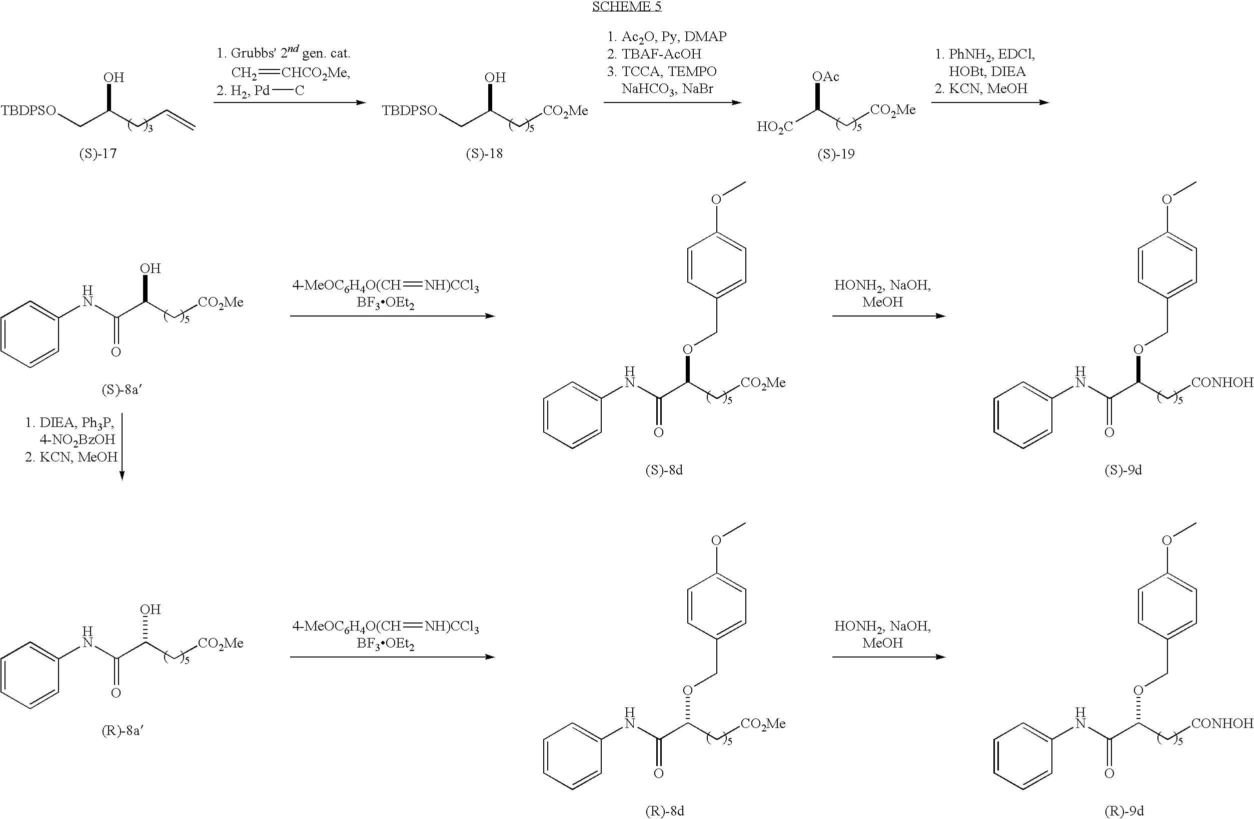

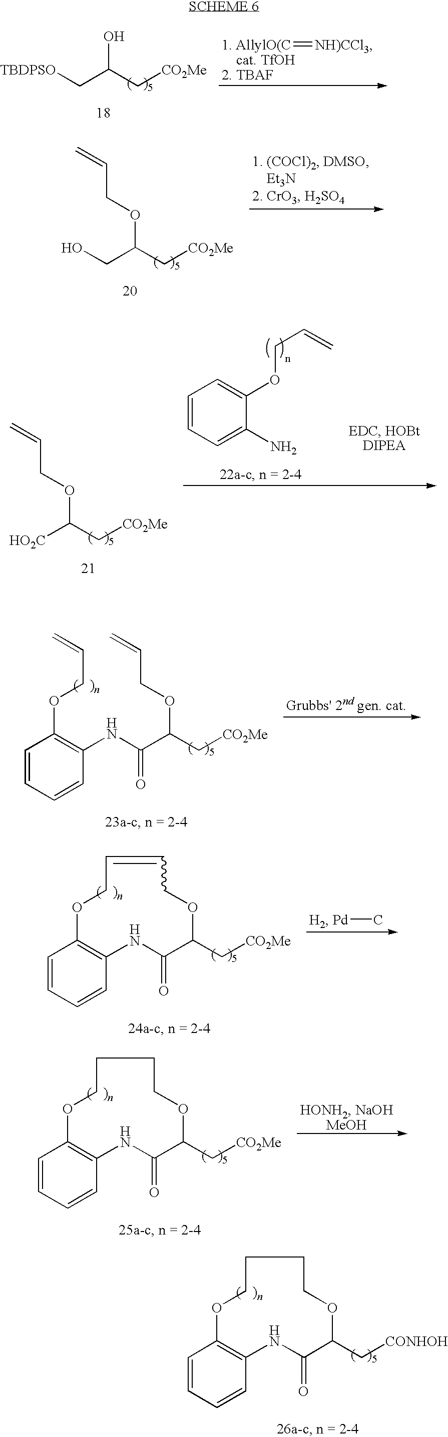

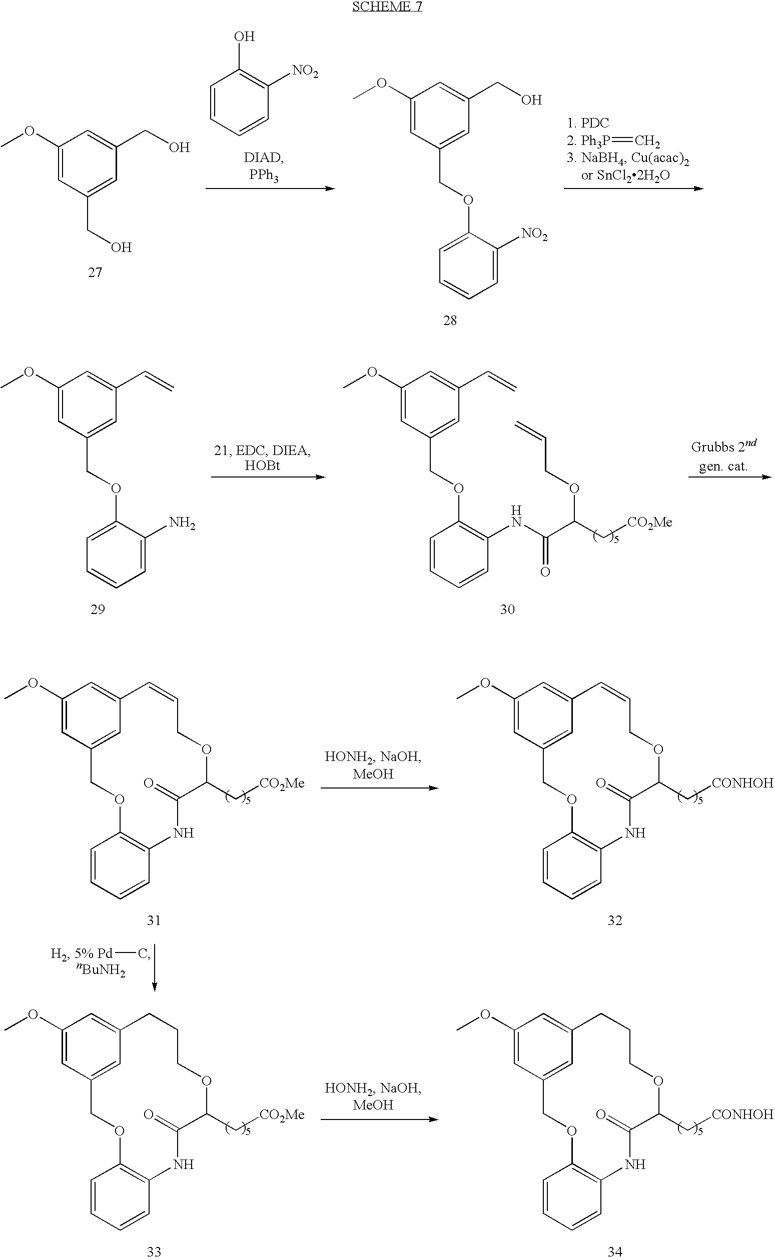

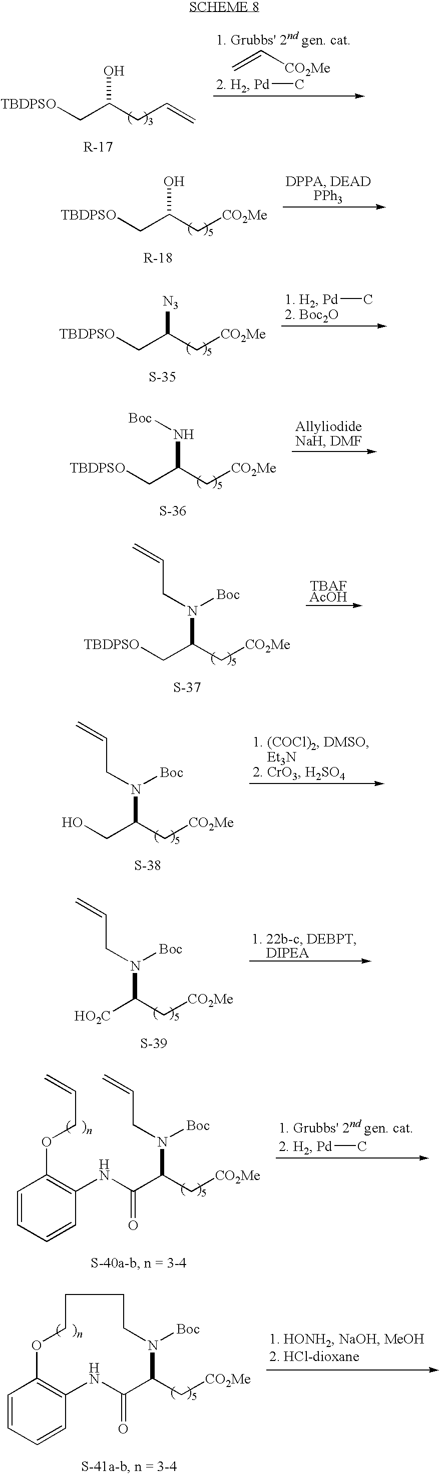

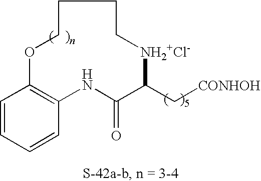

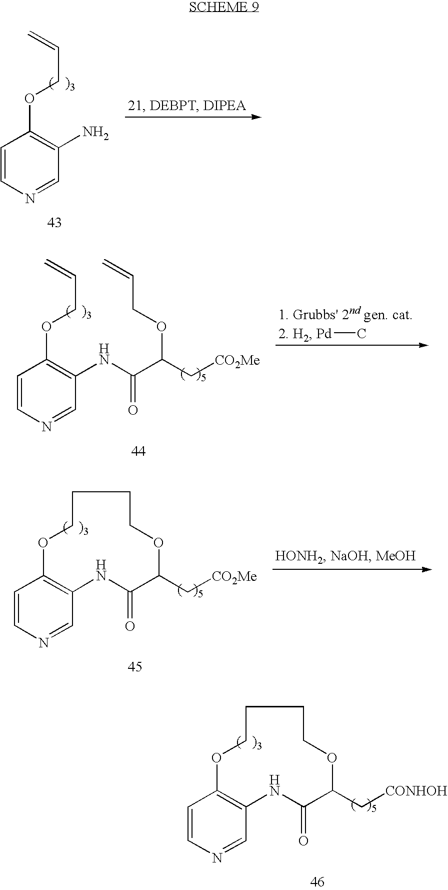

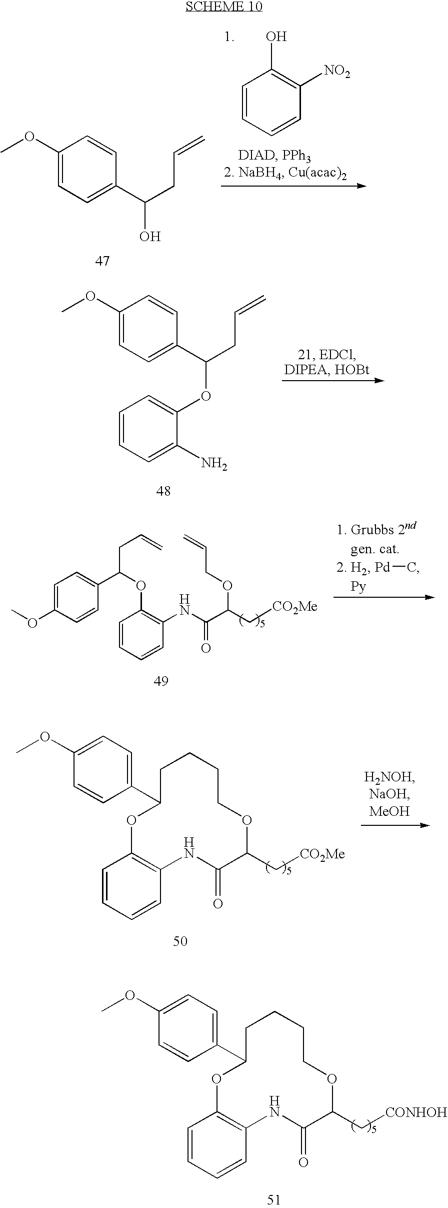

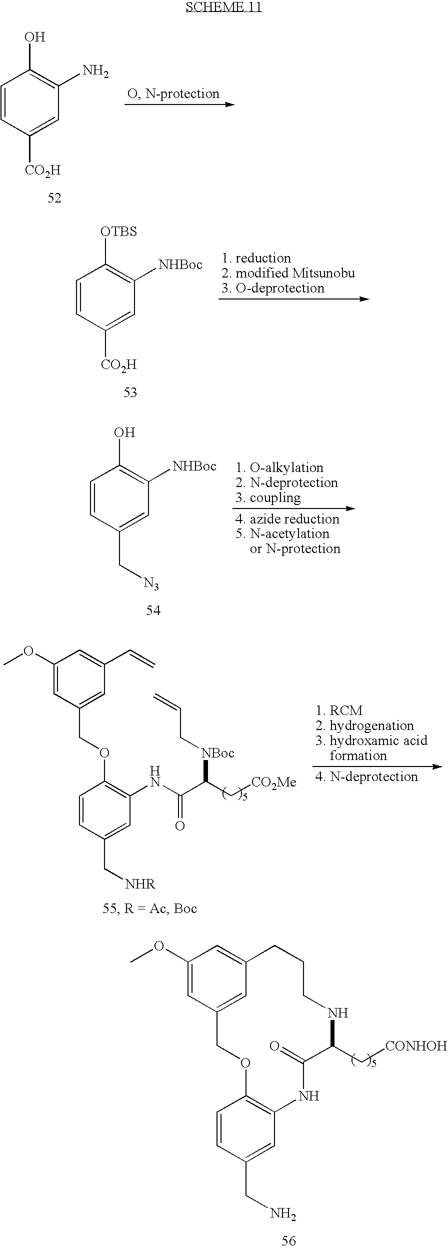

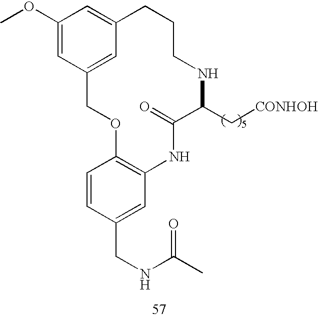

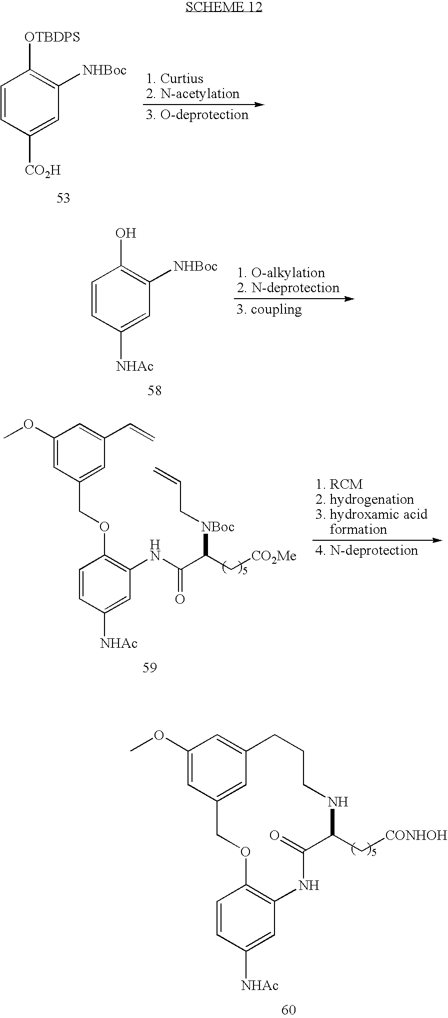

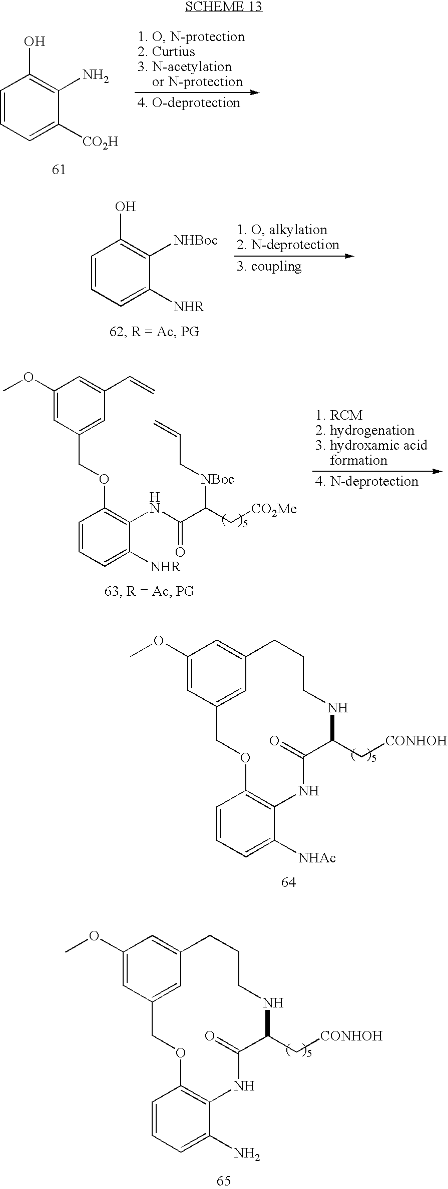

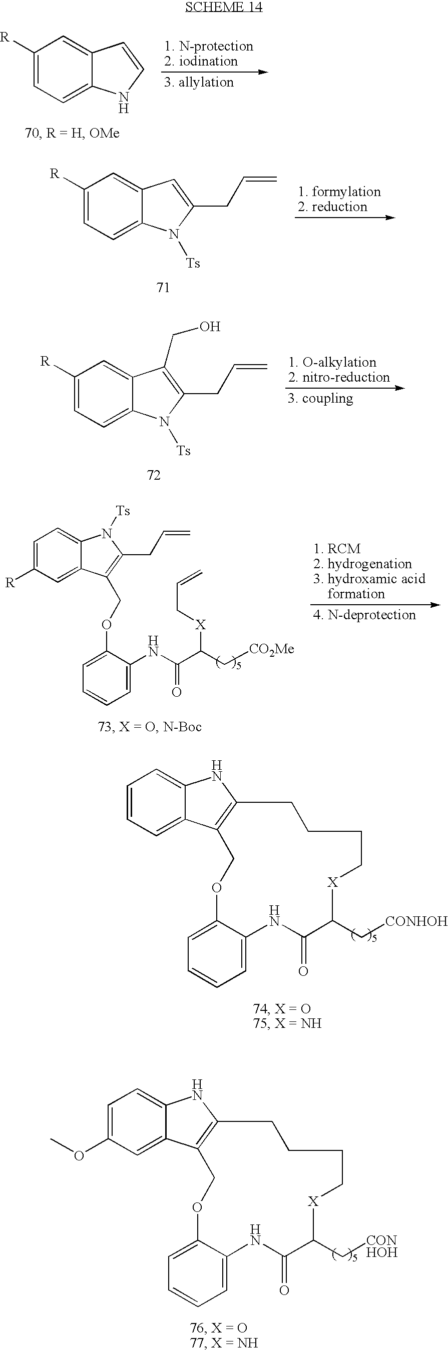

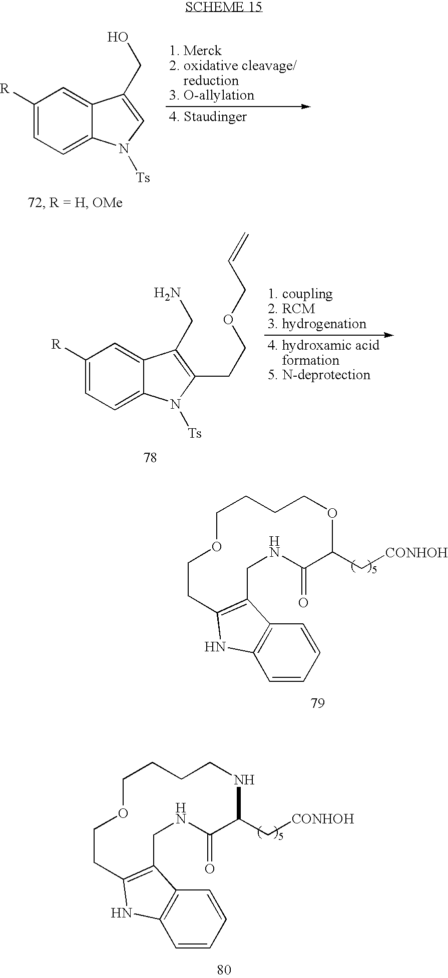

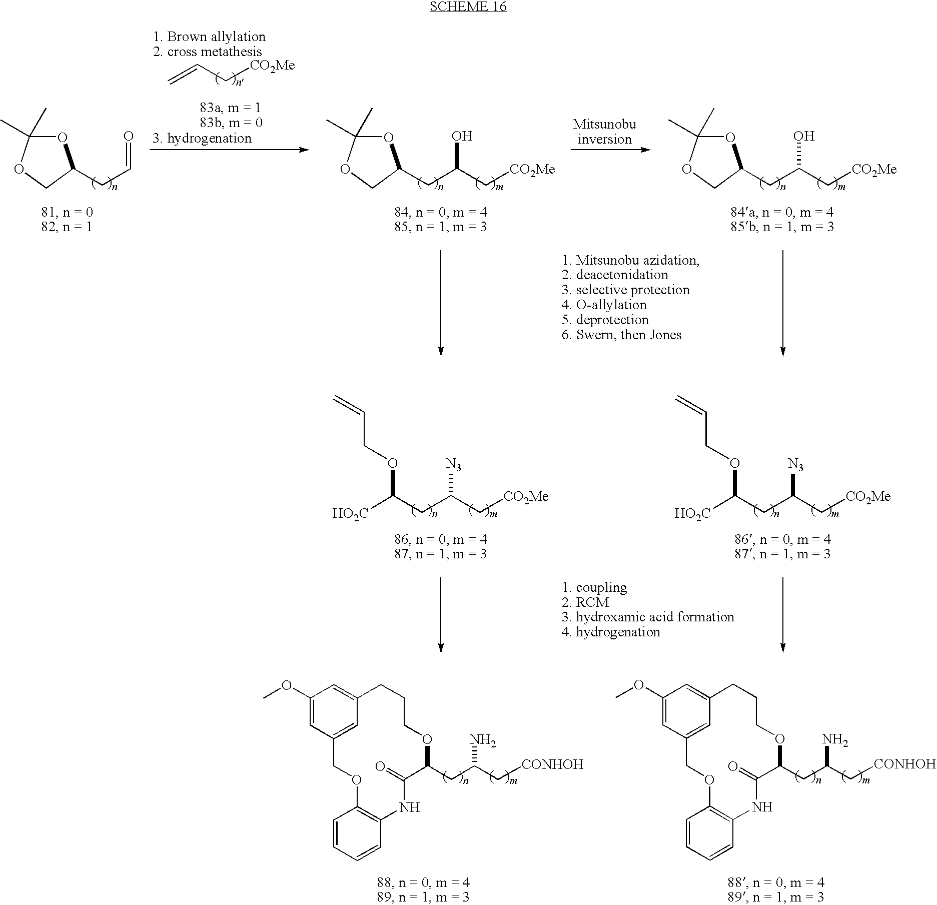

### SPOSTATO

In the following the present invention shall be illustrated by means of some examples, which are not construed to be viewed as limiting the scope of the invention.

The following abbreviations are hereinafter used in the accompanying examples: acac (acetylacetonate), Boc₂O (di-tent-butyl dicarbonate), DBU (1,8-diazabicyclo[5.4.0]undec-7-ene), DEAD (diethyl azodicarboxylate), DEBPT [3-(diethoxyphosphoryloxy)-1,2,3-benzotriazin-4(3H)-one], DIAD (di-isopropyl azodicarboxylate), DIPEA (diisopropylethylamine), DMAP (4-dimethylaminopyridine), DPPA (diphenyl phosphoryl azide), EDC [N-ethyl-N-(3-dimethylaminopropyl)carbodiimide], Grubbs' 2^{nd} generation catalyst {benzylidene[1,3-bis(2,4,6-trimethylphenyl)-2-imidazolidinylidene]dichloro(tricyclohexylphosphine)ruthenium}, KHMDS [potassium bis(trimethylsilyl)amide], LiHMDS [lithium bis(trimethylsilyl)amide], HOBt (1-hydroxybenzotriazole), PTSA (p-toluensulphonic acid), TBAF (tetrabutylammonium fluoride), TBDPS (tert-butyldiphenylsilyl), TCCA (trichloroisocyanuric acid), TEMPO (2,2,6,6-tetramethylpiperidinyl-1-oxy), TfOH (trifluoromethanesulphonic acid), aq (aqueous), sat (saturated).

Materials and Methods

All non-aqueous reactions were run in flame-dried glassware under a positive pressure of argon with exclusion of moisture from reagents and glassware using standard techniques for manipulating air-sensitive compounds Anhydrous THF, toluene, Et₂O and CH₂Cl₂ were obtained by filtration through drying columns (Solvent Delivery System); other solvents were distilled under positive pressure of dry argon before use and dried by standard methods. Unless stated otherwise, commercial grade reagents were used without further purification. Flash chromatography was performed on 230-400 mesh silica gel with the indicated solvent systems. Thin layer chromatography was performed on pre-coated, glass-backed silica gel plates (Merck 60F₂₅₄). Visualization was performed under short-wavelength ultraviolet light and/or by dipping the plates in an aqueous H₂SO₄ solution of cerium sulfate/ammonium molybdate, potassium permanganate, or ethanolic solution of anisaldehyde, followed by charring with a heat gun. Routine nuclear magnetic resonance spectra were recorded on AMX-300, ARX-400, AV-400 spectrometers (Bruker) at 400, 700, 100 and 75 MHz. Low- and high-resolution mass analyses were performed by Centre Régional de Spectroscopie de l'Université de Montréal on AEI-MS 902 or MS-50 spectrometers using electrospray (ES) techniques. Optical rotations were measured with a Perkin-Elmer 341 polarimeter at ambient temperature, using a 100 mm cell with a 1 mL capacity and are given in units of 10⁴ deg cm²g⁻¹. LCMS analyses were performed on a LC-Gilson apparatus (Autoinjector model 234, Pump 322), ThermoFinnigan LCQ Advantage MS and TSP UV6000 interface. HPLC conditions: 20-80 B %, A=H₂O, B=MeCN; Flow=0.5 mL/min; Inj. vol.=10 μL; Col. C18, 50×4.6 mm, 150×4.6 mm, or 250×4.6 mm; UV det. 214 nm, 254 nm.

### Example 1

### Preparation of Racemic 7 Carbon Long Chain ω-alkoxy Derivatives

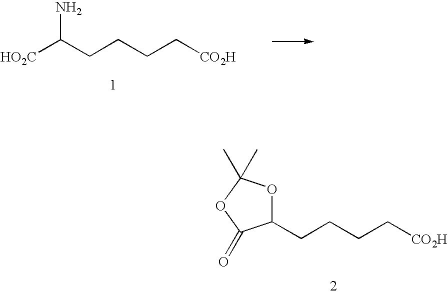

(±)-5-(2,2-Dimethyl-5-oxo-[1,3]dioxolan-4-yl)-pentanoic acid (2). To an ice-chilled solution of (±)-2-aminopimelic acid (1) (3.00 g, 17.1 mmol) in 16 mL of 2N HCl, NaNO₂ (2.36 g, 34.2 mmol) dissolved in 40 mL of H₂O was slowly added over 1 h. After 2 h at 0° C. the clear solution was stirred at room temperature overnight, and then concentrated in vacuo azeotroping off the acid with n-hexanes.

The oily residue was dissolved in H₂O (30 mL) and carefully extracted with EtOAc. The combined organic extracts were dried (MgSO₄), filtered and evaporated to give 3.0 g of a waxy solid of crude (±)-2-hydroxy-heptanedioic acid. This solid was dissolved in 2,2-dimethoxypropane (DMP) (62 mL), and was allowed to react with a catalytic amount of p-toluenesulfonic acid (PTSA) (0.23 g, 1.7 mmol) for 2 h. The reaction was quenched with H₂O and extracted with CH₂Cl₂. The combined extracts were dried (MgSO₄) and concentrated under vacuum. Flash chromatographic purification (6:4 hexanes/EtOAc) afforded acetonide 2 (2.66 g, 72% yield from 1) as a colorless oil: ¹H-NMR (CDCl₃, 300 MHz) δ 11.40 (b, 1H), 4.36 (dd, J=7.2, 4.4 Hz, 1H), 2.40 (t, J=7.2 Hz, 2H), 1.87 (m, 1H), 1.78-61 (m, 3H), 1.58 (s, 3H), 1.53-1.42 (m, 5H). ¹³C-NMR (CDCl₃, 75 MHz) δ 179.4, 173.1, 110.4, 73.7, 30.6, 31.0, 27.0, 25.6, 24.2, 24.0. MS (ESI) m/z: 217.1 (M+1), 239.1 (M+Na⁺).

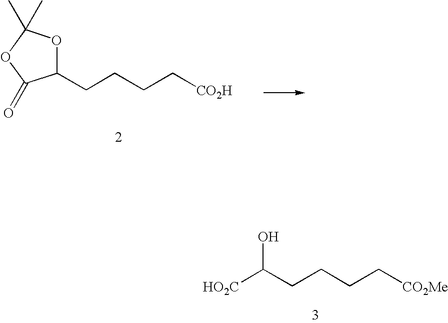

(±)-2-Hydroxy-heptanedioic acid 7-methyl ester (3). A solution of acetonide 2 (2.60 g, 12.0 mmol) in anhydrous Et₂O (10 mL) under an argon atmosphere was cooled at 0° C., and a solution of freshly distilled diazomethane in Et₂O was carefully added until the rich yellow color persisted. Stirring was continued for 30 min and the solution was allowed to warm to rt. Excess of diazomethane was destroyed by vigorous stirring and the solvent was evaporated in vacuo. The crude product was purified by flash chromatography (8:2 hexanes/EtOAc) to yield the fully protected 2-hydroxy dicarboxylic acid intermediate (2.74 g, 99%) as a colorless oil: ¹H-NMR (CDCl₃, 300 MHz) δ 4.40 (dd, J=7.2, 4.3 Hz, 1H), 3.68 (s, 3H), 2.35 (t, J=7.2 Hz, 2H), 1.91 (m, 1H), 1.81-1.65 (m, 3H), 1.61 (s, 3H), 1.57-1.45 (m, 5H). ¹³C-NMR (CDCl₃, 75 MHz) δ 174.3, 173.6, 110.9, 74.3, 52.0, 34.2, 31.6, 27.6, 26.2, 24.9 (2C). MS (ESI) m/z: 231.1 (M+1), 253.1 (M+Na⁺).

This protected intermediate (2.65 g, 11.5 mmol) was suspended in 23 mL of 70% aqueous acetic acid, and the resulting mixture was allowed to react at 60° C. The reaction was monitored by TLC, and after 2 h was quenched by addition of water (65 mL) and extracted with EtOAc. The combined extracts were dried (MgSO₄), filtered, and concentrated under vacuum to afford a crude residue that was purified by flash chromatography (100% EtOAc). Free 2-hydroxyacid 3 (1.86 g) was obtained in 85% yield as a colorless oil: ¹H-NMR (CDCl₃, 300 MHz) δ 7.80 (b, 1H), 4.29 (dd, J=7.5, 4.1 Hz, 1H), 3.69 (s, 3H), 2.37 (t, J=7.3 Hz, 2H), 2.13 (s, 1H), 1.89 (m, 1H), 1.79-1.63 (m, 3H), 1.54-1.43 (m, 2H). ¹³C-NMR (CDCl₃, 75 MHz) δ 179.5, 174.8, 70.4, 52.1, 34.2, 34.1, 24.8, 24.7. MS (ESI) m/z: 191.1 (M+1).

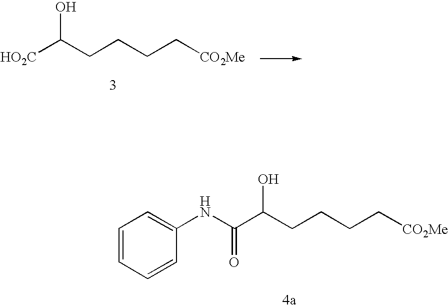

(±)-6-Hydroxy-6-phenylcarbamoyl hexanoic acid methyl ester (4a). Anilide 4a was prepared according to the general procedure (Method 1A) starting from 1.75 g of hydroxyl acid 3 (9.2 mmol), N-sulfinylaniline (Kim, Y. H.; Shin, J. M. Tetrahedron Lett. 1985, 26, 3821-3824) (1.79 g, 12.9 mmol), and 1,2,4-triazole (0.89 g, 12.9 mmol) in 13 mL of anhydrous CH₂Cl₂. After flash chromatographic purification (6:4 hexanes/EtOAc) pure anilide 4a (2.04 g) was isolated in 84% yield as a white solid: ¹H-NMR (CDCl₃, 300 MHz) δ 8.53 (b, 1H), 7.59 (d, J=7.9 Hz, 2H), 7.36 (t, J=7.7 Hz, 2H), 7.15 (t, J=7.2 Hz, 1H), 4.30 (dd, J=8.0, 3.5 Hz, 1H), 3.69 (s, 3H), 2.96 (b, 1H), 2.37 (t, J=7.2 Hz, 2H), 1.96 (m, 1H), 1.84-1.66 (m, 3H), 1.54 (m, 2H). ¹³C-NMR (CDCl₃, 75 MHz) δ 174.9, 172.2, 137.7, 129.5 (2C), 124.9, 120.1 (2C), 72.6, 52.1, 34.5, 34.1, 24.9, 24.5. MS (ESI) m/z: 266.1 (M+1), 288.1 (M+Na⁺).

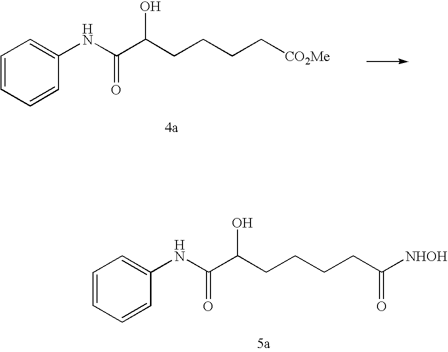

(±)-2-Hydroxyheptanedioic acid 7-hydroxyamide 1-phenylamide (5a). Hydroxamic acid 5a was prepared according to the general procedure 7A starting from the corresponding methyl ester 4a in 77% yield. A white solid: HPLC t_{R}=7.72 min. ¹H-NMR (CD₃OD, 300 MHz) δ 7.59 (d, J=8.7 Hz, 2H), 7.32 (t, J=7.5 Hz, 2H), 7.11 (t, J=7.4 Hz, 1H), 4.13 (dd, J=7.7, 4.0 Hz, 1H), 2.11 (t, J=7.3 Hz, 2H), 1.84 (m, 1H), 1.74-1.60 (m, 3H), 1.51 (m, 2H). ¹³C-NMR (CD₃OD, 75 MHz) δ 174.5, 171.8, 138.0, 128.8 (2C), 124.6, 120.5 (2C), 71.9, 34.3, 32.6, 25.5, 24.7. HRMS (ES+) C₁₃H₁₈N₂O₄ calcd for [MH]+ 267.13393. found 267.13377.

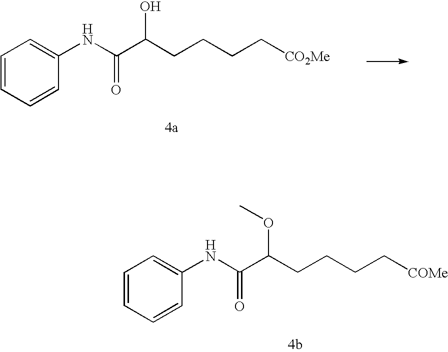

(±)-6-Methoxy-6-phenylcarbamoyl hexanoic acid methyl ester (4b). Ether 4b was prepared according to the general procedure (Method 2A) starting from alcohol 4a (250 mg, 0.94 mmol), methyl iodide (1.47 mL, 23.50 mmol) and Ag₂O (0.26 g, 1.13 mmol) in anhydrous MeCN (1.20 mL) under reflux temperature. After flash chromatography (6:4 hexanes/EtOAc) pure O-methyl ether 4b (103 mg) was recovered in 39% yield (58%, two cycles) as a colorless oil: ¹H-NMR (CDCl₃, 300 MHz) δ 8.32 (s, 1H), 7.60 (d, J=7.7 Hz, 2H), 7.37 (t, J=7.6 Hz, 2H), 7.15 (t, J=7.4 Hz, 1H), 3.77 (dd, J=6.6 Hz, 4.5 Hz, 1H), 3.68 (s, 3H), 3.50 (s, 3H), 2.35 (t, J=7.4 Hz, 2H), 1.84 (m, 2H), 1.68 (m, 2H), 1.48 (m, 2H). ¹³C-NMR (CDCl₃, 75 MHz) δ 174.4, 171.0, 137.7, 129.5 (2C), 124.9, 120.0 (2C), 82.8, 58.9, 51.9, 34.3, 32.4, 25.1, 24.7. MS (ESI) m/z: 280.2 (M+1), 302.2 (M+Na⁺).

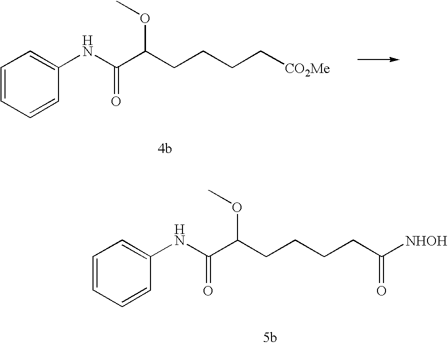

(±)-2-Methoxyheptanedioic acid 7-hydroxyamide 1-phenylamide (5b). Hydroxamic acid 5b was prepared according to the general procedure 7A starting from the corresponding methyl ester 4b in 92% yield. A colorless oil: HPLC t_{R}=8.63 min. ¹H-NMR (CD₃OD, 400 MHz) δ 7.60 (d, J=8.3 Hz, 2H), 7.34 (t, J=7.6 Hz, 2H), 7.14 (t, J=7.4 Hz, 1H), 3.78 (dd, J=6.7, 5.3 Hz, 1H), 3.44 (s, 3H), 2.12 (t, J=7.3 Hz, 2H), 1.81 (m, 2H), 1.67 (m, 2H), 1.49 (m, 2H). ¹³C-NMR (CDCl₃, 75 MHz) δ 171.8, 171.6, 137.5, 129.5 (2C), 125.1, 120.4 (2C), 82.6, 58.9, 32.8, 32.0, 25.3, 24.4. HRMS (ES+) C₁₄H₂₀N₂O₄ calcd for [MH]+ 281.14958. found 281.14944.

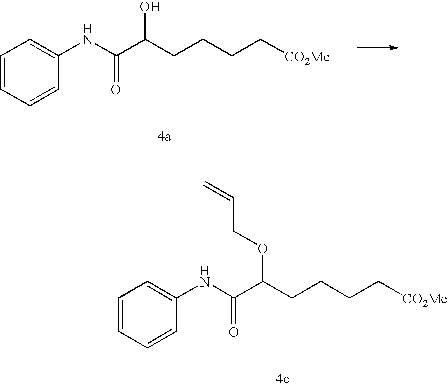

(±)-(6-Allyloxy-6-phenylcarbamoyl hexanoic acid methyl ester) (4c). Ether 4c was prepared according to the general procedure (Method 2A) starting from alcohol 4a (500 mg, 1.89 mmol), allyl bromide (4.00 mL, 47.12 mmol) and Ag₂O (4.00 mL, 47.3 mmol) in anhydrous MeCN (3.30 mL) at 45° C. After flash chromatography (7:3 hexanes/EtOAc) pure allyl ether derivative 4c (328 mg) was recovered in 66% yield as a pale yellow oil: ¹H-NMR (CDCl₃, 300 MHz) δ 8.38 (s, 1H), 7.59 (d, J=7.6 Hz, 2H), 7.36 (t, J=7.6 Hz, 2H), 7.14 (t, J=7.4 Hz, 1H), 5.98 (ddt, J=17.2, 10.4, 5.7 Hz, 1H), 5.38, (dd, J=17.3, 1.5 Hz, 1H), 5.29 (dd, J=10.4, 1.3 Hz, 1H), 4.12 (dt, J=5.7, 1.3 Hz, 2H), 3.93 (dd, J=6.8, 4.6 Hz, 1H), 3.68 (s, 3H), 2.34 (t, J=7.9 Hz, 2H), 1.85 (m, 2H), 1.69 (m, 2H), 1.50 (m, 2H). ¹³C-NMR (CDCl₃, 75 MHz) δ 174.4, 171.2, 137.7, 134.0, 129.5 (2C), 124.9, 120.0 (2C), 118.8, 80.5, 72.2, 52.0, 34.3, 32.9, 25.1, 25.0. MS (ESI) m/z: 306.2 (M+1), 328.2 (M+Na⁺).

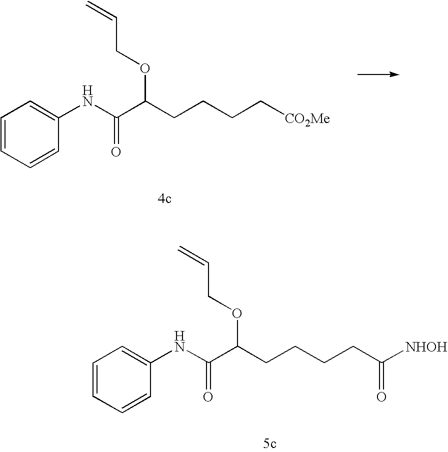

(±)-2-Allyloxyheptanedioic acid 7-hydroxyamide 1-phenylamide (5c). Hydroxamic acid 5c was prepared according to the general procedure 7A starting from the corresponding methyl ester 4c in 77% yield. A colorless oil: HPLC t_{R} 9.98 min. ¹H-NMR (CD₃OD, 400 MHz) δ 7.60 (d, J=8.2 Hz, 2H), 7.34 (t, J=8.0 Hz, 2H), 7.14 (t, J=7.4 Hz, 1H), 6.00 (ddt, J=17.0, 10.8, 5.9 Hz, 1H), 5.35 (d, J=17.2 Hz, 1H), 5.24 (d, J=10.4 Hz, 1H), 4.18 (dd, J=12.8, 5.5 Hz, 1H), 4.04 (dd, J=12.7, 5.9 Hz, 1H), 3.94 (t, J=6.1, Hz, 1H), 2.12 (t, J=7.2 Hz, 2H), 1.81 (m, 2H), 1.68 (m, 2H), 1.51 (m, 2H). ¹³C-NMR (CDCl₃, 75 MHz) δ 171.8 (2C), 137.5, 134.0, 129.5 (2C), 125.1, 120.3 (2C), 118.9, 80.2, 72.2, 32.8, 32.5, 25.3, 24.6. HRMS (ES+) C₁₆H₂₂N₂O₄ calcd for [MH]+ 307.16523. found 307.16488.

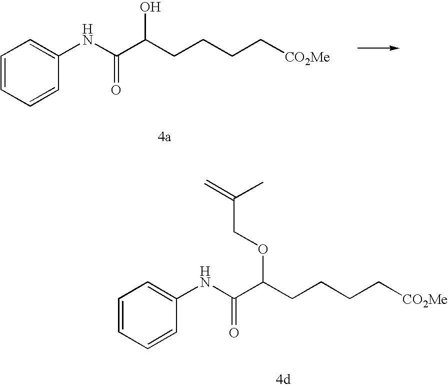

(±)-6-(2-Methylallyloxy)-6-phenylcarbamoyl hexanoic acid methyl ester 4d). Ether 4d was prepared according to the general procedure (Method 2A) starting from alcohol 4a (250 mg, 0.94 mmol), 3-bromo-2-methylpropene (2.37 mL, 23.50 mmol) and Ag₂O (0.26 g, 1.13 mmol) in anhydrous MeCN (1.65 mL). After flash chromatographic purification (7:3 hexanes/EtOAc) pure 2-methylallyl ether 4d (100 mg) was isolated in 38% yield as a colorless oil: ¹H-NMR (CDCl₃, 300 MHz) δ 8.40 (s, 1H), 7.59 (d, J=8.5, 1.2 Hz, 2H), 7.36 (t, J=7.6 Hz, 2H), 7.14 (t, J=7.4 Hz, 1H), 5.04 (d, J=19.8 Hz, 2H), 4.01 (s, 2H), 3.92 (dd, J=6.4, 4.9 Hz, 1H), 3.67 (s, 3H), 2.34 (t, J=7.4 Hz, 2H), 1.83, (m, 2H), 1.82 (s, 3H), 1.67 (m, 2H), 1.51 (m, 2H). ¹³C-NMR (CDCl₃, 75 MHz) δ 174.4, 171.2, 141.6, 137.7, 129.5 (2C), 124.9, 120.0 (2C), 113.7, 80.4, 75.1, 52.0, 34.3, 32.8, 25.1, 24.7, 20.1. MS (ESI) m/z: 320.2 (M+1), 342.2 (M+Na⁺).

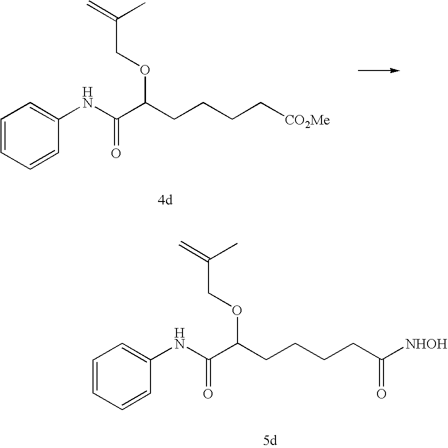

(±)-2-(2-Methylallyloxy)heptanedioic acid 7-hydroxyamide 1-phenylamide (5d). Hydroxamic acid 5d was prepared according to the general procedure 7A starting from the corresponding methyl ester 4d in 75% yield. A pale yellow oil: HPLC t_{R}=10.85 min. ¹H-NMR (CD₃OD, 400 MHz) δ 7.60 (d, J=8.1 Hz, 2H), 7.34 (t, J=7.6 Hz, 2H), 7.14 (t, J=7.5 Hz, 1H), 5.04 (s, 1H), 4.97 (s, 1H), 4.09 (d, J=12.4 Hz, 1H), 3.93 (m, 2H), 2.12 (t, J=7.3, 2H), 1.89-1.80 (m, 5H), 1.68 (m, 2H), 1.52 (m, 2H). ¹³C-NMR (CDCl₃, 75 MHz) δ 171.7, 171.5, 141.5, 137.5, 129.5 (2C), 125.1, 120.2 (2C), 113.9, 80.2, 75.0, 32.8, 32.4, 25.3, 24.6, 20.1. HRMS (ES+) C₁₇H₂₄N₂O₄ calcd for [MH]+ 321.18088. found 321.18122.

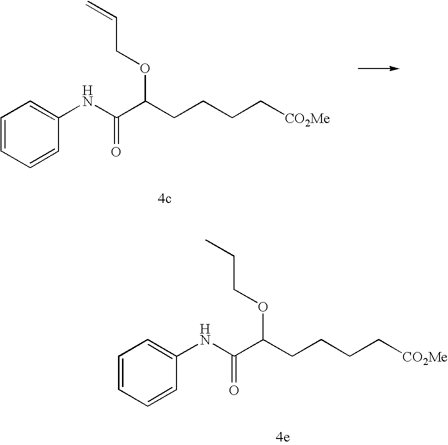

(±)-6-Phenylcarbamoyl-6-propoxy hexanoic acid methyl ester (4e). O-allyl intermediate 4c (180 mg, 0.59 mmol) was dissolved in MeOH (4.0 mL) and catalytic 10% palladium on carbon was added under stirring at rt. The reaction vessel was evacuated by aspiration and thoroughly purged with H₂ (three times) and the resulting heterogeneous mixture was stirred under a balloon of H₂. After 24 h the H₂ was evacuated, the catalyst filtered off, and the filtrate concentrated under reduced pressure to give a crude residue. After flash chromatographic purification (7:3 hexanes/EtOAc) pure n-propyl ether 4e (145 mg) was recovered in 80% yield as a colorless oil: ¹H-NMR (CDCl₃, 300 MHz) δ 8.39 (s, 1H), 7.59 (dd, J=8.3, 1.2 Hz, 2H), 7.37 (t, J=7.5 Hz, 2H), 7.15 (t, J=7.4, 1H), 3.84 (dd, J=7.1, 4.4 Hz, 1H), 3.68 (s, 3H), 3.55 (t, J=6.5 Hz, 2H), 2.35 (t, J=7.8 Hz, 2H), 1.85 (m, 2H), 1.91-1.64 (m, 4H), 1.50 (m, 2H), 1.04 (t, J=7.4 Hz, 3H). ¹³C-NMR (CDCl₃, 75 MHz) δ 174.4, 171.6, 137.5, 120.5 (2C), 124.8, 119.9 (2C), 81.2, 73.3, 52.0, 34.3, 32.9, 25.1, 25.0, 23.5, 11.2. MS (ESI) m/z: 308.2 (M+1), 330.2 (M+Na⁺).

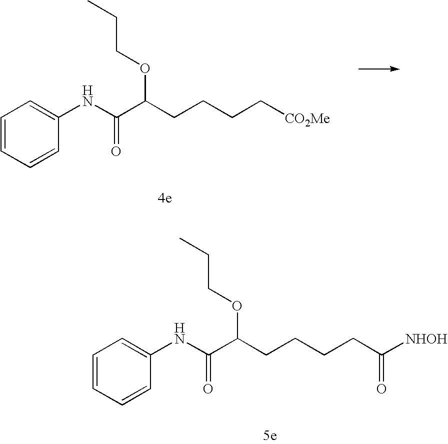

(±)-2-Propoxyheptanedioic acid 7-hydroxyamide 1-phenylamide (5e). Hydroxamic acid 5e was prepared according to the general procedure 7A starting from the corresponding methyl ester 4e in 98% yield. A colorless oil: HPLC t_{R}=10.48 min. ¹H-NMR (CD₃OD, 400 MHz) δ 7.59 (d, J=8.1 Hz, 2H), 7.34 (t, J=7.5 Hz, 2H), 7.14 (t, J=7.4 Hz, 1H), 3.86 (t, J=6.2 Hz, 1H), 3.57 (m, 1H), 3.45 (m, 1H), 2.12 (t, J=7.3 Hz, 2H), 1.80 (m, 2H), 1.74-1.65 (m, 4H), 1.50 (m, 2H), 1.00 (t, J=7.4 Hz, 3H). ¹³C-NMR (CDCl₃, 75 MHz) δ 172.1, 171.7, 137.5, 129.5 (2C), 125.0, 120.19 (2C), 81.0, 73.3, 32.8, 32.6, 25.4, 24.7, 23.5, 11.2. HRMS (ES+) C₁₆H₂₄N₂O₄ calcd for [MH]+ 309.18088. found 309.18159.

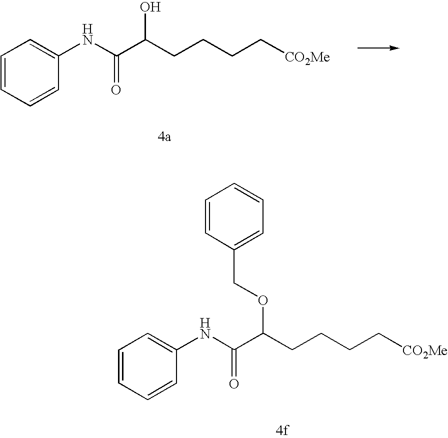

(±)-6-Benzyloxy-6-phenylcarbamoyl hexanoic acid methyl ester (40. Ether 4f was prepared according to the general procedure (Method 2A) starting from alcohol 4a (250 mg, 0.94 mmol), benzyl bromide (1.12 mL, 9.40 mmol) and Ag₂O (261 mg, 1.13 mmol) in anhydrous DMF (1.20 mL). After flash chromatographic purification (7:3 hexanes/EtOAc) pure 0-benzyl ether 4f (135 mg) was isolated in 40% yield as a pale yellow oil: ¹H-NMR (CDCl₃, 300 MHz) δ 8.40 (s, 1H), 7.57-7.54 (m, 2H), 7.45-7.29 (m, 7H), 7.15 (t, J=7.4 Hz, 1H), 4.65 (s, 2H), 4.01 (dd, J=6.4, 4.5 Hz, 1H), 3.68 (s, 3H), 2.34 (t, J=6.7 Hz, 2H), 1.86 (m, 2H), 1.67 (m, 2H), 1.51 (m, 2H). ¹³C-NMR (CDCl₃, 75 MHz) δ 174.4, 171.2, 137.7, 137.3, 129.5, 129.2 (2C), 128.8 (2C), 128.6 (2C), 124.9, 120.0 (2C), 80.7, 73.5, 52.0, 34.3, 32.9, 25.1, 24.9. MS (ESI) m/z: 356.3 (M+1).

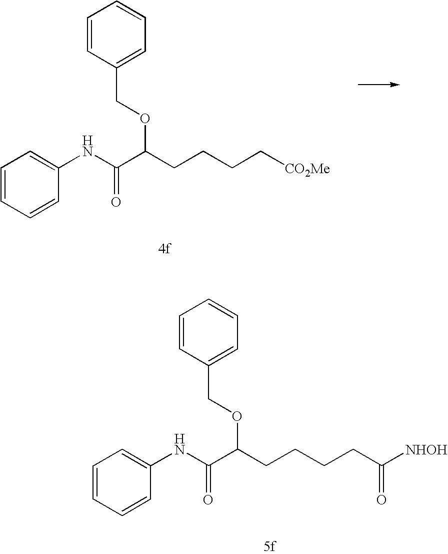

(±)-2-Benzyloxyheptanedioic acid 7-hydroxyamide 1-phenylamide (5t). Hydroxamic acid 5f was prepared according to the general procedure 7A starting from the corresponding methyl ester 4f in 73% yield. A pale yellow oil: HPLC t_{R}=11.57 min. ¹H-NMR (CD₃OD, 400 MHz) δ 7.58 (d, J=7.6 Hz, 2H), 7.43-7.28 (m, 7H) 7.14 (t, J=7.4 Hz, 1H), 4.70 (d, J=11.8 Hz, 1H), 4.54 (d, J=11.8 Hz, 1H), 3.97 (t, J=5.7 Hz, 1H), 2.10 (t, J=7.2 Hz, 2H), 1.82 (m, 2H), 1.63 (m, 2H), 1.49 (m, 2H). ¹³C-NMR (CDCl₃, 75 MHz) δ 171.6 (2C), 137.4, 137.3, 129.5, 129.2 (2C), 128.9 (2C), 128.6 (2C), 125.1, 120.3 (2C), 80.5, 73.5, 32.4, 32.0, 25.2, 24.5. HRMS (ES+) C₂₀H₂₄N₂O₄ calcd for [MH]+ 357.18088. found 357.18049.

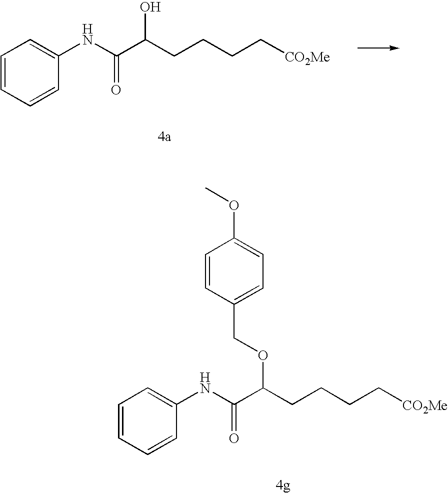

(±)-6-(4-Methoxybenzyloxy)-6-phenylcarbamoyl hexanoic acid methyl ester (4g). Ether 4g was prepared according to the general procedure (Method 2A) starting from alcohol 4a (300 mg, 1.13 mmol) p-methoxybenzyl bromide (a freshly prepared 2 M solution in toluene, 5.65 mL) and Ag₂O (311 mg, 1.34 mmol) at 45° C. After flash chromatographic purification (gradient 9:1 to 6:4 hexanes/EtOAc) pure p-methoxybenzyl ether 4g (131 mg) was isolated in 30% yield as a pale yellow oil: ¹H-NMR (CDCl₃, 300 MHz) δ 8.40 (s, 1H), 7.55 (dd, J=8.4, 1.2 Hz, 2H), 7.32 (t, J=7.5 Hz, 2H), 7.32 (d, J=8.6 Hz, 2H), 7.15 (t, J=7.4 Hz, 1H), 6.93 (d, J=9.2 Hz, 2H), 4.57 (s, 2H), 3.98 (dd, J=7.0, 4.5 Hz, 1H), 3.83 (s, 3H), 3.68 (s, 3H), 2.32 (t, J=7.2 Hz, 2H), 1.85 (m, 2H), 1.66 (m, 2H), 1.50 (m, 2H). ¹³C-NMR (CDCl₃, 75 MHz) δ 174.4, 171.4, 160.2, 137.7, 130.3 (2C), 129.5 (2C), 129.4, 124.9, 120.0 (2C), 114.6 (2C), 80.4, 73.3, 55.8, 52.0, 34.3, 32.9, 25.0, 24.9. MS (ESI) m/z: 386.1 (M+1).

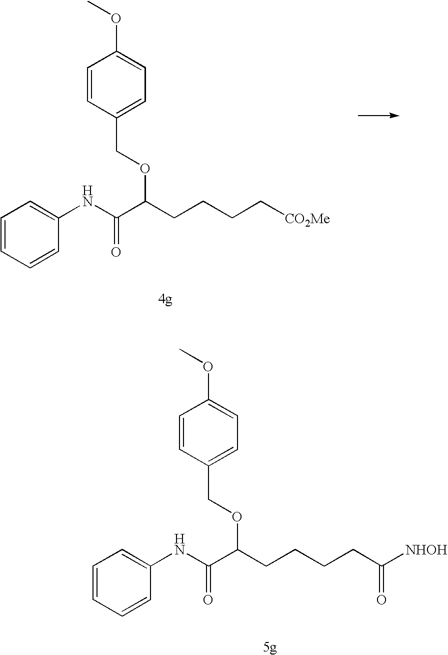

(±)-2-(4-Methoxybenzyloxy)heptanedioic acid 7-hydroxyamide 1-phenylamide (5g). Hydroxamic acid 5g was prepared according to the general procedure 7A starting from the corresponding methyl ester 4g in 65% yield. A pale yellow oil: HPLC t_{R}=11.51 min. ¹H-NMR (CD₃OD, 400 MHz) δ 7.56 (d, J=8.2 Hz, 2H), 7.35-7.31 (m, 4H), 7.14 (t, J=7.5 Hz, 1H), 6.92 (d, J=8.5 Hz, 2H), 4.63 (d, J=11.5 Hz, 1H), 4.48 (d, J=11.5 Hz, 1H), 3.94 (t, J=6.0 Hz, 1H), 3.79 (s, 3H), 2.09 (t, J=7.3, 2H), 1.79 (m, 2H), 1.63 (m, 2H), 1.48 (m, 2H). ¹³C-NMR (CDCl₃, 75 MHz) δ 171.8 (2C), 160.2, 137.5, 130.3 (2C), 129.5 (2C), 129.3, 125.1, 120.2 (2C), 114.6 (2C), 80.1, 73.2, 55.8, 32.7, 32.5, 25.2, 24.6. HRMS (ES+) C₂₁H₂₆N₂O₅ calcd for [MH]+ 387.19145. found 387.18975.

### Example 2

### Preparation of Racemic 8 Carbon Long Chain ω-Alkoxy Derivatives

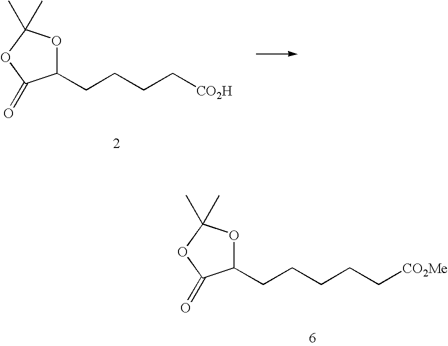

(±)-6-(2,2-Dimethyl-5-oxo-[1,3]dioxolan-4-yl)hexanoic acid methyl ester (6). To a solution of partially protected carboxylic acid 2 (0.50 g scale reaction, 2.31 mmol) in CH₂Cl₂ (13 mL) under argon, SOCl₂ (1.67 mL, 23.10 mmol) and a catalytic amount of anhydrous DMF (36 μL, 0.46 mmol) were added at −10° C. The resulting solution was allowed to raise room temperature while stirring for 30 min.

Solvent and excess of SOCl₂ were removed under reduced pressure and the residue was dissolved in anhydrous Et₂O (2.5 mL). After cooling at −50° C. a freshly distilled solution of diazomethane in Et₂O was carefully added to the white suspension under vigorous stirring until the rich yellow color persisted. Stirring was continued for 3 h as the mixture was allowed to warm rt. After removal of the excess of diazomethane by vigorous stirring, the solvent was removed under reduced pressure and the resulting crude product purified by flash chromatography (7:3 hexanes/EtOAc). Diazoketone intermediate (478 mg) was thus obtained in 86% yield (calculated from 2) as a yellow oil: ¹H-NMR (CDCl₃, 300 MHz) δ 5.27 (s, 1H), 4.40 (dd, J=7.0, 4.4 Hz, 1H), 2.35 (m, 2H), 1.89 (m, 1H), 1.78-1.66 (m, 3H), 1.60 (s, 3H), 1.54 (s, 3H), 1.55-1.46 (m, 2H). MS (ESI) m/z: 241.1 (M+1), 263.1 (M+Na⁺).

This diazoketone intermediate (0.40 mg, 1.67 mmol) and Et₃N (0.47 mL, 3.34 mL) were dissolved in anhydrous MeOH (11.30 mL), and then cooled to −25° C. under an argon atmosphere with the exclusion of the light. Silver benzoate (38 mg, 0.17 mmol) was slowly added in portions and the resulting mixture was allowed to warm to room temperature in a period of 1 h. Once room temperature was reached the reaction was immediately quenched with NH₄Cl (aq, sat.) and extracted with CH₂Cl₂. The organic phase was dried (MgSO₄), evaporated and purified by flash chromatography (75:25 hexanes/EtOAc) which gave 0.40 g (99% yield) of rearranged methyl ester 6 as a colorless oil: ¹H-NMR (CDCl₃, 300 MHz) δ 4.40 (dd, J=7.0, 4.4 Hz, 1H), 3.68 (s, 3H), 2.33 (t, J=7.4 Hz, 1H), 1.87 (m, 1H), 1.76-1.61 (m, 3H), 1.62 (s, 3H), 1.55 (s, 3H), 1.52-1.35 (m, 5H). ¹³C-NMR (CDCl₃, 75 MHz) δ 174.5, 173.7, 110.8, 74.4, 51.9, 34.3, 31.7, 29.1, 27.6, 26.2, 25.1, 24.9. MS (ESI) m/z: 245.1 (M+1).

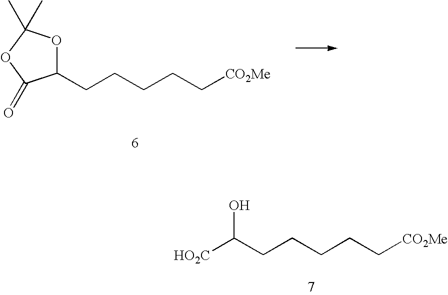

(±)-2-Hydroxyoctanedioic acid 8-methyl ester (7). Fully protected hydroxy diacid 6 (1.20 g, 4.9 mmol) was suspended in 10.0 mL of 70% aqueous acetic acid, and stirred at 60° C. After 2 h at this temperature the reaction was judged complete (monitoring by TLC), and was quenched by addition of water (30 mL) and extracted with EtOAc. The combined extracts were dried (MgSO₄), filtered and concentrated under vacuum to afford partially deprotected 2-hydroxy acid 7 (0.98 g, 98%) which was used for the next reaction without further purification. A colorless oil: ¹H-NMR (CDCl₃, 300 MHz) δ: 7.50 (b, 1H), 4.29 (dd, J=7.4, 4.2 Hz, 1H), 3.69 (s, 3H), 2.35 (t, J=7.4 Hz, 2H), 2.14 (s, 1H), 1.87 (m, 1H), 1.79-1.60 (m, 3H), 1.57-1.33 (m, 4H). ¹³C-NMR (CDCl₃, 75 MHz) δ: 179.8, 175.0, 70.5, 52.1, 34.3, 34.2, 29.1, 25.1, 24.8. MS (ESI) m/z: 205.1 (M+1), 227.1 (M+Na⁺).

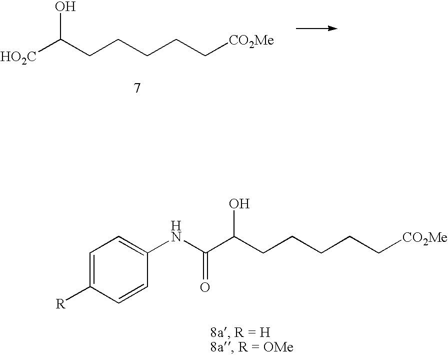

(±)-7-Hydroxy-7-phenylcarbamoylheptanoic acid methyl ester (8a′) and (±)-7-Hydroxy-7-(4-methoxyphenylcarbamoyl)heptanoic acid methyl ester (8a″) Anilide 8a′ was prepared according to the general procedure (Method 1A), starting from 2-hydroxy acid 7 (0.90 g, 4.4 mmol), N-sulfinylaniline (0.86 g, 6.16 mmol) and 1,2,4-triazole (0.43 g, 6.16 mmol) in CH₂Cl₂ (6.0 mL). After flash chromatography (gradient 7:3 to 1:1 hexanes/EtOAc) pure 8a′ (1.06 g) was recovered in 86% yield as a pale yellow solid: ¹H-NMR (CDCl₃, 300 MHz) δ 8.50 (s, 1H), 7.59 (d, J=8.1 Hz, 2H), 7.36 (t, J=7.7 Hz, 2H), 7.15 (t, J=7.1 Hz, 1H), 4.27 (dd, J=7.6, 3.6 Hz, 1H), 3.69 (s, 3H), 2.95 (b, 1H), 2.35 (t, J=7.4, 2H), 1.95 (m, 1H), 1.83-1.62 (m, 3H), 1.57-1.40 (m, 4H). ¹³C-NMR (CDCl₃, 75 MHz) δ 174.9, 172.2, 137.7, 129.5 (2C), 124.9, 120.4 (2C), 72.8, 52.0, 34.9, 34.3, 29.0, 25.0, 24.9. MS (ESI) m/z: 280.1 (M+1), 302.1 (M+Na⁺).

Anilide 8a″ was prepared according to the general procedure (Method 1A) from 2-hydroxy acid 7 (0.90 g, 4.4 mmol), N-sulfinylanisidine (1.05 g, 6.16 mmol) and 1,2,4-triazole (0.43 g, 6.16 mmol) in CH₂Cl₂ (6.0 mL). After flash chromatography (gradient 7:3 to 1:1 hexanes/EtOAc) pure 8a″ (0.98 g) was recovered in 72% yield as an amorphous yellow solid: ¹H-NMR (CDCl₃, 400 MHz) δ 8.40 (s, 1H), 7.48 (d, J=6.8 Hz, 2H), 6.88 (d, J=9.0 Hz, 2H), 4.23 (dd, J=7.8, 3.7 Hz, 1H), 3.81 (s, 3H), 3.68 (s, 3H), 2.71 (b, 1H), 2.34 (t, J=7.4 Hz, 2H), 1.93 (m, 1H), 1.74 (m, 1H), 1.66 (m, 2H), 1.50 (m, 2H), 1.37 (m, 2H). ³C-NMR (CDCl₃, 100 MHz) δ 174.1, 171.3, 156.1, 130.0, 121.1 (2C), 113.8 (2C), 71.9, 55.1, 51.2, 34.1, 33.5, 26.3, 24.2 (2C). MS (ESI) m/z: 310.1.

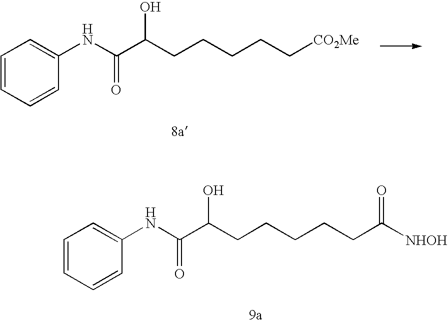

(±)-2-Hydroxyoctanedioic acid 8-hydroxyamide 1-phenylamide (9a). Hydroxamic acid 9a was prepared according to the general procedure 7A starting from the corresponding methyl ester 8a′ in 70% yield. A white solid: HPLC t_{R}=3.81 min. ¹H-NMR (CD₃OD, 300 MHz) δ 7.58 (d, J=7.6 Hz, 2H), 7.32 (t, J=7.5 Hz, 2H), 7.11, (t, J=7.5 Hz, 1H), 4.13 (dd, J=7.7, 4.0 Hz, 1H), 2.09 (t, J=7.5 Hz, 2H), 1.82 (m, 1H), 1.72-1.58 (m, 3H), 1.55-32 (m, 4H). ¹³C-NMR (CD₃OD, 75 MHz) δ 174.6, 172.0, 138.0, 128.8 (2C), 124.6, 120.5 (2C), 72.1, 34.6, 32.7, 28.9, 25.7, 24.8. HRMS (ES+) C₁₄H₂₀N₂O₄ calcd for [MH]+ 281.14958. found 281.14967.

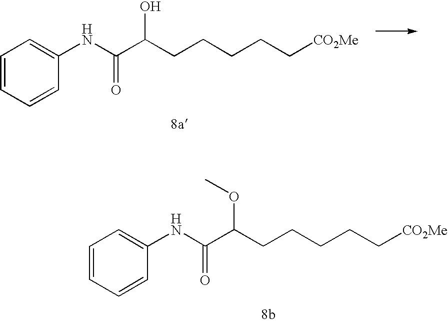

(±)-7-Methoxy-7-phenylcarbamoyl heptanoic acid methyl ester (8b). Ether 8b was prepared according to the general procedure (Method 2A) starting from alcohol 8a′ (250 mg, 0.90 mmol) methyl iodide (1.40 mL, 22.50 mmol) and Ag₂O (0.25 g, 1.08 mmol) in anhydrous MeCN (1.20 mL) under reflux temperature. After purification by flash chromatography (gradient 7:3 to 1:1 hexanes/EtOAc) pure O-methyl ether 8b (197 mg) was recovered in 74% yield as a colorless oil: ¹H-NMR (CDCl₃, 300 MHz) δ 8.32 (s, 1H), 7.60 (dd, J=8.4, 0.9 Hz, 2H), 7.36 (t, J=7.5 Hz, 2H), 7.15 (t, J=7.4 Hz, 1H), 3.77 (dd, J=6.6, 4.6 Hz, 1H), 3.68 (s, 3H), 3.50 (s, 3H), 2.33 (t, J=7.4 Hz, 2H), 1.85 (m, 2H), 1.66 (m, 2H), 1.49-1.31 (m, 4H). ¹³C-NMR (CDCl₃, 75 MHz) δ 174.6, 171.2, 137.7, 129.5 (2C), 124.8, 120.0 (2C), 83.0, 58.9, 51.9, 34.4, 32.6, 29.3, 25.2, 24.8. MS (ESI) m/z: 294.2 (M+1), 316.2 (M+Na⁺).

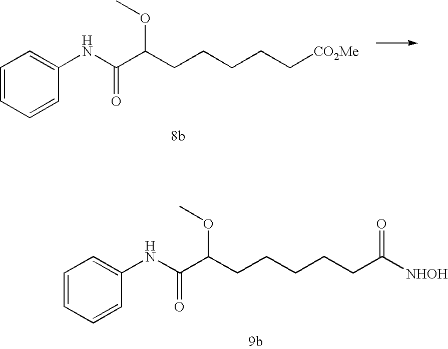

(±)-2-Methoxyoctanedioic acid 8-hydroxyamide 1-phenylamide (9b). Hydroxamic acid 9b was prepared according to the general procedure 7A starting from the corresponding methyl ester 8b in 79% yield. A colorless oil (79% yield): HPLC t_{R}=4.39 min. ¹H-NMR (CD₃OD, 400 MHz) δ 7.61 (d, J=7.7 Hz, 2H), 7.34 (t, J=7.6 Hz, 2H), 7.14 (t, J=7.4 Hz, 1H), 3.77 (t, J=6.0 Hz, 1H), 3.45 (s, 3H), 2.10 (t, J=7.3 Hz, 2H), 1.79 (m, 2H), 1.64 (m, 2H), 1.48 (m, 2H), 1.39 (m, 2H). ¹³C-NMR (CDCl₃, 75 MHz) δ 171.9, 171.7, 137.5, 129.5 (2C), 125.1, 120.3 (2C), 82.7, 58.9, 32.9, 32.4, 28.8, 25.4, 24.4. HRMS (ES+) C₁₅H₂₂N₂O₄ calcd for [MH]⁺ 295.16523. found 295.16582.

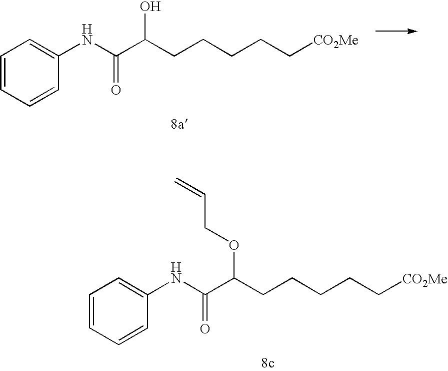

(±)-7-Allyloxy-7-phenylcarbamoyl heptanoic acid methyl ester (8c). Ether 8c was prepared according to the general procedure (Method 2A) starting from alcohol 8a′ (250 mg, 0.90 mmol), allyl iodide (2.05 mL, 22.50 mmol) and Ag₂O (0.25 g, 1.08 mmol) in anhydrous MeCN (1.4 mL) at 45° C. Purification by flash chromatography (gradient 9:1 to 6:4 hexanes/EtOAc) afforded pure O-allyl ether 8c (230 mg) in 80% yield as a pale yellow oil: ¹H-NMR (CDCl₃, 300 MHz) δ 8.38 (s, 1H), 7.59 (dd, J=8.5, 1.0 Hz, 2H), 7.36 (t, J=7.6 Hz, 2H), 7.15 (t, J=7.4, 1H), 5.96 (ddt, J=17.2, 10.4, 5.7 Hz, 1H), 5.37 (dd, J=17.2, 1.6 Hz, 1H), 5.30 (ddt, J=10.4, 1.4 Hz, 1H), 4.13 (dt, J=5.6, 1.3 Hz, 2H), 3.92 (dd, J=6.8, 4.6 Hz, 1H), 3.68 (s, 3H), 2.33 (t, J=7.4 Hz, 2H), 1.84, (m, 2H), 1.66 (m, 2H), 1.56-1.32 (m, 4H). ¹³C-NMR (CDCl₃, 100 MHz) δ 174.6, 171.4, 137.7, 134.1, 129.5 (2C), 124.9, 120.0 (2C), 118.7, 80.6, 72.2, 52.0, 34.4, 33.1, 29.3, 25.2, 25.0. MS (ESI) m/z: 320.3 (M+1), 342.3 (M+Na⁺).

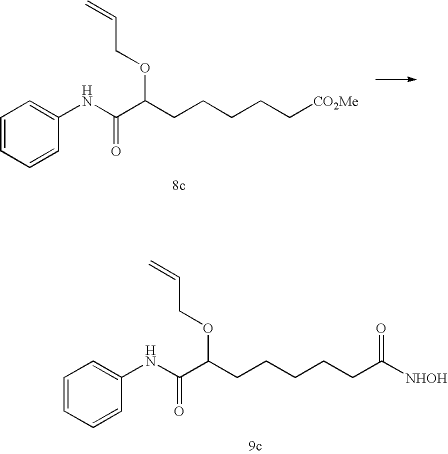

(±)-2-Allyloxyoctanedioic acid 8-hydroxyamide 1-phenylamide (9c). Hydroxamic acid 9c was prepared according to the general procedure 7A starting from the corresponding methyl ester 8c in 98% yield. A colorless oil: HPLC t_{R}=5.17 min. ¹H-NMR (CD₃OD, 400 MHz) δ 7.59 (d, J=7.6 Hz, 2H), 7.34 (t, J=7.6 Hz, 2H), 7.14 (t, J=7.4 Hz, 1H), 6.00 (ddt, J=17.1, 10.5, 5.8 Hz, 1H), 5.35 (dd, J=17.2, 1.5 Hz, 1H), 5.24 (dd, J=10.4, 1.2 Hz, 1H), 4.18 (dd, J=12.8, 5.6 Hz, 1H), 4.03 (dd, J=12.8, 6.0 Hz, 1H), 3.93 (t, J=6.1 Hz, 1H), 2.11 (t, J=7.3 Hz, 2H), 1.80 (m, 2H), 1.65 (m, 2H), 1.50 (m, 2H), 1.39 (m, 2H). ¹³C-NMR (CDCl₃, 75 MHz) δ 171.8 (2C), 137.5, 134.0, 129.5 (2C), 125.0, 120.2 (2C), 118.9, 80.3, 72.2, 33.0, 32.8, 28.8, 25.4, 24.6. HRMS (ES+) C₁₇H₂₄N₂O₄ calcd for [MH]⁺ 321.18088, found 321.18094.

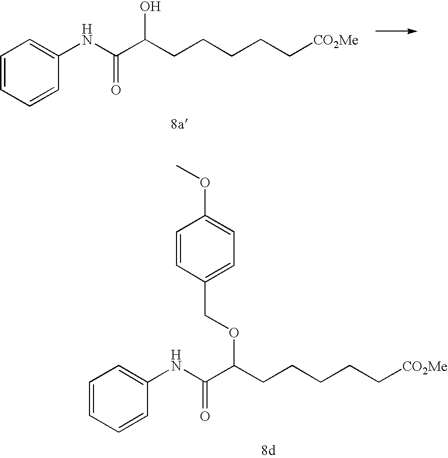

(±)-7-(4-Methoxybenzyloxy)-7-phenylcarbamoyl heptanoic acid methyl ester (8d). Ether 8d was prepared according to the general procedure (Method 2B) starting from 250 mg of alcohol 8a′ (0.90 mmol) in 1.3 mL of Et₂O in the presence of catalytic BF₃.Et₂O (1 μL, 9×10⁻³ mmol). After purification by flash chromatography (gradient 9:1 to 7:3 hexanes/EtOAc) pure p-methoxybenzyl ether 8d (140 mg) was recovered in 39% yield as a pale yellow oil: ¹H-NMR (CDCl₃, 400 MHz) δ 8.39 (s, 1H), 7.55 (d, J=7.6 Hz, 2H), 7.35 (t, J=7.6 Hz, 2H), 7.31 (d, J=8.7 Hz, 2H), 7.14 (t, J=7.4 Hz, 1H), 6.93 (d, J=9.0 Hz, 2H), 4.57 (s, 2H), 3.97 (dd, J=7.1, 4.4 Hz, 1H), 3.84 (s, 3H), 3.68 (s, 3H), 2.31 (t, J=7.4 Hz, 2H), 1.83 (m, 2H), 1.63 (m, 2H), 1.45 (m, 2H), 1.32 (m, 2H). ¹³C-NMR (CDCl₃, 100 MHz) δ 173.8, 170.7, 159.3, 136.9, 129.5 (2C), 128.7 (2C), 128.6, 124.0, 119.2 (2C), 113.6 (2C), 79.7, 72.4, 55.0, 51.1, 33.6, 32.3, 28.5, 24.4, 24.3. MS (ESI) m/z: 400.1 (M+1).

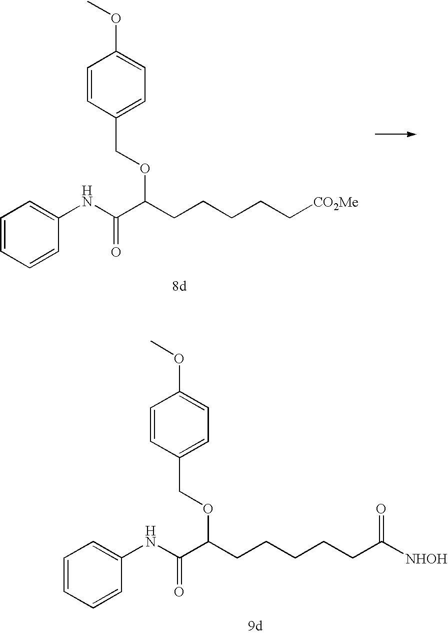

(±)-2-(4-Methoxybenzyloxy)octanedioic acid 8-hydroxyamide 1-phenylamide (9d). Hydroxamic acid 9d was prepared according to the general procedure 7A starting from the corresponding methyl ester 8d in 99% yield. A pale yellow oil: HPLC t_{R}=14.03 min. ¹H-NMR (DMSO, 400 MHz) δ 10.34 (s, 1H), 9.83 (s, 1H), 8.67 (s, 1H), 7.67 (d, J=7.6 Hz, 2H), 7.33-7.29 (m, 1H), 7.07 (t, J=7.4 Hz, 1H), 6.92 (d, J=8.6 Hz, 2H), 4.54 (d, J=11.5 Hz, 1H), 4.34 (d, J=11.5 Hz, 1H), 3.87 (t, J=5.7 Hz, 1H), 3.74 (s, 3H), 1.91 (t, J=7.3 Hz, 2H), 1.70-1.73 (m, 2H), 1.50-1.42 (m, 2H), 1.40-1.80 (m, 4H). ¹H-NMR (CD₃OD, 400 MHz) δ 7.57 (d, J=8.1 Hz, 2H), 7.36-7.32 (m, 4H), 7.14 (t, J=7.4 Hz, 1H), 6.93 (d, J=8.6 Hz, 2H), 4.63 (d, J=11.5 Hz, 1H), 4.48 (d, J=11.6 Hz, 1H), 3.93 (t, J=6.1 Hz, 1H), 3.80 (s, 3H), 2.08 (t, J=7.4, 2H), 1.77 (m, 2H), 1.61 (m, 2H), 1.46 (m, 2H), 1.33 (m, 2H). ¹³C-NMR (CDCl₃, 100 MHz) δ 171.1, 169.1, 158.9, 138.7, 129.9, 129.7 (2C), 128.7 (2C), 123.6, 119.9 (2C), 113.7 (2C), 79.5, 70.9, 55.1, 32.7, 32.3, 28.4, 25.1, 24.7. ¹³C-NMR (CDCl₃, 100 MHz) δ 171.0, 170.9, 159.3, 136.7, 129.6 (2C), 128.7 (2C), 128.6, 124.2, 119.3 (2C), 113.8 (2C), 79.3, 72.4, 55.0, 33.6, 32.1, 27.9, 24.6, 23.8. HRMS (ES+) C₂₂H₂₈N₂O₅ calcd for [MH]⁺ 401.20710. found 401.20598.

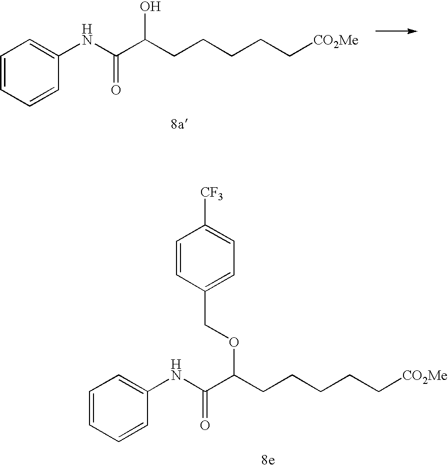

(±)-7-Phenylcarbamoyl-7-(4-trifluoromethylbenzyloxy)heptanoic acid methyl ester (8e). Ether 8e was prepared according to the general procedure (Method 2A) starting from alcohol 8a′ (250 mg, 0.90 mmol), p-trifluoromethylbenzyl bromide (1.08 g, 4.50 mmol) and Ag₂O (313 mg, 1.35 mmol) in anhydrous toluene (5.0 mL) at 50° C. After flash chromatographic purification (gradient 8:2 to 6:4 hexanes/EtOAc) pure O-benzyl ether 8e (146 mg) was obtained in 37% yield as a pale yellow oil: ¹H-NMR (CDCl₃, 400 MHz) δ 8.30 (s, 1H), 7.68 (d, J=8.1 Hz, 2H), 7.63 (d, J=8.1 Hz, 2H), 7.53 (d, J=7.8 Hz, 2H), 7.35 (t, J=8.3 Hz, 2H), 7.16 (t, J=7.4 Hz, 1H), 4.69 (d, J=7.2 Hz, 2H), 4.00 (t, J=5.1 Hz, 1H), 3.67 (s, 3H), 2.31 (t, J=7.4 Hz, 2H), 1.88 (m, 2H), 1.64 (m, 2H), 1.48 (m, 2H), 1.34 (m, 2H). ¹³C-NMR (CDCl₃, 100 MHz) δ 173.8, 170.1, 140.6, 136.7 (2C), 128.8 (2C), 127.6 (2C), 125.3 (2C), 124.3, 119.3 (3C), 80.5, 71.7, 51.2, 33.5, 32.2, 28.4, 24.3, 24.1. MS (ESI) m/z: 438.1 (M+1).

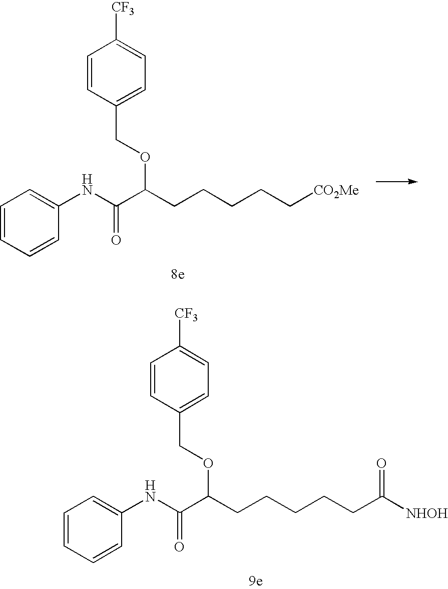

(±)-2-(4-Trifluoromethylbenzyloxy)octanedioic acid 8-hydroxyamide 1-phenylamide (9e). Hydroxamic acid 9e was prepared according to the general procedure 7A starting from the corresponding methyl ester 8e in 99% yield. A pale yellow oil: HPLC t_{R}=6.15 min. ¹H-NMR (CD₃OD, 400 MHz) δ 7.68 (d, J=8.2 Hz, 2H), 7.63 (d, J=8.2 Hz, 2H), 7.58 (d, J=8.0 Hz, 2H), 7.34 (t, J=7.6 Hz, 2H), 7.14 (t, J=7.4 Hz, 1H), 4.79 (d, J=12.4 Hz, 1H), 4.62 (d, J=12.4 Hz, 1H), 4.00 (t, J=5.4 Hz, 1H), 2.09 (t, J=7.4 Hz, 2H), 1.84 (m, 2H), 1.64 (m, 2H), 1.50 (m, 2H), 1.37 (m, 2H). ¹³C-NMR (CDCl₃, 100 MHz) δ 170.6 (2C), 140.5, 136.5 (2C), 128.7 (2C), 127.6 (2C), 125.3 (2C), 124.5, 119.5 (3C), 80.2, 71.7, 32.0 (2C), 28.0, 24.5, 2380. HRMS (ES+) C₂₂H₂₅F₃N₂O₄ calcd for [MH]⁺ 439.18392. found 439.18309.

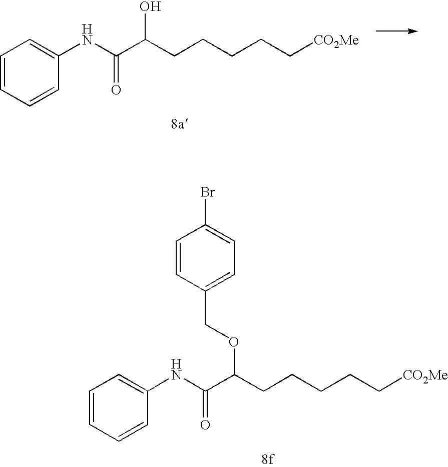

(±)-7-(4-Bromobenzyloxy)-7-phenylcarbamoyl heptanoic acid methyl ester (8f) Ether 8f was prepared according to the general procedure (Method 2A) starting from alcohol 8a′ (250 mg, 0.90 mmol), p-bromobenzyl bromide (1.12 g, 4.50 mmol) and Ag₂O (417 mg, 1.80 mmol) in anhydrous DMF (1.7 mL). After flash chromatographic purification (gradient 9:1 to 75:25 hexanes/EtOAc) pure O-benzyl ether 8f (206 mg) was obtained in 51% yield as a pale yellow oil: ¹H-NMR (CDCl₃, 400 MHz) δ 8.30 (s, 1H), 7.54 (d, J=8.3 Hz, 4H), 7.36 (t, J=8.1 Hz, 2H), 7.27 (d, J=8.4 Hz, 2H), 7.15 (t, J=7.4 Hz, 1H), 4.59 (d, J=5.8 Hz, 2H), 3.98 (t, J=5.0 Hz, 1H), 3.68 (s, 3H), 2.17 (t, J=7.4 Hz, 2H), 1.85 (m, 2H), 1.64 (m, 2H), 1.47 (m, 2H), 1.34 (m, 2H). ¹³C-NMR (CDCl₃, 100 MHz) δ 173.8, 170.2, 136.8, 135.6, 131.5 (2C), 129.3 (2C), 128.7 (2C), 124.2 (2C), 119.2 (2C), 80.2, 71.8, 51.2, 33.6, 33.2, 28.5, 24.4, 24.2. MS (ESI) m/z: 450.2 (M+2).

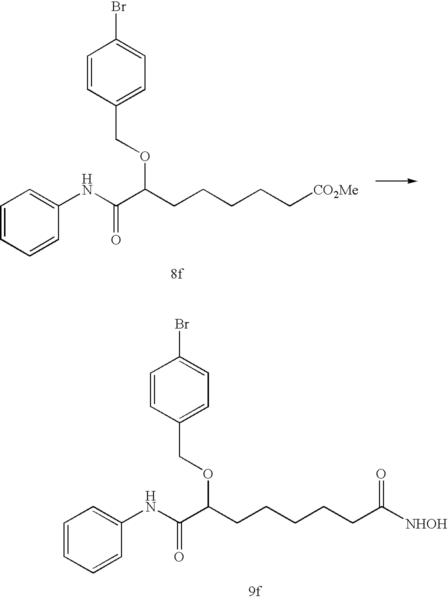

(±)-2-(4-Bromobenzyloxy)octanedioic acid 8-hydroxyamide 1-phenylamide (9t). Hydroxamic acid 9f was prepared according to the general procedure 7A starting from the corresponding methyl ester 8f in 99% yield. A pale yellow oil: HPLC t_{R}=6.06 min. ¹H-NMR (CD₃OD, 400 MHz) δ 7.57 (d, J=8.3 Hz, 2H), 7.53 (d, J=8.3 Hz, 2H), 7.32-7.36 (m, 4H), 7.14 (t, J=7.4 Hz, 1H), 4.67 (d, J=12.0 Hz, 1H), 4.50 (d, J=12.0 Hz, 1H), 3.95 (t, J=5.5 Hz, 1H), 2.09 (t=7.4 Hz, 2H), 1.81 (m, 2H), 1.63 (m, 2H), 1.47 (m, 2H), 1.34 (m, 2H). ¹³C-NMR (CDCl₃, 100 MHz) δ 170.8 (2C), 136.6, 135.5, 131.5 (2C), 129.4 (2C), 128.7 (2C), 124.4, 122.0, 119.4 (2C), 79.9, 71.8, 32.0 (2C), 27.9, 24.6, 23.8. HRMS (ES+) C₂₁H₂₅BrN₂O₄ calcd for [MH]⁺ 449.10705. found 449.10799.

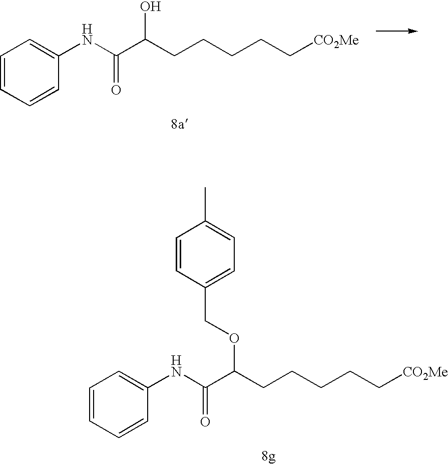

(±)-7-(4-Methylbenzyloxy)-7-phenylcarbamoyl heptanoic acid methyl ester (8 g). Ether 8g was prepared according to the general procedure (Method 2A) starting from alcohol 8a′ (250 mg, 0.90 mmol), p-methylbenzyl bromide (0.91 g, 4.50 mmol) and Ag₂O (313 mg, 1.35 mmol) in anhydrous DMF (1.7 mL). After flash chromatographic purification (gradient 9:1 to 6:4 hexanes/EtOAc) pure O-benzyl ether 8g (86 mg) was obtained in 25% yield as a pale yellow oil: ¹H-NMR (CDCl₃, 400 MHz) δ 8.40 (s, 1H), 7.55 (d, J=7.8 Hz, 2H), 7.35 (t, J=7.7 Hz, 2H), 7.29-7.14 (m, 5H), 4.59 (s, 2H), 3.98 (dd, J=6.9, 2.9 Hz, 1H), 3.68 (s, 3H), 2.39 (s, 3H), 2.31 (t, J=7.5 Hz, 2H), 1.84 (m, 2H), 1.64 (m, 2H), 1.46 (m, 2H), 1.35 (m, 2H). ¹³C-NMR (CDCl₃, 100 MHz) δ 173.8, 170.7, 137.9, 137.0, 133.5, 129.1 (2C), 128.7 (2C), 127.9 (2C), 124.1, 119.2 (2C), 79.9, 72.6, 51.2, 33.6, 32.3, 28.5, 24.4, 24.2, 20.9. MS (ESI) m/z: 384.1 (M+1).

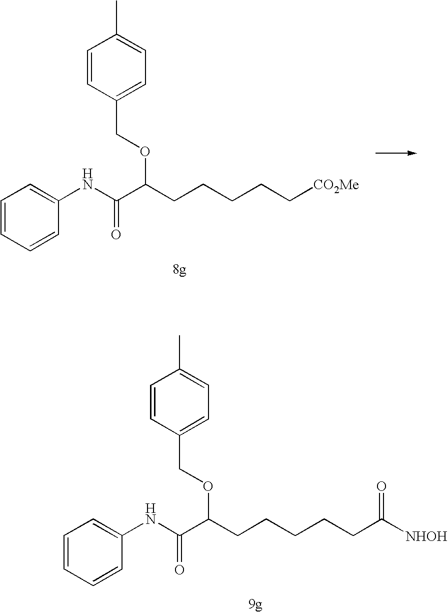

(±)-2-(4-Methylbenzyloxy)octanedioic acid 8-hydroxyamide 1-phenylamide (9g). Hydroxamic acid 9g was prepared according to the general procedure 7A starting from the corresponding methyl ester 8g in 99% yield. A pale yellow oil: HPLC t_{R}=5.99 min. ¹H-NMR (CD₃OD, 400 MHz) δ 7.56 (d, J=8.0 Hz, 2H), 7.34 (t, J=7.6 Hz, 2H), 7.30 (d, J=8.0 Hz, 2H), 7.19 (d, J=7.8 Hz, 2H), 7.13 (t, J=7.4 Hz, 1H), 4.66 (d, J=11.7 Hz, 1H), 4.49 (d, J=11.7 Hz, 1H), 3.93 (t, J=5.8 Hz, 1H), 2.34 (s, 3H), 2.08 (t, J=7.4 Hz, 2H), 1.78 (m, 2H), 1.61 (m, 2H), 1.46 (m, 2H), 1.33 (m, 2H). ¹³C-NMR (CDCl₃, 100 MHz) δ 171.1 (2C), 137.9, 136.7, 133.5, 129.1 (2C), 128.7 (2C), 127.9 (2C), 124.3, 119.4 (2C), 79.7, 72.6, 32.0, 27.9, 24.6, 23.8, 20.9 (2C). HRMS (ES+) C₂₂H₂₈N₂O₄ calcd for [MH]⁺ 385.21218. found 385.21223.

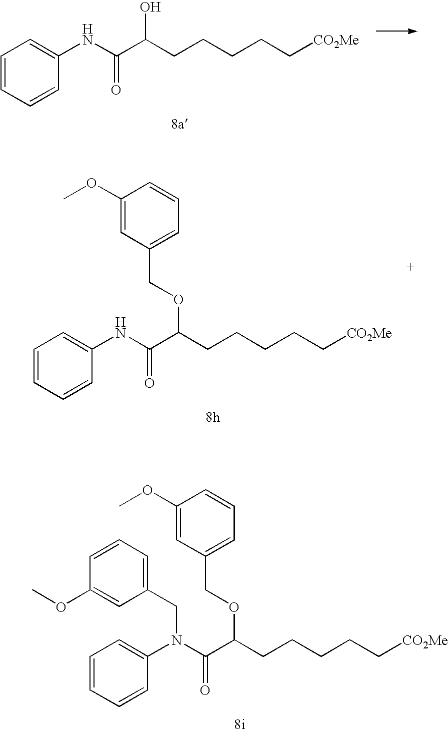

(±)-7-(3-Methoxybenzyloxy)-7-phenylcarbamoyl heptanoic acid methyl ester (8h) and (±)-7-(3-Methoxy-benzyloxy)-7-[(3-methoxybenzyl)phenyl-carbamoyl]heptanoic acid methyl ester (8i). Ethers 8h and 8i were prepared according to the general procedure (Method 2A) starting from alcohol 8a′ (250 mg, 090 mmol), m-methoxybenzyl bromide (0.91 g, 4.50 mmol) and Ag₂O (313 mg, 1.35 mmol) in anhydrous DMF (1.7 mL). After flash chromatographic purification (gradient 9:1 to 6:4 hexanes/EtOAc) pure O-benzyl and N,O-dibenzyl ethers 8h (184 mg) and 8i (112 mg) were isolated in 51% and 24% yield, respectively.

8h, a pale yellow oil: ¹H-NMR (CDCl₃, 400 MHz) δ 8.38 (s, 1H), 7.55 (d, J=7.6 Hz, 2H), 7.35 (t, J=7.8 Hz, 2H), 7.33 (t, J=7.9 Hz, 2H), 7.14 (t, J=7.4 Hz, 1H), 6.92, (m, 2H), 4.61 (s, 2H), 3.99 (dd, J=6.9, 4.6 Hz, 1H), 3.84 (s, 3H), 3.68 (s, 3H), 2.31 (t, J=7.6 Hz, 2H), 1.85 (m, 2H), 1.64 (m, 2H), 1.48 (m, 2H), 1.35 (m, 2H). ¹³C-NMR (CDCl₃, 100 MHz) δ 173.8, 170.5, 159.5, 138.1, 136.9, 129.5, 128.7 (2C), 124.1, 119.9, 119.2 (2C), 113.3 (2C), 80.1, 72.6, 54.9, 51.1, 33.6, 32.3, 28.5, 24.4, 24.2. MS (ESI) m/z: 400.2 (M+1).

8i, a pale yellow oil: ¹H-NMR (CDCl₃, 400 MHz) δ 7.27-7.18 (m, 5H), 6.85-6.76 (m, 8H), 4.93 (d, J=14.1 Hz, 1H), 4.84 (d, J=14.1 Hz, 1H), 4.62 (d, J=12.0 Hz, 1H), 4.33 (d, J=12.0 Hz, 1H), 3.84 (dd, J=8.5, 4.0 Hz, 1H), 3.79 (s, 3H), 3.76 (s, 3H), 3.66 (s, 3H), 2.20 (t, J=7.4 Hz, 2H), 1.74 (m, 2H), 1.58 (m, 2H), 1.51 (m, 2H), 1.34 (m, 2H). ¹³C-NMR (CDCl₃, 100 MHz) 6.173.8, 171.5, 159.3 (2C), 140.7, 139.2, 138.5, 129.1 (2C), 129.0, 128.9, 128.2 (2C), 127.8, 120.9, 119.7 (2C), 113.7, 113.1, 112.6, 74.7, 70.7, 54.8 (2C), 52.9, 51.1, 33.5, 32.1, 28.1, 24.5, 24.3. MS (ESI) m/z: 520.2 (M+1).

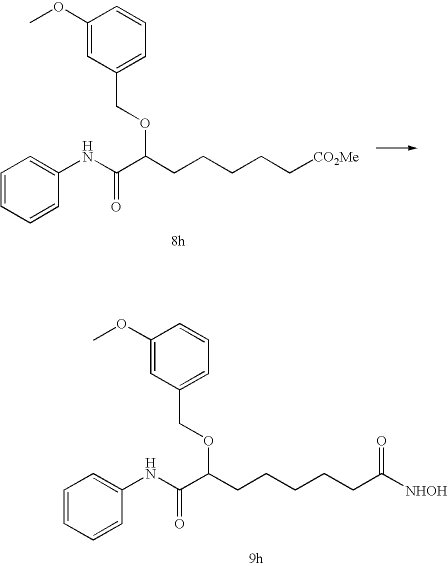

(±)-2-(3-Methoxybenzyloxy)octanedioic acid 8-hydroxyamide 1-phenylamide (9h). Hydroxamic acid 9h was prepared according to the general procedure 7A starting from the corresponding methyl ester 8h in 75% yield. A pale yellow oil: HPLC t_{R}=5.59 min. ¹H-NMR (CD₃OD, 400 MHz) δ 7.57 (d, J=7.3 Hz, 2H), 7.34 (t, J=7.6 Hz, 2H), 7.28 (t, J=7.8 Hz, 1H), 7.14 (t, J=7.4 Hz, 1H), 6.99 (m, 2H), 6.88 (dd, J=8.1, 2.2 Hz, 1H), 4.68 (d, J=12.0 Hz, 1H), 4.52 (d, J=12.0 Hz, 1H), 3.95 (t, J=5.5 Hz, 1H), 3.79 (s, 3H), 2.08 (t, J=7.5 Hz, 2H), 1.80 (m, 2H), 1.62 (m, 2H), 1.47 (m, 2H), 1.34 (m, 2H). ¹³C-NMR (CDCl₃, 100 MHz) δ 170.9 (2C), 159.5, 138.1, 136.7, 129.5, 128.7 (2C), 124.3, 120.0, 119.4 (2C), 113.4, 113.3, 79.8, 72.6, 55.0, 32.0 (2C), 27.9, 24.6, 23.8. HRMS (ES+) C₂₂H₂₈N₂O₅ calcd for [MH]⁺ 401.20710. found 401.20565.

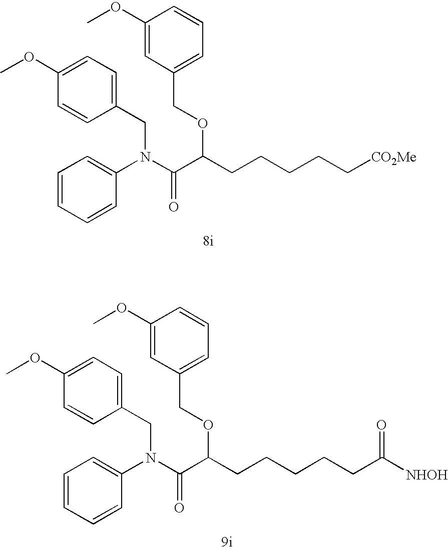

(±)-2-(3-methoxybenzyloxy)octanedioic acid 8-hydroxyamide 1-[(3-methoxybenzyl)phenylamide] (9i). Hydroxamic acid 9i was prepared according to the general procedure 7A starting from the corresponding methyl ester 8i in 86% yield. A pale yellow oil: HPLC t_{R}=6.07 min. ¹H-NMR (CD₃OD, 400 MHz) δ 7.32-7.27 (m, 3H), 7.22 (m, 2H), 6.91 (m, 2H), 6.86-6.82 (m, 4H), 6.76 (m, 2H), 4.98 (d, J=14.3 Hz, 1H), 4.79 (d, J=14.2 Hz, 1H), 4.58 (d, J=12.1 Hz, 1H), 4.33 (d, J=12.2 Hz, 1H), 3.85 (dd, J=8.9, 3.5 Hz, 1H), 3.79 (s, 3H), 3.75 (s, 3H), 1.97 (t, J=7.6 Hz, 2H), 1.67 (m, 2H), 1.57 (m, 2H), 1.44 (m, 2H), 1.33 (m, 2H). ¹³C-NMR (CDCl₃, 100 MHz) δ 171.8, 170.5, 159.2 (2C), 140.4, 139.0, 138.2, 129.2 (2C), 129.1, 129.0, 128.1 (2C), 128.0, 120.9, 119.8 (2C), 113.9, 113.0, 112.9, 74.8, 70.8, 54.9, 54.8, 53.0, 32.0, 31.8, 27.9, 24.5, 24.2. HRMS (ES+) C₃₀H₃₆N₂O₆ calcd for [MH]+ 521.26461. found 521.26331.

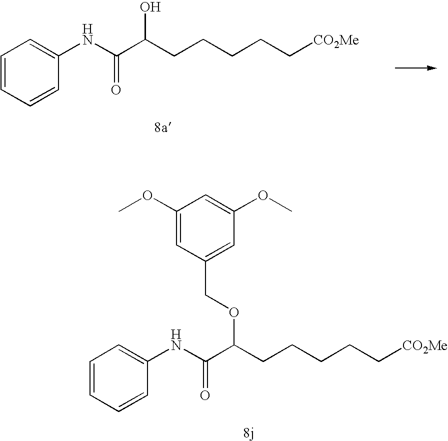

(±)-7-(3,5-Dimethoxy-benzyloxy)-7-phenylcarbamoyl-heptanoic acid methyl ester (8j). Ether 8j was prepared according to the general procedure (Method 2A) starting from alcohol 8a′ (250 mg, 0.90 mmol), 3,5-dimethoxybenzyl bromide (1.04 g, 4.50 mmol) and Ag₂O (417 mg, 1.80 mmol) in anhydrous DMF (1.7 mL). After flash chromatographic purification (gradient 9:1 to 6:4 hexanes/EtOAc) pure O-benzyl ether 8j (128 mg) was obtained in 33% yield as a yellow oil: ¹H-NMR (CDCl₃, 400 MHz) δ 8.38 (s, 1H), 7.55 (d, J=7.6 Hz, 2H), 7.35 (t, J=8.3 Hz, 2H), 7.14 (t, J=7.4 Hz, 1H), 6.53 (d, J=2.3 Hz, 2H), 6.45 (t, J=2.2 Hz, 1H), 4.58 (s, 2H), 3.99 (dd, J=6.8, 4.7 Hz, 1H), 3.81 (s, 6H), 3.67 (s, 3H), 2.31 (t, J=7.4 Hz, 2H), 1.86 (m, 2H), 1.64 (m, 2H), 1.48 (m, 2H), 1.33 (m, 2H). ¹³C-NMR (CDCl₃, 100 MHz) δ 173.8, 170.5, 160.7 (2C), 138.9, 136.9, 128.7 (2C), 124.1, 119.2 (2C), 105.5 (2C), 99.6, 80.1, 72.6, 55.0 (2C), 51.1, 33.6, 32.3, 28.5, 24.4, 24.3. MS (ESI) m/z: 430.1 (M+1), 452.2 (M+Na⁺).

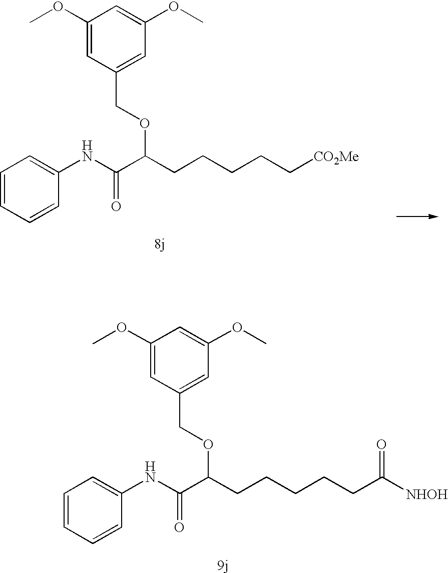

(±)-2-(3,5-Dimethoxybenzyloxy)octanedioic acid 8-hydroxyamide 1-phenylamide (9j). Hydroxamic acid 9j was prepared according to the general procedure 7A starting from the corresponding methyl ester 8j in 68% yield. A pale yellow oil: HPLC t_{R}=5.64 min. ¹H-NMR (CD₃OD, 400 MHz) δ 7.57 (d, J=7.6 Hz, 2H). 7.34 (t, J=7.6 Hz, 2H), 7.14 (t, J=7.4 Hz, 1H), 6.58 (d, J=2.2 Hz, 2H), 6.42 (t, J=2.2 Hz, 1H), 4.65 (d, J=12.1 Hz, 1H), 4.49 (d, J=12.1 Hz, 1H), 3.95 (t, J=5.4 Hz, 1H), 3.77 (s, 6H), 2.09 (t, J=7.4 Hz, 2H), 1.80 (m, 2H), 1.62 (m, 2H), 1.48 (m, 2H), 1.35 (m, 2H). ¹³C-NMR (CDCl₃, 100 MHz) δ 170.9 (2C), 160.7 (2C), 138.9, 136.7 128.7 (2C), 124.3, 119.4 (2C), 105.6 (2C), 99.6, 79.9, 72.6, 55.1 (2C), 32.1 (2C), 27.9, 24.6, 23.9. HRMS (ES+) C₂₃H₃₀N₂O₆ calcd for [MH]⁺ 431.21766. found 431.21790.

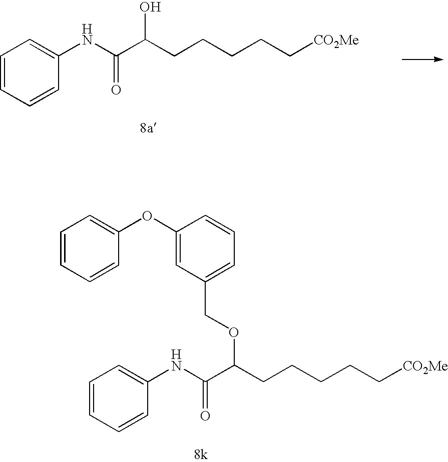

(±)-7-(3-Phenoxybenzyloxy)-7-phenylcarbamoyl heptanoic acid methyl ester (8k). Ether 8k was prepared according to the general procedure (Method 2A) starting from alcohol 8a′ (250 mg, 0.90 mmol), m-phenoxybenzyl bromide (1.18 g, 4.50 mmol) and Ag₂O (313 mg, 1.35 mmol) in anhydrous DMF (1.7 mL). After flash chromatographic purification (gradient 9:1 to 7:3 hexanes/EtOAc) pure O-benzyl ether 8k (100 mg) was obtained in 24% yield as a pale yellow oil: ¹H-NMR (CDCl₃, 400 MHz) δ 8.34 (s, 1H), 7.53 (d, J=7.5 Hz, 2H), 7.39-7.32 (m, 5H), 7.15-7.13 (m, 4H), 7.05-7.03 (m, 3H), 4.61 (s, 2H), 3.98 (dd, J=6.7, 4.7 Hz, 1H), 3.68 (s, 3H), 2.31 (t, J=7.5 Hz, 2H), 1.85 (m, 2H), 1.63 (m, 2H), 1.45 (m, 2H), 1.34 (m, 2H). ¹³C-NMR (CDCl₃, 100 MHz) δ 173.8, 170.4, 157.5, 156.4, 138.6, 136.8, 129.7, 129.5 (2C), 128.7 (2C), 124.1, 123.3, 122.1, 119.2 (2C), 118.8 (2C), 118.1, 117.5, 80.2, 72.2, 51.1, 33.6, 32.2, 28.5, 24.4, 24.2. MS (ESI) m/z: 462.2 (M+1).

3-Phenoxybenzyl bromide. 3-Phenoxybenzyl alcohol (2.09 g, 10.0 mmol) in 18.7 mL of anhydrous CH₂Cl₂ was treated at 0° C. with a solution of PBr₃ (0.35 mL, 3.80 mmol) in CH₂Cl₂ (4.70 mL) and the solution was allowed to reach room temperature during 30 min. The reaction was quenched with saturated aqueous NaHCO₃ and extracted with Et₂O. The organic phase was dried (MgSO₄), concentrated in vacuo and purified by flash chromatography (hexanes/EtOAc (8:2), to afford 1.98 g of bromide as a colorless oil (72% yield). Spectral analysis were consistent to the reported data. (Surman, M. D; Mulvihill, M. J. J. Org. Chem. 2002, 67, 4115-4121).

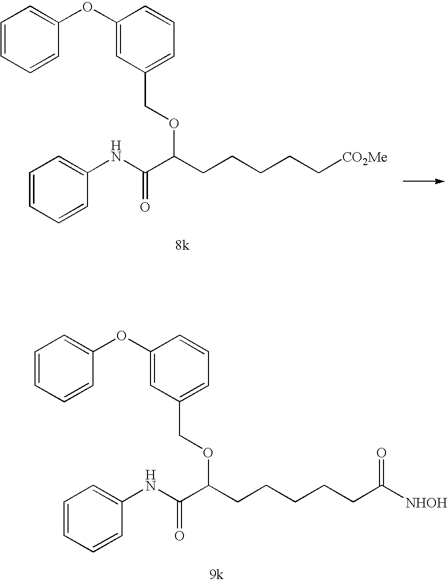

(±)-2-(3-Phenoxybenzyloxy)-octanedioic acid 8-hydroxyamide 1-phenylamide (9k). Hydroxamic acid 9k was prepared according to the general procedure 7A starting from the corresponding methyl ester 8k in 99% yield. A pale yellow oil: HPLC t_{R}=6.52 min. ¹H-NMR (CD₃OD, 400 MHz) δ 7.55 (d, J=7.6 Hz, 2H), 7.38-7.31 (m, 5H), 7.16-7.11 (m, 3H), 7.07 (s, 1H), 6.99-6.93 (m, 3H), 4.68 (d, J=12.2 Hz, 1H), 4.52 (d, J=12.2 Hz, 1H), 3.94 (t, J=5.7 Hz, 1H), 2.08 (t, J=7.4 Hz, 2H), 1.79 (m, 2H), 1.61 (m, 2H), 1.45 (m, 2H), 1.33 (m, 2H). ¹³C-NMR (CDCl₃, 100 MHz) δ 170.7 (2C), 157.4, 156.4, 138.6, 136.6, 129.8, 129.5 (2C), 128.7 (2C), 124.3, 123.3, 122.2, 119.4 (2C), 118.8 (2C), 118.1, 117.6, 79.9, 72.2, 32.0 (2C), 27.9, 24.5, 23.7. HRMS (ES+) C₂₇H₃₀N₂O₅ calcd for [MH]+ 463.22275. found 463.22270.

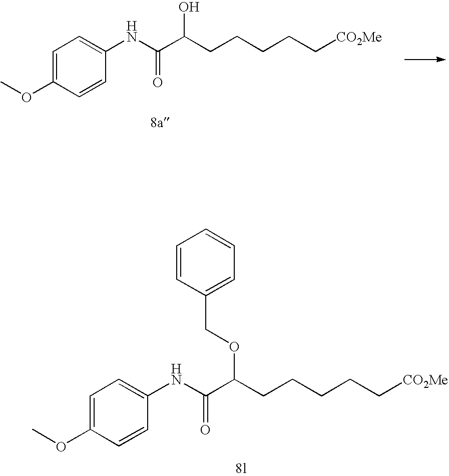

(±)-7-Benzyloxy-7-(4-methoxyphenylcarbamoyl)heptanoic acid methyl ester (8l). Ether 8l was prepared according to the general procedure (Method 2A) starting from alcohol 8a″ (250 mg, 0.81 mmol), benzyl bromide (0.48 mL, 4.04 mmol) and Ag₂O (0.38 g, 1.62 mmol) in anhydrous DMF (1.50 mL). After filtration and flash chromatographic purification (gradient 9:1 to 7:3 hexanes/EtOAc) pure 0-benzyl ether 8l was obtained in 41% yield as a pale yellow oil: ¹H-NMR (CDCl₃, 400 MHz) δ 8.29 (s, 1H), 7.46 (d, J=9.0 Hz, 2H), 7.42-7.37 (m, 5H), 6.89 (d, J=9.0 Hz, 2H), 4.64 (s, 2H), 3.99 (dd, J=6.9, 4.5 Hz, 1H), 3.82 (s, 3H), 3.68 (s, 3H), 2.31 (t, J=7.5 Hz, 2H), 1.83 (m, 2H), 1.64 (m, 2H), 1.48 (m, 2H), 1.35 (m, 2H). ¹³C-NMR (CDCl₃, 100 MHz) δ 173.8, 170.2, 156.1, 136.6, 130.1, 128.4 (2C), 128.0, 127.7 (2C), 120.9 (2C), 113.8 (2C), 80.0, 72.7, 55.1, 51.1, 33.6, 32.3, 28.5, 24.4, 24.2. MS (ESI) m/z: 400.1 (M+1).

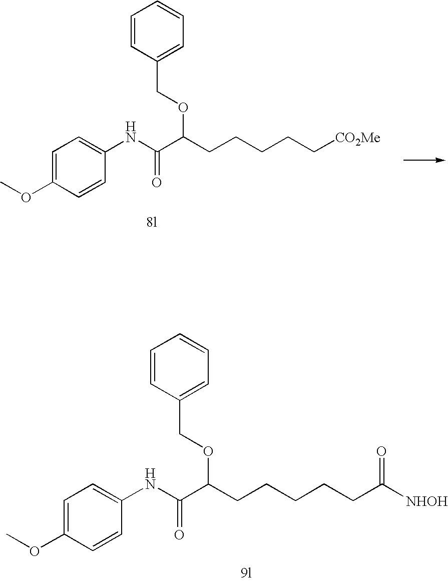

(±)-2-Benzyloxyoctanedioic acid 8-hydroxyamide 1-[(4-methoxyphenyl)-amide] (9l). Hydroxamic acid 9l was prepared according to the general procedure 7A starting from the corresponding methyl ester 8l in 80% yield. A pale yellow oil (80% yield): HPLC t_{R}=5.50 min. ¹H-NMR (CDl₃OD, 400 MHz) δ 7.46 (d, J=9.0 Hz, 2H), 7.44-7.30 (m, 5H), 6.91 (d, J=9.0 Hz, 2H), 4.71 (d, J=11.8 Hz, 1H), 4.52 (d, J=11.8 Hz, 1H), 3.94 (t, J=5.6 Hz, 1H), 3.80 (s, 3H), 2.08 (t, J=7.4 Hz, 2H), 1.79 (m, 2H), 1.62 (m 2H), 1.45 (m, 2H), 1.34 (m, 2H). ¹³C-NMR (CDCl₃, 100 MHz) δ 170.8, 170.6, 156.2, 136.6, 129.8, 128.4 (2C), 128.0, 127.8 (2C), 121.2 (2C), 113.8 (2C), 79.7, 72.7, 55.1, 32.0 (2C), 27.8, 24.5, 23.7. HRMS (ES+) C₂₂H₂₈N₂O₅ calcd for [MH]⁺ 401.20710. found 401.20668.

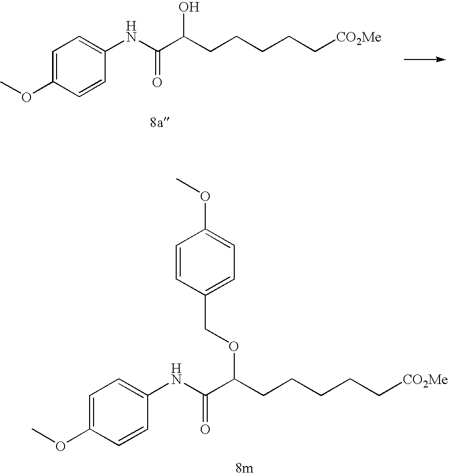

(±)-7-(4-Methoxybenzyloxy)-7-(4-methoxyphenylcarbamoyl)heptanoic acid methyl ester (8m). Ether 8m was prepared according to the general procedure (Method 2B) starting from 250 mg of alcohol 8a″ (0.81 mmol) in 1.2 mL of Et₂O in the presence of catalytic BF₃.Et₂O (1 μL, 8×10⁻³ mmol). After purification by flash chromatography (gradient 8:2 to 4:6 hexanes/EtOAc) pure p-methoxybenzyl ether 8m was recovered in 35% yield as a pale yellow oil: ¹H-NMR (CDCl₃, 400 MHz) δ 8.28 (s, 1H), 7.46 (d, J=8.9 Hz, 2H), 7.31 (d, J=8.5 Hz, 2H), 6.91 (dd, J=19.1, 8.3 Hz, 4H), 4.56 (s, 2H), 3.96 (dd, J=6.9, 4.4 Hz, 1H), 3.84 (s, 3H), 3.82 (s, 3H), 3.68 (s, 3H), 2.30 (t, J=7.5 Hz, 2H), 1.82 (m, 2H), 1.63 (m, 2H), 1.45 (m, 2H), 1.32 (m, 2H). ¹³C-NMR (CDCl₃, 100 MHz) δ 173.8, 170.4, 159.3, 156.1, 130.1, 129.5 (2C), 128.7, 120.9 (2C), 113.8 (2C), 113.7 (2C), 79.7, 72.4, 55.1, 55.0, 51.1, 33.6, 32.4, 28.5 24.4, 24.3. MS (ESI) m/z: 430.2 (M+1).

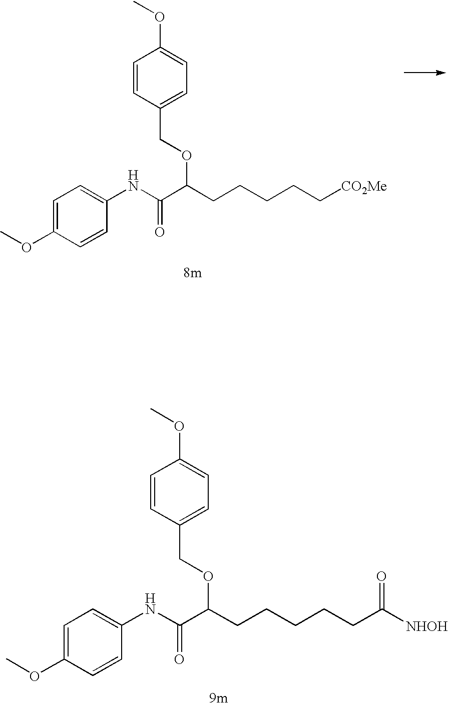

(±)-2-(4-Methoxybenzyloxy)octanedioic acid 8-hydroxyamide 1-[(4-methoxyphenyl)amide] (9m). Hydroxamic acid 9m was prepared according to the general procedure 7A starting from the corresponding methyl ester 8m in 99% yield. A pale yellow oil: HPLC t_{R}=5.59 min. ¹H-NMR (CD₃OD, 400 MHz) δ 7.45 (d, J=9.1 Hz, 2H), 7.34 (d, J=8.6 Hz, 2H), 6.94-6.89 (m, 4H), 4.63 (d, J=11.2 Hz, 1H), 4.46 (d, J=11.5 Hz, 1H), 3.91 (t, J=6.1 Hz, 1H), 3.80 (s, 6H), 2.08 (t, J=7.4 Hz, 2H), 1.76 (m, 2H), 1.60 (m, 2H), 1.45 (m, 2H), 1.33 (m, 2H). ¹³C-NMR (CDCl₃, 100 MHz) δ 170.8 (2C), 159.3, 156.2, 129.8, 129.5 (2C), 128.7, 121.1 (2C), 113.8 (4C), 79.3, 72.4, 55.1, 55.0, 32.1 (2C), 27.9, 24.6, 32.8. HRMS (ES+) C₂₃H₃₀N₂O₆ calcd for [MH]⁺ 431.21766. found 431.21643.

### Example 3

### Preparation of racemic ω-alkoxy derivatives having 9 carbon long chain

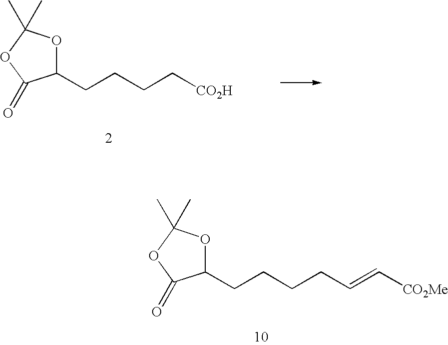

(±)-7-(2,2-Dimethyl-5-oxo-[1,3]dioxolan-4-yl)hept-2-enoic acid methyl ester (10). Under an argon atmosphere, BH₃.DMS (8.3 mL, 2.0 M solution in THF) was added dropwise to a solution of carboxylic acid 2 (1.80 g, 8.3 mmol) in THF (155 mL) cooled at 0° C. The resulting solution was stirred at less than 10° C. for 2 h, then was quenched by the slow addition of MeOH at 0° C. and concentrated under vacuum. The residue was taken up in CH₂Cl₂/H₂O and the aqueous layer was extracted with further portions of CH₂Cl₂. All the combined organic extracts were dried (MgSO₄), filtered and concentrated in vacuo. Purification by flash chromatography (6:4 hexanes/EtOAc) afforded alcohol intermediate (1.46 g) in 87% yield as a colorless oil: ¹H-NMR (CDCl₃, 300 MHz) δ 4.41, (dd, J=7.0, 4.4 Hz, 1H), 3.66 (t, J=6.4 Hz, 2H), 1.95-1.70 (m, 3H), 1.62 (s, 3H), 1.64-1.40 (m, 9H). ¹³C-NMR (CDCl₃, 75 MHz) δ 173.8, 110.9, 74.5, 63.2, 32.9, 31.9, 27.6, 26.2, 25.8, 25.1. MS (ESI) m/z: 203.1 (M+1).

To a solution of oxalyl chloride (1.75 mL, 20.1 mmol) in CH₂Cl₂ (10 mL) at −78° C. under argon a solution of dimethylsulfoxide (DMSO) (1.9 mL, 26.8 mmol) in CH₂Cl₂ (20 mL) was added dropwise. After 10 min a solution of the above alcohol intermediate (1.35 g, 6.7 mmol) in CH₂Cl₂ (38 mL) was added. The reaction mixture was stirred at −78° C. for 30 min, then Et₃N (9.34 mL, 67.0 mL) was added and stirring was continued for 30 min. After warming over 2 h to room temperature toluene (30 mL) was added, and the mixture was filtered and concentrated under vacuum. The crude residue was subjected to flash chromatographic purification (7:3 hexanes/EtOAc). Pure aldehyde intermediate (1.32 g, 98%) was recovered as a colorless oil: ¹H-NMR (CDCl₃, 300 MHz) δ 9.80 (t, J=1.6 Hz, 1H), 4.42 (dd, J=7.0, 4.4 Hz, 1H), 2.50 (dt, J=7.1, 1.6 Hz, 2H), 1.92 (m, 1H), 1.83-1.67 (m, 3H), 1.63 (s, 3H), 1.57-1.45 (m, 5H). ¹³C-NMR (CDCl₃, 75 MHz) δ 202.6, 173.4, 110.9, 74.3, 44.0, 31.6, 27.6, 26.1, 24.9, 22.0. MS (ESI) m/z: 201.1 (M+1).

To a stirring solution of this aldehyde intermediate (1.25 g, 6.2 mmol) in CH₂Cl₂ (60 mL) Ph₃P═CHCH₂^{t}Bu (3.11 g, 9.3 mmol) was added while stirring at room temperature. The resulting solution was stirred for 4 h and then evaporated to dryness. The crude was purified by flash chromatography (8:2 hexanes/EtOAc) which afforded unsaturated methyl ester 10 (1.54 g) in 96% yield as a sole trans isomer. A colorless oil: ¹H-NMR (CDCl₃, 300 MHz) δ 6.97 (dt, J=15.6, 7.0 Hz, 1H), 5.85 (dt, J=15.6, 1.6 Hz, 1H), 4.40 (dd, J=7.2, 4.3 Hz, 1H), 3.75 (s, 3H), 2.50 (m, 2H), 1.90 (m, 1H), 1.76 (m, 1H), 1.62 (s, 3H), 1.56-1.46 (m, 7H). ¹³C-NMR (CDCl₃, 100 MHz) δ 173.6, 167.5, 149.4, 121.6, 110.9, 74.4, 51.9, 32.4, 31.7, 28.0, 27.6, 26.2, 24.9. MS (ESI) m/z: 257.1 (M+1), 279.1 (M+Na⁺).

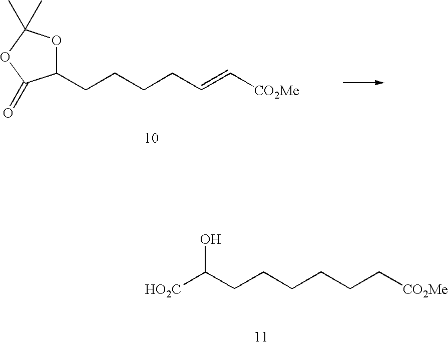

### (±)-2-Hydroxynonanedioic acid 9-methyl ester (11)

A solution of this unsaturated ester intermediate (1.50 g, 5.9 mmol) in 59 mL of absolute MeOH was cooled to 0° C. and treated with NiCl₂.6H₂O (352 mg, 1.48 mmol). The resulting mixture was stirred at the same temperature for 15 min before the addition of NaBH₄ (114 mg, 2.95 mg). After 30 min an additional portion of NaBH₄ 856 mg, 1.48 mmol) was added and the reaction was allowed to stir for additional 10 min. The reaction was quenched with NH₄Cl (aq, sat.) and extracted with CH₂Cl₂. The combined extracts were dried (MgSO₄) and concentrated under reduced pressure to afford saturated intermediate (1.50 g, 98%) as a colorless oil: ¹H-NMR (CDCl₃, 300 MHz) δ 4.38 (dd, J=7.0, 4.5 Hz, 1H), 3.66 (s, 3H), 2.30 (t, J=5.6 Hz, 2H), 1.86 (m, 1H), 1.70 (m, 1H), 1.65-1.59 (m, 2H), 1.59 (s, 3H), 1.53 (s, 3H), 1.49-1.32 (m, 6H). ¹³C-NMR (CDCl₃, 75 MHz) δ 173.8, 173.0, 110.0, 73.7, 51.1, 33.6, 31.0, 28.5, 28.4, 26.8, 25.4, 24.4, 24.3. MS (ESI) m/z: 259.1 (M+1), 281.1 (M+Na⁺).

This fully protected, saturated intermediate (1.40 g, 5.4 mmol) was suspended in 10 mL of 70% aqueous acetic acid and stirred for 2 h at 60° C. After cooling at room temperature H₂O was added (28 mL), and the mixture was extracted with EtOAc. All the combined organic extracts were dried (MgSO₄), filtered, and concentrated under vacuum to afford crude 11 as an oily residue (1.11 g, 94%) which was used without further purification: ¹H-NMR (CDCl₃, 300 MHz) δ 7.20 (b, 1H), 4.29 (dd, J=7.4, 4.2 Hz, 1H), 3.69 (s, 3H), 2.34 (t, J=7.4 Hz, 2H), 2.15 (s, 1H), 1.87 (m, 1H), 1.78-1.58 (m, 3H), 1.52-1.31 (m, 6H). ¹³C-NMR (CDCl₃, 75 MHz) δ 179.9, 175.0, 70.6, 52.1, 34.5, 34.4, 29.3, 29.2, 25.2, 24.9. MS (ESI) m/z: 219.1 (M+1).

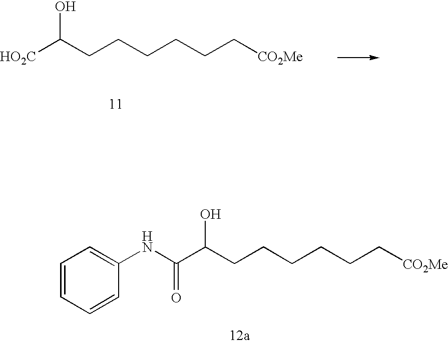

(±)-8-Hydroxy-8-phenylcarbamoyl octanoic acid methyl ester (12a). Anilide 12a was prepared according to the general procedure (Method 1A), starting from 2-hydroxy acid 11 (1.05 g, 4.81 mmol), N-sulfinylaniline (0.94 g, 6.74 mmol) and 1,2,4-triazole (0.47 g, 6.74 mmol). After flash chromatographic purification (gradient 7:3 to 1:1 hexanes/EtOAc) pure 12a (1.35 g) was recovered in 96% yield as a yellow solid: ¹H-NMR (CDCl₃, 400 MHz) δ 8.53 (s, 1H), 7.58 (d, J=8.4 Hz, 2H), 7.35 (t, J=8.2 Hz, 2H), 7.14 (t, J=7.4 Hz, 1H), 4.24 (dd, J=7.8, 4.0 Hz, 1H), 3.69 (s, 3H), 3.20 (b, 1H), 2.34 (t, J=7.4 Hz, 2H), 1.92 (m, 1H), 1.74 (m, 1H), 1.63 (m, 2H), 1.48 (m, 2H), 1.31-1.38 (m, 4H). ¹³C-NMR (CDCl₃, 100 MHz) δ 174.1, 171.6, 136.9, 128.7 (2C), 124.1, 119.4 (2C), 72.1, 51.2, 34.2, 33.6, 28.5 (2C), 24.3 (2C). MS (ESI) m/z: 294.2 (M+1), 316.2 (M+Na⁺).

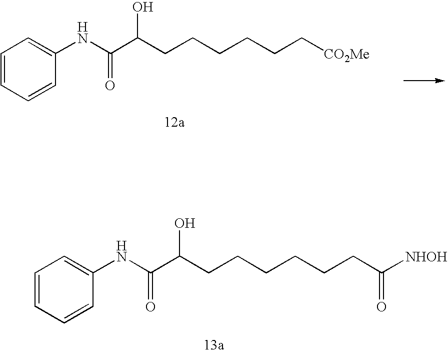

(±)-2-Hydroxynonanedioic acid 9-hydroxyamide 1-phenylamide (13a). Hydroxamic acid 13a was prepared according to the general procedure 7A starting from the corresponding methyl ester 12a in 74% yield. A white solid: HPLC t_{R}=6.75 min. ¹H-NMR (CD₃OD, 400 MHz) δ 7.60 (d, J=7.5 Hz, 2H), 7.34 (t, J=7.5 Hz, 2H), 7.13 (t, J=7.4 Hz, 1H), 4.14 (dd, J=7.8, 4.0 Hz, 1H), 2.10 (t, J=7.3 Hz, 2H), 1.85 (m, 1H), 1.75-1.59 (m, 3H), 1.50 (m, 2H), 1.40-1.33 (m, 4H). ¹³C-NMR (CD₃OD, 100 MHz) δ 173.9, 171.2, 137.2, 128.1 (2C), 123.8, 119.8 (2C), 71.3, 33.9, 32.0, 28.4, 28.2, 24.9, 24.2. HRMS (ES+) C₁₅H₂₂N₂O₄ calcd for [MH]+ 295.16523. found 295.16543.

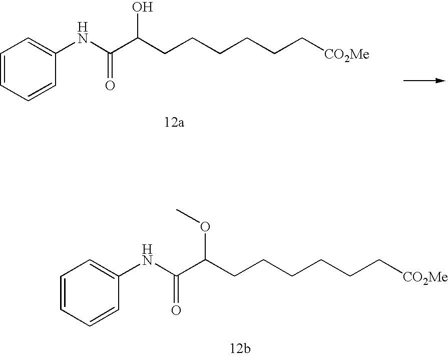

(±)-8-Methoxy-8-phenylcarbamoyloctanoic acid methyl ester (12b). Ether 12b was prepared according to the general procedure (Method 2A) starting from alcohol 12a (250 mg, 0.85 mmol), methyl iodide (1.33 mL, 21.25 mmol) and Ag₂O (0.24 g, 1.02 mmol) in anhydrous MeCN (1.20 mL) under reflux temperature. After purification by flash chromatography (gradient 8:2 to 6:4 hexanes/EtOAc) O-methyl ether 12b (194 mg) was recovered in 74% yield as a colorless oil: ¹H-NMR (CDCl₃, 400 MHz) δ 8.32 (s, 1H), 7.60 (dd, J=8.5, 1.1 Hz, 2H), 7.35 (t, J=7.6 Hz, 2H), 7.13 (t, J=7.4 Hz, 1H), 3.75 (dd, J=6.7, 4.5 Hz, 1H), 3.67 (s, 3H), 3.49 (s, 3H), 2.31 (t, J=7.4 Hz, 2H), 1.81 (m, 2H), 1.62 (m, 2H), 1.42 (m, 2H), 1.37-1.31 (m, 4H). ¹³C-NMR (CDCl₃, 100 MHz) δ 173.9, 170.4, 136.9, 128.7 (2C), 124.0, 119.2 (2C), 82.3, 58.1, 51.1, 33.7, 31.9, 28.7, 28.6, 24.5, 24.1. MS (ESI) m/z: 308.2 (M+1), 330.2 (M+Na⁺).

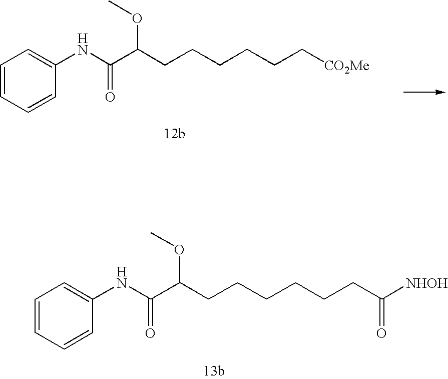

(±)-2-Methoxynonanedioic acid 9-hydroxyamide 1-phenylamide (13b). Hydroxamic acid 13b was prepared according to the general procedure 7A starting from the corresponding methyl ester 12b in 85% yield. A colorless oil: HPLC t_{R}=4.76 min. ¹H-NMR (CD₃OD, 400 MHz) δ 7.61 (d, J=7.7 Hz, 2H), 7.34 (t, J=7.6 Hz, 2H), 7.14 (t, J=7.4 Hz, 1H), 3.77 (t, J=5.9 Hz, 1H), 3.44 (s, 3H), 2.10 (t, J=7.4 Hz, 2H), 1.79 (m, 2H), 1.63 (m, 2H), 1.46 (m, 2H), 1.40-1.32 (m, 4H). ¹³C-NMR (CDCl₃, 100 MHz) δ 171.2, 170.9, 136.8, 128.7 (2C), 124.2, 119.4 (2C), 82.1, 58.2, 32.3, 31.7, 28.2, 28.1, 24.7, 23.8. HRMS (ES+) C₁₆H₂₄N₂O₄ calcd for [MH]+ 309.18088. found 309.18072.

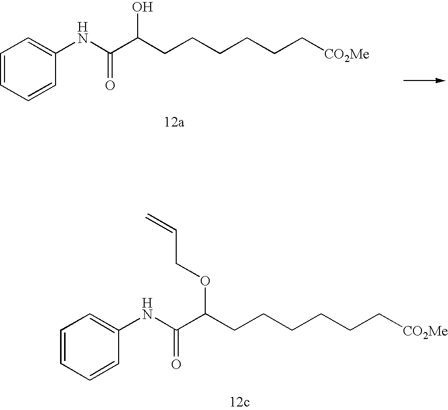

(±)-8-Allyloxy-8-phenylcarbamoyloctanoic acid methyl ester (12c). Ether 12c was prepared according to the general procedure (Method 2A) starting from alcohol 12a (250 mg, 0.85 mmol), allyl iodide (1.94 mL, 21.30 mmol) and Ag₂O (0.24 g, 1.02 mmol) in anhydrous MeCN (1.40 mL) at 45° C. After purification by flash chromatography (gradient 9:1 to 7:3 hexanes/EtOAc) O-allyl ether 12c (193 mg) was isolated in 68% yield as a pale yellow oil: ¹H-NMR (CDCl₃, 400 MHz) δ 8.38 (s, 1H), 7.58 (dd, J=8.4, 0.8 Hz, 2H), 7.35 (t, J=8.4 Hz, 2H), 7.14 (t, J=7.4 Hz, 1H), 5.97 (ddt, J=17.2, 10.4, 5.7 Hz, 1H), 5.38 (dd, J=17.2, 1.5 Hz, 1H), 5.29 (dd, J=10.4, 1.3 Hz, 1H), 4.12 (d, J=5.7 Hz, 2H), 3.91 (dd, J=7.0, 4.4 Hz, 1H), 3.67 (s, 3H), 2.31 (t, J=7.4 Hz, 2H), 1.83 (m, 2H), 1.63 (m, 2H), 1.45 (m, 2H), 1.37-1.31 (m, 4H). ¹³C-NMR (CDCl₃, 100 MHz) δ 173.9, 170.6, 136.9, 133.3, 128.7 (2C), 124.0, 119.2 (2C), 117.9, 79.9, 71.4, 51.1, 33.7, 32.4, 28.7 28.6, 24.5, 24.4. MS (ESI) m/z: 334.1 (M+1).

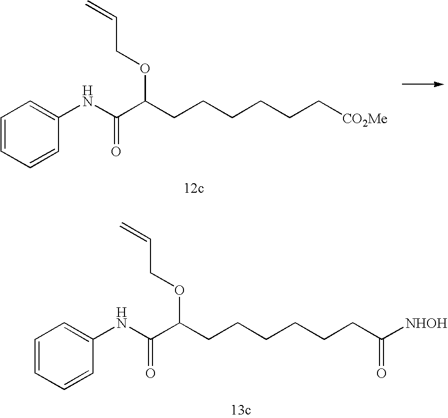

(±)-2-Allyloxynonanedioic acid 9-hydroxyamide 1-phenylamide (13c). Hydroxamic acid 13c was prepared according to the general procedure 7A starting from the corresponding methyl ester 12c in 85% yield. A pale yellow oil: HPLC t_{R}=5.28 min. ¹H-NMR (CD₃OD, 400 MHz) δ 7.60 (d, J=7.6 Hz, 2H), 7.34 (t, J=7.5 Hz, 2H), 7.14 (t, J=7.4 Hz, 1H), 6.00 (ddt, J=17.1, 10.4, 5.7 Hz, 1H), 5.35 (d, J=17.2 Hz, 1H), 5.24 (d, J=10.4 Hz, 1H), 4.18 (dd, J=12.8, 5.5 Hz, 1H), 4.03 (dd, J=12.7, 6.0 Hz, 1H), 3.93 (t, J=6.1 Hz, 1H), 2.10 (t, J=7.3 Hz, 2H), 1.80 (m, 2H), 1.63 (m, 2H), 1.49 (m, 2H), 1.42-1.32 (m, 4H). ¹³C-NMR (CDCl₃, 100 MHz) δ 171.1, 171.0, 136.7, 133.2, 128.7 (2C), 124.2, 119.4 (2C), 188.1, 79.7, 71.5, 32.3, 32.1, 28.2, 28.0, 24.7, 24.0. HRMS (ES+) C₁₈H₂₆N₂O₄ calcd for [MH]+ 335.19653. found 335.19653.

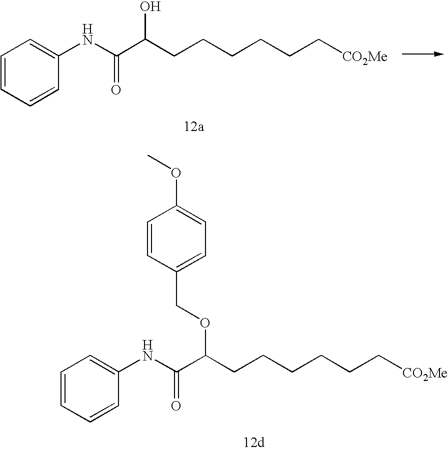

(±)-8-(4-Methoxy-benzyloxy)-8-phenylcarbamoyloctanoic acid methyl ester (12d). Ether 12d was prepared according to the general procedure (Method 2A) starting from alcohol 12a (250 mg, 0.85 mmol) p-methoxybenzyl bromide (a freshly prepared 2 M solution in toluene, 10.63 mL) and Ag₂O (0.24 g, 1.02 mmol). After purification by flash chromatography (gradient 9:1 to 7:3 hexanes/EtOAc) intermediate p-methoxybenzyl ether 12d (112 mg) was recovered in 31% yield as a yellow oil: ¹H-NMR (CDCl₃, 400 MHz) δ 8.39 (s, 1H), 7.55 (d, J=7.6 Hz, 2H), 7.37-7.28 (m, 4H), 7.14 (t, J=7.4 Hz, 1H), 6.90 (d, J=8.0 Hz, 2H), 4.57 (s, 2H), 3.97 (dd, J=7.1, 4.3 Hz, 1H), 3.83 (s, 3H), 3.68 (s, 3H), 2.31 (t, J=7.5 Hz, 2H), 1.83 (m, 2H), 1.63 (m, 2H), 1.45 (m, 2H), 1.33-1.27 (m, 4H). ¹³C-NMR (CDCl₃, 100 MHz) δ 173.9, 170.8, 159.3, 136.8, 129.5 (2C), 128.7 (2C), 128.3, 124.0, 119.2 (2C), 113.7 (2C), 79.8, 72.4, 55.0, 51.1, 33.7, 32.5, 28.6 (2C), 24.5, 24.4. MS (ESI) m/z: 414.1 (M+1).

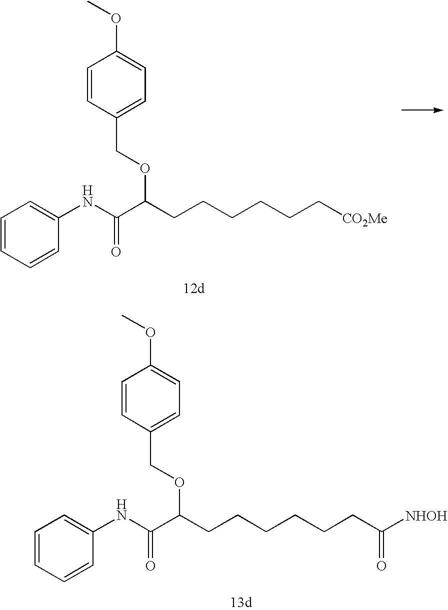

(±)-2-(4-Methoxybenzyloxy)nonanedioic acid 9-hydroxyamide 1-phenylamide (13d). Hydroxamic acid 13d was prepared according to the general procedure 7A starting from the corresponding methyl ester 12d in 75% yield. A yellow oil: HPLC t_{R}=6.03 min. ¹H-NMR (CD₃OD, 400 MHz) δ 7.57 (d, J=7.6 Hz, 2H), 7.36-7.32 (m, 4H), 7.14 (t, J=7.4 Hz, 1H), 6.92 (d, J=8.6 Hz, 2H), 4.63 (d, J=11.6 Hz, 1H), 4.46 (d, J=11.6 Hz, 1H), 3.93 (t, J=6.0 Hz, 1H), 3.80 (s, 3H), 2.08 (t, J=7.3 Hz, 2H), 1.77 (m, 2H), 1.60 (m, 2H), 1.44 (m, 2H), 1.34-1.29 (m, 4H). ¹³C-NMR (CDCl₃, 100 MHz) δ 171.0, 170.9, 159.3, 136.8, 129.5 (2C), 128.7 (2C), 128.6, 124.2, 119.3 (2C), 113.8 (2C), 79.6, 72.5, 55.0, 32.2, 32.1, 28.0, 27.8, 24.6, 24.0. HRMS (ES+) C₂₃H₃₀N₂O₅ calcd for [MH]⁺ 415.22275. found 415.22230.

### Example 4

### Preparation of Enantiopure 7 Carbon Long Chain Linear ω-Alkoxy Derivatives

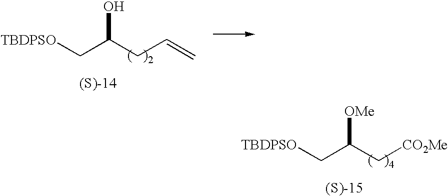

7-(tert-Butyldiphenylsilanyloxy)-6-(S)-methoxy heptanoic acid methyl ester [(S)-15]. To a stirring solution of olefin (S)-14 (Dixon, D. J.; Steven V. Ley, S. V.; Reynolds, D. J. Chem. Eur. J. Org. Chem. 2002, 8, 1621-1636) (1.00 g, 2.82 mmol) and methyl iodide (0.26 mL, 4.23 mmol) in toluene (28.0 mL) at −78° C. under an argon atmosphere, KHMDS (0.5 M solution in toluene, 6.77 mL) was added dropwise. The reaction was warmed to ambient temperature in a period of 30 min, and then allowed to stir until complete conversion of the starting material. After 1 h the reaction was quenched with NH₄Cl (aq, sat.) and extracted with CH₂Cl₂. The organic phase was dried (MgSO₄) and concentrated in vacuo to furnish a crude residue which was purified by flash chromatography (95:15 hexanes/EtOAc). O-Methyl ether intermediate was recovered as a colorless oil in 95% yield (0.99 g): [α]²⁰_{D} −6.3 (c 2.9, CHCl₃). ¹H-NMR (CDCl₃, 400 MHz) δ 7.74-7.70 (m, 4H), 7.46-7.40 (m, 6H), 5.84 (ddt, J=16.9, 10.3, 6.7 Hz, 1H), 5.04 (dd, J=17.1, 1.9 Hz, 1H), 4.99 (dd, J=10.1, 2.0 Hz, 1H), 3.71 (dd, J=10.7, 5.5 Hz, 1H), 3.63 (dd, J=10.7, 4.8 Hz, 1H), 3.40 (s, 3H), 3.29 (m, 1H), 2.22-2.06 (m, 2H), 1.71-1.54 (m, 2H), 1.08 (s, 9H). ¹³C-NMR (CDCl₃, 100 MHz) δ 138.3, 135.3 (4C), 133.2 (2C), 129.3 (2C), 127.3 (4C), 114.2, 80.8, 65.1, 57.7, 30.4, 29.2, 26.5 (3C), 18.9. MS (ESI) m/z: 391.3 (M+Na⁺).

This intermediate was subjected to cross-metathesis reaction according to the general procedure 4A, coupling it with methyl acrylate (2.89 mL, 32.16 mmol) in the presence of Grubbs' catalyst 2^{nd} generation (68 mg, 0.08 mmol) in anhydrous CH₂Cl₂ (5.4 mL). After purification by flash chromatography (9:1 hexanes/EtOAc) the unsaturated ester intermediate was recovered as a colorless oil in 93% yield (1.15 g): [α]²⁰_{D} − 4.3 (c 3.4, CHCl₃). ¹H-NMR (CDCl₃, 400 MHz) δ 7.71-7.69 (m, 4H), 7.46-7.38 (m, 6H), 7.00 (dt, J=17.6, 3.7 Hz, 1H), 5.85 (dt, J=15.6, 1.5 Hz, 1H), 3.76 (s, 3H), 3.70 (dd, J=10.5, 5.0 Hz, 1H), 3.61 (dd, J=10.7, 5.2 Hz, 1H), 3.66 (s, 3H), 3.25 (m, 1H), 2.29 (m, 2H), 1.66 (m, 2H), 1.08 (m, 9H). ¹³C-NMR (CDCl₃, 100 MHz) δ 165.8, 149.0, 135.3 (4C), 133.1 (2C), 129.4 (2C), 127.3 (4C), 120.7, 80.4, 64.7, 57.6, 51.1, 29.5, 27.7, 26.5 (3C), 18.8. MS (ESI) m/z: 444.3 (M+18), 449.2 (M+Na⁺).

This olefin intermediate was hydrogenated according to the general procedure (Method 6A). After flash chromatography (8:2 hexanes/EtOAc), saturated ester (S)-15 (1.10 g) was obtained in 96% yield as a colorless oil: [α]²⁰_{D} −9.1 (c 1.8, CHCl₃). ¹H-NMR (CDCl₃, 400 MHz) δ 7.71-7.69 (m, 4H), 7.45-7.39 (m, 6H), 3.71-3.68 (m, 1H), 3.69 (s, 3H), 3.61 (dd, J=10.7, 4.9 Hz, 1H), 3.39 (s, 3H), 3.24 (dd, J=7.4, 5.0 Hz, 1H), 2.32 (t, J=7.6 Hz, 2H), 1.69-1.23 (m, 6H), 1.08 (s, 9H). ¹³C-NMR (CDCl₃, 100 MHz) δ 173.8, 135.3 (4C), 133.2 (2C), 129.3 (2C), 127.3 (4C), 81.3, 65.6, 57.6, 51.1, 33.7, 30.8, 26.5 (3C), 24.7, 24.6, 18.8. MS (ESI) m/z: 451.3 (M+Na⁺).

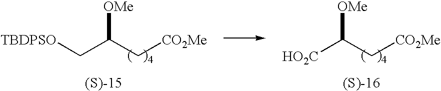

2-(S)-Methoxyheptanedioic acid 7-methyl ester [(S)-16]. Silylated diol (S)-15 (1.10 g, 2.33 mmol) was dissolved in anhydrous THF (23.3 mL) under argon and tetrabutylammonium fluoride (TBAF, 1.0 M solution in THF, 2.56 mL) was slowly added at 0° C. The reaction mixture was warmed to room temperature and monitored by TLC until complete consumption of the starting material. After 2 h the reaction was quenched with NH₄Cl (aq, sat.) and extracted with EtOAc. The organic phase was dried (MgSO₄) and concentrated under vacuum to afford a crude which was purified by flash chromatography (gradient 7:3 to 2:8 hexanes/EtOAc). Deprotected alcohol intermediate (0.36 g) was obtained in 80% yield as a colorless oil: [α]²⁰_{D} +17.3 (c 0.9, CHCl₃). ¹H-NMR (CDCl₃, 400 MHz) δ 3.68-3.65 (m, 1H), 3.67 (s, 3H), 3.48 (dd, J=11.6, 6.1 Hz, 1H), 3.39 (s, 3H), 3.25 (ddd, J=12.1, 6.0, 3.4 Hz, 1H), 2.32 (t, J=7.4 Hz, 2H), 2.17 (b, 1H), 1.68-1.32 (m, 6H). ¹³C-NMR (CDCl₃, 100 MHz) δ 173.7, 81.0, 63.3, 56.8, 51.2, 33.5, 29.6, 24.7, 24.5. MS (ESI) m/z: 191.1 (M+1).

This alcohol intermediate was dissolved in acetone (19 mL) and an aqueous 15% solution of NaHCO₃ (1.89 mL) was added at 0° C., followed by solid NaBr (39 mg, 0.38 mmol) and TEMPO (6 mg, 0.04 mmol). Trichloroisocyanuric acid (TCCA, 0.88 g, 3.78 mmol) was then added in portions during 30 min at 0° C. The mixture was allowed to reach room temperature and was stirred until completion (3 h), then 2-propanol was added. The mixture was filtered on Celite®, concentrated in vacuo, taken up in H₂O and extracted with EtOAc. The organic layers were dried (MgSO₄) and the solvent removed under reduced pressure to furnish the carboxylic acid (S)-16 as an amorphous white solid (0.35 g, 90% yield) which was used for the next reaction without further purification: [α]²⁰_{D} −26.0 (c 0.5, CHCl₃). ¹H-NMR (CDCl₃, 400 MHz) δ 9.20 (b, 1H), 3.81 (dd, J=7.2, 4.9 Hz, 1H), 3.67 (s, 3H), 3.44 (s, 3H), 2.34 (t, J=7.5 Hz, 2H), 1.80 (m, 2H), 1.67 (m, 2H), 1.48 (m, 2H). ¹³C-NMR (CDCl₃, 100 MHz) δ 177.1, 173.7, 79.5, 58.0, 51.2, 33.4, 31.7, 24.1 (2C). MS (ESI) m/z: 205.1 (M+1), 227.1 (M+Na⁺).

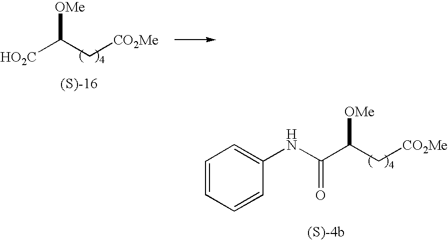

### 6-(S)-Methoxy-6-phenylcarbamoyl-hexanoic acid methyl ester [(S)-4b]

Anilide (S)-4b was prepared according to the general procedure (Method 1B) starting from carboxylic acid 16, aniline (0.23 mL, 2.55 mmol), EDC (1.71 g, 8.93 mmol), HOBt (0.45 g, 3.32 mmol) and DIEA (1.56 mL, 8.93 mmol) in anhydrous CH₂Cl₂ (9.0 mL). After flash chromatography (7:3 hexanes/EtOAc) pure anilide (S)-4b (0.38 g) was isolated in 79% yield as a pale yellow oil: [α]²⁰_{D} −72.6 (c 0.8, CHCl₃). ¹H- and ¹³C-NMR analyses were consistent to the ones reported for racemic 4b.

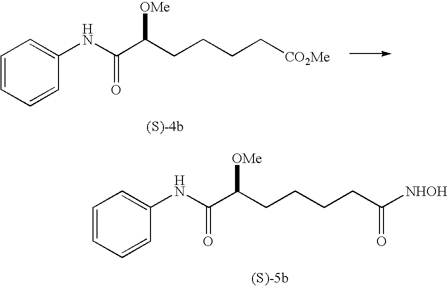

2-(S)-Methoxyheptanedioic acid 7-hydroxyamide 1-phenylamide [(S)-5b]. Hydroxamic acid (S)-5b was prepared according to the general procedure 7A starting from the corresponding methyl ester (S)-4b in 99% yield. A pale yellow oil: [α]²⁰_{D} −75.0° (c 0.1, CHCl₃). ¹H- and ¹³C-NMR analyses were consistent to the ones reported for racemic 5b. HPLC t_{R}=5.28 min. HRMS (ES+) C₁₄H₂₀N₂O₄ calcd for [MH]⁺ 281.14958. found 281.14932.

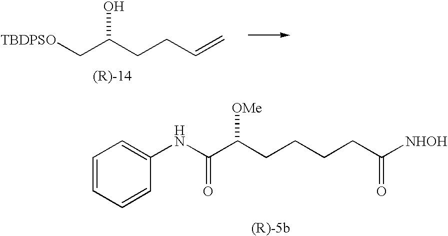

2-(R)-Methoxyheptanedioic acid 7-hydroxyamide 1-phenylamide [(R)-5b]. Hydroxamic acid (R)-5b was prepared starting from the alcohol (R)-14 (Dixon, D. J.; Ley, S. V.; Reynolds, D. J. Chem. Eur. J. Org. Chem. 2002, 8, 1621-1636) in accord to the procedure previously described for enantiomer (S)-5b. A pale yellow oil: [α]²⁰_{D} +74.7 (c 0.6, CHCl₃). ¹H- and ¹³C-NMR analyses were consistent to the ones reported for racemic 5b. HPLC t_{R}=4.00 min. HRMS (ES+) C₁₄H₂₀N₂O₄ calcd for [MH]⁺ 281.14958. found 281.14913.

### Example 6

### Preparation of Enantiopure 8 Carbon Long Chain Linear ω-Alkoxy Derivatives

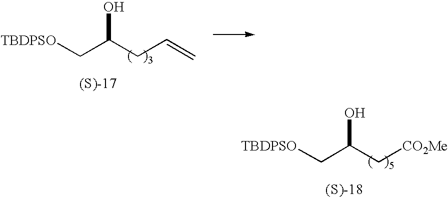

8-(tert-Butyldiphenylsilanyloxy)-7-(S)-hydroxyoctanoic acid methyl ester [(S)-18]. Olefin (S)-17 (Dixon, D. J.; Ley, S. V., Tate, E. W. J. Chem. Soc., Perkin Trans. 11998, 3125-3126) (2.50 g, 6.78 mmol) was subjected to a cross-metathesis reaction according to the general procedure 4A, coupling it with methyl acrylate (7.3 mL, 81.36 mmol) in the presence of Grubbs' catalyst 2^{nd} generation (172 mg, 0.203 mmol) in anhydrous CH₂Cl₂ (13.5 mL). After purification by flash chromatography (85:15 hexanes/EtOAc) the unsaturated methyl ester intermediate was recovered in 95% yield (2.75 g) as a colorless oil, ¹H-NMR (CDCl₃, 400 MHz) δ 7.69-7.67 (m, 4H), 7.48-7.40 (m, 6H), 6.95 (dt, J=15.6, 7.0 Hz, 1H), 6.82 (dt, J=15.6, 1.4 Hz, 1H), 3.74 (s, 3H), 3.66 (dd, J=10.1, 3.3 Hz, 1H), 3.49 (dd, J=10.1, 7.5 Hz, 2H), 2.21 (dd, J=13.0, 6.3 Hz, 2H), 2.10 (b, 1H), 1.62 (m, 1H), 1.45 (m, 3H), 1.09 (s, 9H). ¹³C-NMR (CDCl₃, 100 MHz) δ 166.7, 148.8, 135.2 (4C), 132.7 (2C), 129.5 (2C), 127.5 (4C), 120.8, 71.2, 67.5, 51.1, 31.7 (2C), 26.5 (3C), 23.6, 18.9. MS (ESI) m/z: 444.2 (M+18).

This olefin intermediate was hydrogenated according to the general procedure (Method 6A). After flash chromatography (8:2 hexanes/EtOAc), saturated ester (S)-18 (2.34 g) was obtained in 86% yield as a colorless oil: ¹H-NMR (CDCl₃, 400 MHz) δ 7.69-7.67 (m, 4H), 7.48-7.40 (m, 6H), 3.72 (m, 1H), 3.68 (s, 3H), 3.66 (m, 1H), 3.49 (dd, J=10.4, 7.5 Hz, 1H), 2.31 (t, J=7.5 Hz, 2H), 2.30 (b, 1H), 1.62 (m, 2H), 1.46-1.24 (m, 6H), 1.09 (s, 9H). ¹³C-NMR (CDCl₃, 100 MHz) δ 173.9, 135.2 (4C), 132.8 (2C), 129.5 (2C), 127.4 (4C), 71.5, 67.6, 51.1, 33.6, 32.2, 28.8, 26.5 (3C), 24.8, 24.5, 18.9. MS (ESI) m/z: 446.2 (M+18).

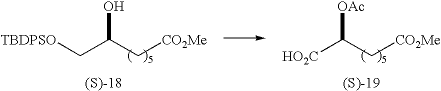

2-(S)-Acetoxyoctanedioic acid 8-methyl ester [(S)-19]. To a solution of alcohol (S)-18 (2.30 g, 5.37 mmol) in anhydrous pyridine (14.0 mL) under an argon atmosphere, acetic anhydride was added under stirring (0.61 mL, 6.4 mmol) followed by DMAP (66 mg, 0.54 mmol). After stirring overnight at room temperature the reaction was quenched with NH₄Cl (aq, sat.), and extracted with CH₂Cl₂. All the combined organic extracts were dried (MgSO₄), concentrated under vacuum and purified by flash chromatography (85:15 hexanes/EtOAc) which gave acetate intermediate (2.58 g) in 98% yield as a pale yellow oil: [α]²⁰_{D} −10.0 (c 0.6, CHCl₃). ¹H-NMR (CDCl₃, 400 MHz) δ 7.70-7.67 (m, 4H), 7.47-7.38 (m, 6H), 5.01 (m, 1H), 3.69 (s, 3H), 3.68 (m, 2H), 2.32 (t, J=7.4 Hz, 2H), 2.05 (s, 3H), 1.69-1.57 (m, 4H), 1.38-1.27 (m, 4H), 1.07 (s, 9H). ¹³C-NMR (CDCl₃, 100 MHz) δ 173.8, 170.4, 135.3 (2C), 135.2 (2C), 133.1, 133.0, 129.4, 129.3, 127.3 (4C), 73.9, 64.6, 51.1, 33.6, 29.9, 28.6, 26.4 (3C), 24.5, 24.4, 20.8, 18.9. MS (ESI) m/z: 471.3 (M+1), 493.3 (M+Na⁺).

A solution of this fully protected diol intermediate (2.30 g, 4.89 mmol) in THF (49.0 mL) was treated with a solution of TBAF/AcOH (1:1, ca 1M in THF, 5.69 mL, 5.38 mmol) at 0° C. under argon. After warming to room temperature the reaction mixture was stirred at room temperature and monitored by TLC until complete conversion of the starting material. After 3 h the reaction was judged complete, quenched with NH₄Cl (aq, sat.) and extracted with CH₂Cl₂. The organic phase was dried (MgSO₄), concentrated under reduced pressure and purified by flash chromatography (gradient 1:1 to 8:2 EtOAc/hexanes), which furnished 1.11 g of desilylated alcohol intermediate as a colorless oil (97% yield): [α]²⁰_{D} ˜0 (c 1.6, CHCl₃). ¹H-NMR (CDCl₃, 400 MHz) δ 4.91 (ddd, J=13.2, 6.3, 3.3 Hz, 1H), 3.72 (dd, J=12.0, 3.3 Hz, 1H), 3.68 (s, 3H), 3.63 (dd, J=12.0, 6.2 Hz, 1H), 2.32 (t, J=7.4 Hz, 2H), 2.10 (s, 3H), 2.04 (b, 1H), 1.67-1.58 (m, 4H), 1.38-1.31 (m, 4H). ¹³C-NMR (CDCl₃, 100 MHz) δ 173.8, 171.1, 75.0, 64.3, 51.2, 33.5, 29.9, 28.5, 24.6, 24.3, 20.8. MS (ESI) m/z: 233.2 (M+1), 255.1 (M+Na⁺).

This alcohol intermediate was dissolved in acetone (48.5 mL) and an aqueous 15% solution of NaHCO₃ (14.1 mL) was added at 0° C., followed by solid NaBr (99 mg, 0.96 mmol) and TEMPO (15 mg, 0.10 mmol). TCCA (2.22 g, 9.56 mmol) was then added in portions during 30 min at 0° C. The mixture was allowed to reach room temperature and was stirred until completion (3 h), then 2-propanol was added. The mixture was filtered on Celite®, concentrated in vacuo, taken up in H₂O and extracted with EtOAc. The organic layers were dried (MgSO₄) and the solvent removed under reduced pressure to furnish carboxylic acid (S)-19 as an amorphous white solid (1.17 g, 99% yield) which was used for the next reaction without further purification: [α]²⁰_{D} −11.2 (c 1.3, CHCl₃). ¹H-NMR (CDCl₃, 400 MHz) δ 8.65 (b, 1H), 5.00 (t, J=6.8 Hz, 1H), 3.68 (s, 3H), 2.33 (t, J=7.4 Hz, 2H), 2.15 (s, 3H), 1.87 (m, 2H), 1.64 (m, 2H), 1.45 (m, 2H), 1.36 (m, 2H). ¹³C-NMR (CDCl₃, 100 MHz) δ 175.0, 173.9, 170.4, 71.4, 51.2, 33.5, 30.3, 28.2, 24.4, 24.2, 20.2. MS (ESI) m/z: 247.1 (M+1).

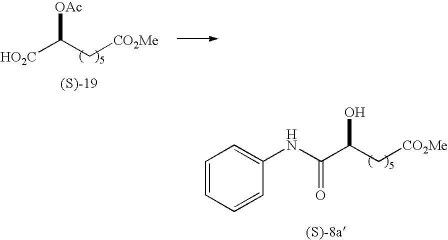

7-(S)-Hydroxy-7-phenylcarbamoyl heptanoic acid methyl ester [(S)-8a′]. Carboxylic acid 19 and aniline (0.65 mL, 7.13 mmol) in anhydrous CH₂Cl₂ (24 mL) were coupled according to the general procedure (Method 1B). After purification by flash chromatography (7:3 hexanes/EtOAc) the pure anilide intermediate (1.07 g) was isolated in 70% yield as a pale yellow oil: [α]²⁰_{D} −32.6 (c 0.43, CHCl₃). ¹H-NMR (CDCl₃, 400 MHz) δ 7.88 (s, 1H), 7.55 (d, J=7.8 Hz, 2H), 7.35 (t, J=7.7 Hz, 2H), 7.15 (t, J=7.4 Hz, 1H), 5.28 (t, J=6.7 Hz, 1H), 3.68 (s, 3H), 2.32 (t, J=7.4 Hz, 2H), 2.23 (s, 3H), 1.95 (m, 2H), 1.64 (m, 2H), 1.45-1.31 (m, 4H). ¹³C-NMR (CDCl₃, 100 MHz) δ 173.8, 169.4, 167.5, 136.6, 128.7 (2C), 124.4, 119.7 (2C), 73.7, 51.2, 33.5, 31.2, 28.2, 24.2, 24.0, 20.7. MS (ESI) m/z: 322.1 (M+1).

To this intermediate dissolved in anhydrous MeOH (23.6 mL) solid KCN (108 mg, 1.67 mmol) was added under an argon atmosphere and the resulting mixture was stirred at room temperature for 2 h. The solvent was removed under vacuum and the crude was purified by flash chromatography (gradient 6:4 to 3:7 hexanes/EtOAc), which yielded 0.92 g of the free alcohol (S)-8a′ (99%) as a pale yellow solid: [α]²⁰_{D} −37.1 (c 0.3, CHCl₃). ¹H- and ¹³C-NMR analyses were consistent to the ones reported for racemic 8a′.

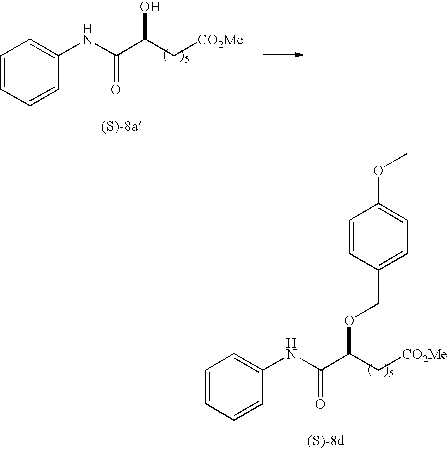

(S)-7-(4-Methoxybenzyloxy)-7-phenylcarbamoyl heptanoic acid methyl ester [(S)-8d]. Ether (S)-8d was prepared in 39% yield from alcohol (S)-8a′ following to the general procedure (Method 2B). A pale yellow oil, [α]²⁰_{D} −52.5 (c 0.8, CHCl₃). ¹H- and ¹³C-NMR analyses were consistent to the ones reported for racemic 8d.

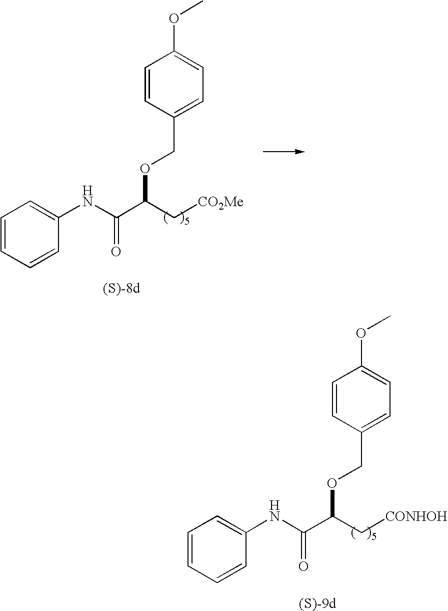

2-(S)-(4-Methoxybenzyloxy)octanedioic acid 8-hydroxyamide 1-phenylamide [(S)-9d]. Hydroxamic acid (S)-9d was obtained from the corresponding methyl ester (S)-8d in 99% yield following the general procedure 6A. A pale yellow oil: [α]²⁰_{D} −50.0 (c 0.05, CHCl₃). ¹H- and ¹³C-NMR analyses were consistent to the ones reported for racemic 9d. HPLC t_{R}=5.71 min. HRMS (ES+) C₂₂H₂₈N₂O₅ calcd for [MH]⁺ 401.20710. found 401.20572.

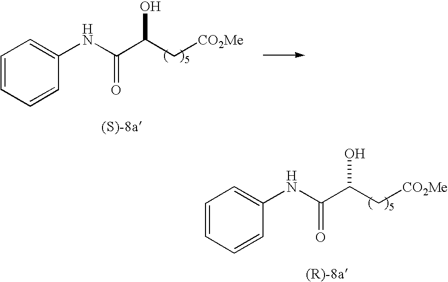

7-(R)-Hydroxy-7-phenylcarbamoylheptanoic acid methyl ester [(R)-8a′]. To a solution of alcohol (S)-8a′ (0.50 g, 1.79 mmol), p-nitrobenzoic acid (0.45 g, 2.69 mmol), and Ph₃P (0.70 g, 2.69 mmol) in anhydrous toluene (22.4 mL), DIAD (0.53 mL, 2.69 mmol) was added dropwise while stirring at 0° C. under argon. The reaction mixture was then warmed to room temperature and allowed to react until complete conversion of the starting material. After 2 h the solvent was removed under reduced pressure and the residue purified by flash chromatography (6:4 hexanes/EtOAc) to give the intermediate p-nitrobenzoate (0.64 g) as a yellow solid: [α]²⁰_{D} −46.0 (c 0.1, CHCl₃). ¹H-NMR (CDCl₃, 400 MHz) δ 8.35 (d, J=8.9 Hz, 2H), 8.29 (d, J=8.9 Hz, 2H), 7.92 (s, 1H), 7.54 (d, J=7.6 Hz, 2H), 7.34 (t, J=7.6 Hz, 2H), 7.15 (t, J=7.4 Hz, 1H), 5.48 (t, J=6.4 Hz, 1H), 3.68 (s, 3H), 2.33 (t, J=7.4 Hz, 2H), 2.13 (m, 2H), 1.66 (m, 2H), 1.54 (m, 2H), 1.43 (m, 2H). ¹³C-NMR (CDCl₃, 100 MHz) δ 173.8, 166.8, 163.6, 150.5, 136.5, 134.1, 130.6 (2C), 128.7 (2C), 124.6, 123.5 (2C), 119.7 (2C), 75.1, 51.2, 33.4, 31.1, 28.1, 24.2, 24.1.

To this intermediate dissolved in anhydrous MeOH (10.0 mL) solid KCN (49 mg, 0.75 mmol) was added under an argon atmosphere and the resulting mixture was stirred at room temperature for 1 h. The solvent was removed under vacuum, and the crude was purified by flash chromatography (gradient 6:4 to 4:6 hexanes/EtOAc) which yielded 0.42 g of enantiomer (R)-8a′ (83% yield over two steps) as a pale yellow solid: [α]²⁰_{D} +37.5 (c 0.2, CHCl₃). ¹H- and ¹³C-NMR analyses were consistent to the ones reported for racemic and (S)-enantiopure 8a′.

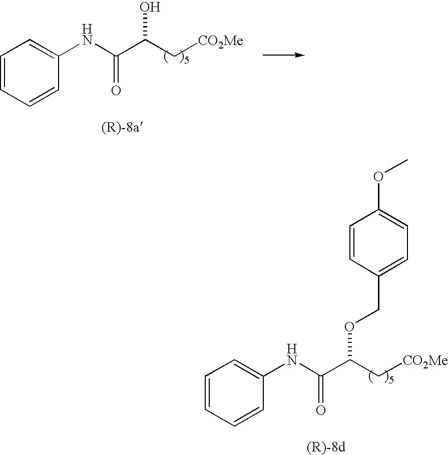

(R)-7-(4-Methoxybenzyloxy)-7-phenylcarbamoyl heptanoic acid methyl ester [(R)-8d]. Ether (R)-8d was prepared in 39% yield from alcohol (R)-8a′ following to the general procedure (Method 2B). A pale yellow oil: [α]²⁰_{D} +51.7 (c 0.2, CHCl₃). ¹H- and ¹³C-NMR analyses were consistent to the ones reported for racemic and (S)-enantiopure 8d.

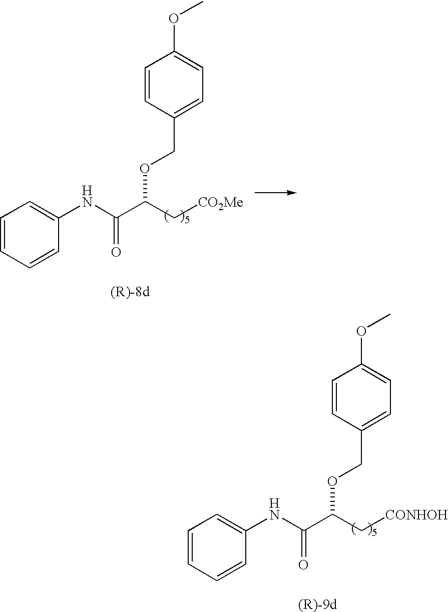

2-(R)-(4-Methoxybenzyloxy)octanedioic acid 8-hydroxyamide 1-phenylamide [(R)-9d]. Hydroxamic acid (R)-9d was obtained from the corresponding methyl ester (R)-8d in 99% yield following the general procedure 6A. A pale yellow oil: [α]²⁰_{D} +47.3 (c 0.1, CHCl₃). ¹H- and ¹³C-NMR analyses were consistent to the ones reported for racemic and (S)-enantiopure 9d. HPLC t_{R}=7.72 min. HRMS (ES+) C₂₂H₂₈N₂O₅ calcd for [MH]⁺ 401.20710. found 401.20561.

### Example 7

### Preparation of Macrocyclic Hydroxamic Acids Containing an Aliphatic Tether

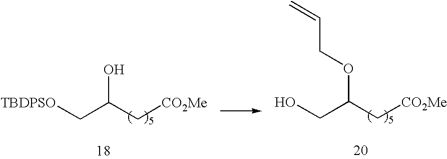

(±)-7-Allyloxy-8-hydroxyoctanoic acid methyl ester (20). To a stirred solution of racemic alcohol 18 (2.20 g, 5.13 mmol) in cyclohexane (35.0 mL) at room temperature under an argon atmosphere, a freshly prepared solution of allyl trichloroacetimidate (Faul, M. M.; Winneroski, L. L.; Krumrich, C. A.; Sullivan, K. A.; Gillig, J. R.; Neel, D. A.; Rito, C. J.; Jirousek, M. R. J. Org. Chem. 1998, 63, 1961-1973) (1 M solution in cyclohexanes, 10.26 mL) was added, followed by trifluoromethanesulfonic acid (TfOH, 0.11 mL, 50 μL/g alcohol). After stirring for 72 h the reaction mixture was filtered through a pad of Celite® to remove the precipitate, the precipitate was washed with petroleum ether, and the filtrate concentrated in vacuo. The resulting crude residue was purified by flash chromatography (gradient 9:1 to 1:1 hexanes/EtOAc) which gave 1.78 g of O-allyl ether intermediate (74% yield) as a colorless oil: ¹H-NMR (CDCl₃, 400 MHz) δ 7.29-7.70 (m, 4H), 7.47-7.40 (m, 6H), 5.92 (ddt, J=17.2, 10.4, 5.7 Hz, 1H), 5.26 (dd, J=17.2, 1.7 Hz, 1H), 5.16 (dd, J=10.3, 1.6 Hz, 1H), 4.15 (dd, J=12.7, 5.5 Hz, 1H), 4.00 (dd, J=12.7, 5.9 Hz, 1H), 3.71 (dd, J=10.6, 5.8 Hz, 1H), 3.69 (s, 3H), 3.61 (dd, J=10.6, 4.9 Hz, 1H), 3.41 (m, 1H), 2.33 (t, J=7.5 Hz, 2H), 1.68-1.58 (m, 2H), 1.57-1.42 (m, 2H), 1.34-1.27 (m, 4H), 1.09 (s, 9H). ¹³C-NMR (CDCl₃, 100 MHz) δ 173.9, 135.3 (4C), 134.1, 133.2 (2C), 129.3 (2C), 127.3 (4C), 116.2, 79.2, 70.9, 65.8, 51.1, 33.7, 31.2, 28.9, 26.5 (3C), 24.7, 24.5, 18.8. MS (ESI) m/z: 486.3 (M+18).

This fully protected intermediate was dissolved in anhydrous THF (36.0 mL) under argon and tetrabutylammonium fluoride (TBAF, 1.0 M solution in THF, 4.18 mL) was slowly added at 0° C. The reaction mixture was warmed to room temperature and monitored by TLC until complete consumption of the starting material. After 2 h the reaction was quenched with NH₄Cl (aq, sat.) and extracted with EtOAc. The organic phase was dried (MgSO₄) and concentrated in vacuo to afford a crude which was purified by flash chromatography (gradient 7:3 to 1:1 hexanes/EtOAc). Alcohol 20 (0.86 g) was obtained in 99% yield as a colorless oil: H-NMR (CDCl₃, 400 MHz) δ 5.92 (ddt, J=17.2, 10.4, 5.7 Hz, 1H), 5.28 (dd, J=17.2, 1.6 Hz, 1H), 5.18 (dd, J=10.3, 1.5 Hz, 1H), 4.05 (dt, J=6.6, 1.2 Hz, 2H), 3.66 (s, 3H), 3.65 (dd, J=11.4, 3.4 Hz, 1H), 3.49 (dd, J=11.5, 6.2 Hz, 1H), 3.41 (ddd, J=12.2, 6.0, 3.4 Hz, 1H), 2.31 (t, J=7.4 Hz, 2H), 1.94 (b, 1H), 1.63 (m, 2H), 1.55 (m, 1H), 1.47 (m, 1H), 1.38-1.28 (m, 4H). ¹³C-NMR (CDCl₃, 100 MHz) δ 173.8, 134.6, 116.6, 79.1, 70.2, 63.8, 51.1, 33.6, 30.3, 28.9, 24.7, 24.4. MS (ESI) m/z: 231.1 (M+1).

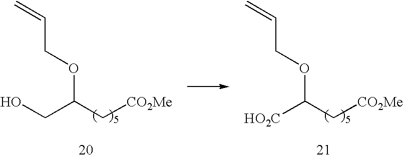

(±)-2-Allyloxyoctanedioic acid 8-methyl ester (21). To a solution of oxalyl chloride (0.97 mL, 11.07 mmol) in CH₂Cl₂ (64.0 mL) at −78° C. under an argon atmosphere, a solution of DMSO (1.05 mL, 14.76 mmol) in CH₂Cl₂ (11.0 mL) was added dropwise. After 10 min a solution of alcohol 20 (0.85 g, 3.69 mmol) in CH₂Cl₂ (20.0 mL) was added at the same temperature. After 1 h Et₃N (5.14 mL, 36.90 mmol) was added, stirring was continued at −78° C. for 30 min, then the reaction was allowed to reach room temperature over a period of 1 h. NH₄Cl (aq, sat.) was added, and the mixture was extracted with CH₂Cl₂. The organic phase was dried (MgSO₄), concentrated in vacuo and the resulting residue purified by flash chromatography (7:3 hexanes/EtOAc) which furnished the pure aldehyde intermediate (0.83 g) in 99% yield as a colorless oil: ¹H-NMR (CDCl₃, 400 MHz) δ 9.66 (d, J=2.1 Hz, 1H), 5.92 (ddt, J=17.2, 10.4, 5.7 Hz, 1H), 5.32 (dd, J=17.2, 1.5 Hz, 1H), 5.25 (dd, J=10.3, 1.2 Hz, 1H), 4.16 (dd, J=12.6, 5.6 Hz, 1H), 4.03 (dd, J=12.6, 5.9 Hz, 1H), 3.71 (dt, J=6.5, 2.0 Hz, 1H), 3.61 (s, 3H), 2.32 (t, J=7.4 Hz, 2H), 1.71-1.61 (m, 4H), 1.45 (m, 2H), 1.34 (m, 2H). ¹³C-NMR (CDCl₃, 100 MHz) δ 203.4, 173.7, 133.5, 117.8, 83.0, 71.2, 51.1, 33.5, 29.5, 28.5, 24.3, 24.1. MS (ESI) m/z: 229.1 (M+1).

A solution of this aldehyde intermediate in acetone (36.0 mL) was treated at 0° C. with Jones reagent (4.80 mL, prepared from 27.0 g of chromium (VI) oxide, 23.0 mL of H₂SO₄, and 75.0 mL of water). After 15 min at 0° C. the reaction was quenched with MeOH (180 mL), and partitioned between EtOAc and water. The organic layer was washed with 10% NaHSO₄, 10% Na₂S₂O₃, water, dried (MgSO₄) and concentrated in vacuo to afford the carboxylic acid 21 as an amorphous white solid (0.88 g, 99% yield) which was used without further purification: ¹H-NMR (CDCl₃, 400 MHz) δ 9.20 (b, 1H), 5.92, (ddt, J=17.0, 10.2, 5.8 Hz, 1H), 5.33 (d, J=17.2 Hz, 1H), 5.25 (d, J=10.3 Hz, 1H), 4.19 (dd, J=12.5, 5.5 Hz, 1H), 3.99 (m, 2H), 3.68 (s, 3H), 2.32 (t, J=7.4 Hz, 2H), 1.81 (m, 2H), 1.66 (m, 2H), 1.48 (m, 2H), 1.36 (m, 2H). ¹³C-NMR (CDCl₃, 100 MHz) δ 177.4, 173.8, 133.2, 118.1, 77.0, 71.3, 51.2, 33.6, 31.9, 28.4, 24.3 (2C). MS (ESI) m/z: 245.1 (M+1).

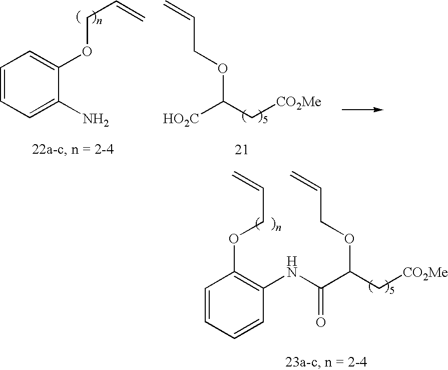

(±)-7-Allyloxy-7-(2-but-3-enyloxyphenylcarbamoyl)heptanoic acid methyl ester (23a), (±)-7-Allyloxy-7-(2-pent-4-enyloxyphenylcarbamoyl) heptanoic acid methyl ester (23b), (±)-7-Allyloxy-7-(2-hex-5-enyloxyphenylcarbamoyl)heptanoic acid methyl ester (23c). Anilide 23a was prepared according to the general procedure (Method 1B) starting from carboxylic acid 21 (0.25 g, 1.02 mmol), aniline 22a (Beckwith, A. L. J.; Gara, W. B.; J. Chem. Soc., Perk. Trans. 11975, 593-600) (0.28 g, 1.73 mmol), EDC (0.68 g, 3.57 mmol), HOBt (0.18 g, 1.35 mmol) and DIEA (0.62 mL, 3.57 mmol) in anhydrous CH₂Cl₂ (5.0 mL). Purification by flash chromatography (gradient 9:1 to 7:3 hexanes/EtOAc) furnished anilide 23a (0.30 g) as a pale yellow oil in 75% yield: ¹H-NMR (CDCl₃, 400 MHz) δ 9.06 (s, 1H), 8.43 (dd, J=7.9, 1.6 Hz, 1H), 7.06 (dt, J=7.8, 1.7 Hz, 1H), 6.98 (dt, J=7.7, 1.3 Hz, 1H), 6.90 (dd, J=8.0, 1.3 Hz, 1H), 5.96 (ddt, J=17.2, 10.5, 5.6 Hz, 1H), 5.92 (ddt, J=17.2, 10.3, 6.7, 1H), 5.36 (dd, J=17.2, 1.6 Hz, 1H), 5.26 (dd, J=10.4, 1.3 Hz, 1H), 5.21 (dd, J=17.2, 2.8 Hz, 1H), 5.14 (dd, J=10.2, 1.4 Hz, 1H), 4.19-4.04 (m, 4H), 3.91 (dd, J=7.0, 4.4 Hz, 1H), 3.68 (s, 3H), 2.61 (ddd, J=13.2, 6.6, 1.1 Hz, 2H), 2.32 (t, J=15.4 Hz, 2H), 1.82 (m, 2H), 1.65 (m, 2H), 1.47 (m, 2H), 1.34 (m, 2H). ¹³C-NMR (CDCl₃, 100 MHz) δ 173.8, 170.5, 147.1, 133.4, 126.8, 123.5, 120.7, 119.3, 117.5, 117.1, 110.5, 80.0, 71.3, 67.2, 51.1, 33.6, 33.3, 32.5, 28.6, 24.4, 24.3. MS (ESI) m/z: 390.2 (M+1).

Anilide 23b was prepared according to the general procedure (Method 1B) starting from carboxylic acid 2i (0.25 g, 1.02 mmol), aniline 22b (Beckwith, A. L. J.; Meijs, G. F. J. Org. Chem. 1987, 52, 1922-1930; alternatively, 22b was prepared from o-nitrophenol and 5-penten-1-ol in 94% overall yield following a two-step sequence including the general procedure 2C2 followed by Method 3A2) (0.31 g, 1.73 mmol), EDC (0.68 g, 3.57 mmol), HOBt (0.18 g, 1.35 mmol) and DIEA (0.62 mL, 3.57 mmol) in anhydrous CH₂Cl₂ (5.0 mL). Purification by flash chromatography (gradient 9:1 to 75:25 hexanes/EtOAc) furnished anilide 23b (0.28 g) as a pale yellow oil in 69% yield: ¹H-NMR (CDCl₃, 400 MHz) δ 9.10 (s, 1H), 8.43 (dd, J=7.9, 1.5 Hz, 1H), 7.06 (dt, J=7.8, 1.6 Hz, 1H), 6.98 (dt, J=7.6, 1.2 Hz, 1H), 6.89 (dd, J=8.1, 1.2 Hz, 1H), 6.04 (ddt, J=17.2, 10.4, 5.6 Hz, 1H), 5.86 (ddt, J=17.0, 10.3, 6.6 Hz, 1H), 5.37 (dd, J=17.2, 1.5 Hz, 1H), 5.25 (dd, J=10.4, 1.4 Hz, 1H), 5.09 (dd, J=17.1, 1.7 Hz, 1H), 5.04 (dd, J=11.9, 1.7 Hz, 1H), 4.19-4.01 (m, 4H), 3.91 (dd, J=6.8, 4.4 Hz, 1H), 3.67 (s, 3H), 2.31 (t, J=7.5 Hz, 2H), 2.70 (m, 2H), 1.95 (m, 2H), 1.85 (m, 2H), 1.65 (m, 2H), 1.47 (m, 2H), 1.34 (m, 2H). ¹³C-NMR (CDCl₃, 100 MHz) δ 173.8, 170.4, 147.2, 137.1, 133.3, 126.8, 123.5, 120.6, 119.2, 117.5, 115.1, 110.5, 80.0, 71.3, 67.3, 51.1, 33.6, 32.5, 29.7, 28.6, 28.0, 24.4, 24.2. MS (ESI) m/z: 404.2 (M+1).

Anilide 23c was prepared according to the general procedure (Method 1B) starting from carboxylic acid 21 (0.25 g, 1.02 mmol), aniline 22c (0.33 g, 1.73 mmol), EDC (0.68 g, 3.57 mmol), HOBt (0.18 g, 1.35 mmol) and DIEA (0.62 mL, 3.57 mmol) in anhydrous CH₂Cl₂ (5.0 mL). Purification by flash chromatography (gradient 9:1 to 8:2 hexanes/EtOAc) furnished anilide 23c (0.33 g) as a pale yellow oil in 78% yield: ¹H-NMR (CDCl₃, 400 MHz) δ 9.09 (s, 1H), 8.43 (d, J=7.8 Hz, 1H), 7.06 (t, J=7.7 Hz, 1H), 6.98 (t, J=7.8 Hz, 1H), 6.89 (d, J=8.0 Hz, 1H), 5.95 (ddt, J=16.8, 10.7, 5.7 Hz, 1H), 5.84 (ddt, J=17.1, 10.0, 6.7 Hz, 1H), 5.38 (d, J=17.1 Hz, 1H), 5.26, (d, J=10.4 Hz, 1H), 5.06, (d, J=17.1 Hz, 1H), 5.01 (d, J=10.2 Hz, 1H), 4.16 (m, 1H), 4.06 (m, 3H), 3.91 (t, J=5.9 Hz, 1H), 3.68 (s, 3H), 2.32 (t, J=7.5 Hz, 2H), 2.15 (dd, J=13.9, 6.9 Hz, 2H), 1.89-1.78 (m, 4H), 1.69-1.60, (m, 4H), 1.51-1.43 (m, 2H), 1.42-1.32 (m, 2H). ¹³C-NMR (CDCl₃, 100 MHz) δ 173.8, 170.5, 147.3, 137.9, 133.3, 123.5, 120.5, 119.2, 117.4, 114.6, 110.4, 81.5, 80.0, 71.3, 67.9, 51.1, 33.6, 33.0, 32.5, 28.6, 28.3, 24.9, 24.4, 24.3. MS (ESI) m/z: 418.3 (M+1).

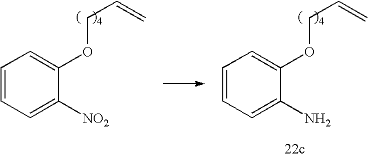

2-Hex-5-enyloxyphenylamine (22c). Aniline 22c was prepared starting from the corresponding 1-nitro-2-hex-5-enyloxybenzene which in turn was synthesized according to the general procedure (Method 2C1) starting from o-nitrophenol (1.00 g, 7.19 mmol), 6-bromo-1-hexene (7.76 mmol) and Na₂CO₃ (0.46 g, 4.31 mmol) in H₂O (2.9 mL) in 61% yield. An orange oil: ¹H-NMR (CDCl₃, 400 MHz): δ 7.84 (dd, J=8.1, 1.6 Hz, 1H), 7.53 (dt, J=7.7, 1.7 Hz, 1H), 7.08 (d, J=8.4 Hz, 1H), 7.02 (dt, J=7.6, 1.0 Hz, 1H), 7.84 (ddt, J=17.0, 10.3, 6.7 Hz, 1H), 5.06 (dd, J=17.1, 1.7 Hz, 1H), 4.00 (dd, J=10.2, 0.8 Hz, 1H), 4.12 (t, J=6.3 Hz, 2H), 2.15 (dd, J=14.1, 7.1 Hz, 2H), 1.87 (dt, J=6.8, 6.4 Hz, 2H), 1.62 (m, 2H). ¹³C-NMR (CDCl₃, 100 MHz) δ 152.1, 138.0, 133.6 (2C), 125.2, 119.7, 114.6, 114.0, 69.0, 32.9, 28.0, 24.7.

This nitrobenzene intermediate was reduced to aniline derivatives 22c according to the general procedure (acid/base work-up). Alternatively, 22c was prepared from o-nitrobenzene in 93% overall yield following a two-step sequence including the general procedure 2C2 followed by Method 3A2. An orange oil (61% yield): ¹H-NMR (CDCl₃, 400 MHz) δ 6.79 (m, 2H), 6.73 (m, 2H), 5.86 (ddt, J=17.0, 10.3, 6.6 Hz, 1H), 5.05 (dd, J=17.1, 1.9, 1H), 5.00 (d, J=9.0 Hz, 1H), 4.02 (t, J=6.4 Hz, 2H), 3.82 (b, 2H), 2.16 (dd, J=14.1, 6.7 Hz, 2H), 1.85 (m, 2H), 1.61 (m, 2H). ¹³C-NMR (CDCl₃, 100 MHz) δ 146.2, 138.2, 135.9, 120.6, 118.1, 114.7, 114.4, 110.1, 67.6, 33.1, 28.5, 25.1. MS (ESI) m/z: 192.1 (M+1).

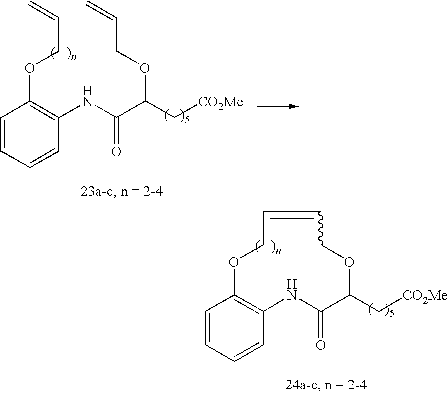

(±)-(E/Z)-6-(6-Oxo-6,7,12,13-tetrahydro-5H,9H-8,14-dioxa-5-azabenzo-cyclododecen-7-yl)hexanoic acid methyl ester (24a), (±)-(E/Z)-6-(6-Oxo-6,7,9,12,13,14-hexahydro-5H-8,15-dioxa-5-azabenzocyclotridecen-7-yl) hexanoic acid methyl ester (24b), (±)-(E/Z)-6-(6-Oxo-6,7,12,13,14,15-hexahydro-5H,9H-8,16-dioxa-5-azabenzocyclotetradecen-7-yl)hexanoic acid methyl ester (24c). Macrocyclic olefin 24a was prepared according to the general procedure 5A starting from the corresponding diolefin precursor 23a. After purification by flash chromatography (gradient 8:2 to 7:3 hexanes/EtOAc) pure macrocycle 24a was obtained in 78% yield as a pale yellow oil in an unseparable 45:54 cis/trans mixture: HPLC t_{R}=6.82 min, cis isomer; 7.03 min, trans isomer. ¹H-NMR (C₆D₆, 400 MHz), 2 isomers: δ 9.49 (s, 1H, cis isomer), 9.23 (s, 1H, trans isomer), 9.10 (dd, J=8.1, 1.5 Hz, 1H, trans isomer), 8.95 (dd, J=8.0, 1.5 Hz, 1H, cis isomer), 7.06 (m, 2H, cis+trans isomers) 6.97 (m, 2H, cis+trans isomers), 6.87 (dd, J=8.1, 1.3 Hz, trans isomer), 6.80 (dd, J=8.0, 1.3 Hz, 1H, cis isomer), 5.83 (ddd, J=10.8, 4.7 Hz, 1H, cis isomer), 5.64 (ddd, J=14.7, 7.2 Hz, 1H, trans isomer), 5.38 (m, 2H, cis+trans isomers), 4.29 (dd, J=11.5, 7.0 Hz, 1H, cis isomer), 3.93-3.81 (m, 6H, cis+trans isomers), 3.72-3.64 (m, 2H, cis+trans isomers), 3.53 (m, 1H, cis isomer), 3.46 (s, 6H, cis+trans isomers), 3.41 (dd, J=11.7, 7.5 Hz, 1H, trans isomer), 2.17 (t, J=7.5 Hz, 2H, trans isomer), 2.16 (t, J=7.8 Hz, 2H, cis isomer), 2.09-2.17 (m, 8H, cis+trans isomers), 1.66-1.37 (m, 10H, cis+trans isomers), 1.30-1.22 (cis+trans isomers). ¹³C-NMR (CDCl₃, 100 MHz), 2 isomers: δ 173.8 (2C), 171.9, 171.0, 149.3, 147.9, 134.3, 131.5, 130.6, 129.4, 128.9, 126.7, 124.4, 124.0, 122.8, 122.5, 120.9, 120.4, 117.4, 117.1, 83.7, 80.4, 73.5, 72.8, 71.4, 65.1, 51.1 (2C), 33.9, 33.6 (2C), 33.0, 32.3, 28.6, 28.5, 24.9, 24.8 (2C), 24.4 (2C). MS (ESI) m/z: 362.1 (M+1).

Macrocyclic olefin 24b was prepared according to the general procedure 5A starting from the corresponding diolefin precursor 23b. After purification by flash chromatography (gradient 85:15 to 7:3 hexanes/EtOAc) pure macrocycle 24b was obtained in 98% yield as a colorless oil in a10:90 cis/trans unseparable mixture: ¹H-NMR (CDCl₃, 400 MHz), trans isomer: δ 9.24 (s, 1H), 8.49 (dd, J=8.0, 1.3 Hz, 1H), 7.04 (dt, J=7.8, 1.4 Hz, 1H), 6.98, (t, J=7.7 Hz, 1H), 6.85 (d, J=8.0 Hz, 1H), 6.10 (ddd, J=14.9, 7.1 Hz, 1H), 5.67 (dt, J=15.3, 5.0 Hz, 1H), 4.39 (m, 2H), 3.92 (m, 2H), 3.68 (s, 3H), 3.67 (m, 1H), 2.33 (t, J=7.5 Hz, 2H), 2.23-1.89 (m, 4H), 1.75 (m, 2H), 1.67 (m, 2H), 1.54 (m, 2H), 1.38 (m, 2H). ¹³C-NMR (CDCl₃, 100 MHz) δ 173.8, 171.3, 146.8, 135.8, 127.1, 124.4, 123.3, 120.5, 118.9, 109.8, 82.2, 71.2, 68.9, 51.1, 33.7, 33.2, 31.1, 28.7, 28.5, 25.0, 24.5. MS (ESI) m/z: 376.1 (M+1).

Macrocyclic olefin 24c was prepared according to the general procedure 5A starting from the corresponding diolefin precursor 23c. After purification by flash chromatography (gradient 9:1 to 7:3 hexanes/EtOAc) pure macrocycle 24c was obtained in 99% yield as a pale yellow oil in an unseparable 46:54 cis/trans mixture: HPLC t_{R}=8.07 min, cis isomer; 8.24 min, trans isomer. ¹H-NMR (CDCl₃, 400 MHz), 2 isomers: δ 9.26 (s, 1H, trans isomer), 9.06 (s, 1H, cis isomer), 8.49 (dd, J=8.0, 1.6 Hz, 1H, trans isomer), 8.40 (dd, J=7.9, 1.6 Hz, 1H, cis isomer), 7.10-7-95 (m, 4H, cis+trans isomers), 6.91 (dd, J=8.0, 1.2 Hz, 1H, trans isomer), 6.85 (dd, J=8.0, 1.2 Hz, 1H, cis isomer), 5.95 (dt, J=15.2, 7.0 Hz, 1H, trans isomer), 5.80-5.73 (m, 2H, cis+trans isomers), 5.68 (dt, J=10.7, 3.8 Hz, 1H, cis isomer), 4.45 (t, J=9.9 Hz, 1H, cis isomer), 4.37 (dd, J=11.9, 4.7 Hz, 1H, trans isomer), 4.17 (m, 1H, cis isomer), 4.04 (t, J=9.1 Hz, 1H, trans isomer), 3.98 (m, 2H, cis+trans isomers), 3.87 (m, 2H, cis+trans isomers), 3.81 (dd, J=8.3, 3.4 Hz, 1H, cis isomer), 3.76 (dd, J=9.7, 4.8 Hz, 1H, trans isomer), 3.68 (s, 6H, cis+trans isomers), 2.33 (t, J=7.5 Hz, 4H, cis+trans isomers), 2.17-2.03 (m, 4H, cis+trans isomers), 2.00-1.36 (m, 24H, cis+trans isomers). ¹³C-NMR (CDCl₃, 100 MHz) δ 173.8 (2C), 171.1, 170.4, 147.6 (2C), 136.2, 133.7, 126.2,8, 126.2, 124.6, 123.6, 123.5, 123.2, 120.7, 120.4, 119.5, 119.1, 110.8, 110.1, 81.8, 81.3, 72.3, 70.5, 69.0, 66.3, 51.1 (2C), 33.7 (2C), 33.3, 33.2 (2C), 31.1, 28.6 (2C), 26.1, 25.9, 25.7, 25.6, 24.9, 24.8 24.6 (2C). MS (ESI) m/z: 390.2 (M+1).

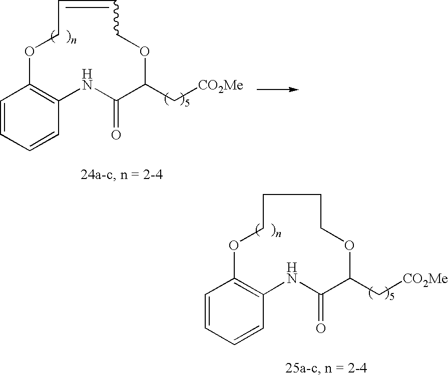

(±)-6-(6-Oxo-6,7,9,10,11,12-hexahydro-5H-8,13-dioxa-5-azabenzocyclo-undecen-7-yl)hexanoic acid methyl ester (25a), (±)-6-(6-Oxo-6,7,10,11,12,13-hexahydro-5H,9H-8,14-dioxa-5-azabenzocyclododecen-7-yl) hexanoic acid methyl ester (25b), (±)-6-(6-Oxo-6,7,9,10,11,12,13,14-octahydro-5H-8,15-dioxa-5-azabenzocyclotridecen-7-yl)hexanoic acid methyl ester (25c). Macrocyclic olefin 24a (0.15 g, 0.42 mmol) was hydrogenated according to the general procedure (Method 6A). After flash chromatography (7:3 hexanes/EtOAc). Saturated macrocycle 25a (0.12 g) was obtained in 81% yield as a colorless oil: HPLC t_{R}=7.44 min. ¹H-NMR (CDCl₃, 400 MHz) δ 9.17 (s, 1H), 8.47 (d, J=7.5 Hz, 1H), 7.15-7.06 (m, 3H), 4.26 (dt, J=11.2, 2.2 Hz 1H), 4.05 (dt, J=11.5, 3.7 Hz, 1H), 3.77 (dd, J=8.4, 4.1 Hz, 1H), 3.68 (s, 3H), 3.61 (m, 1H), 3.54 (m, 1H), 2.33 (t, J=7.5 Hz, 1H), 2.28 (m, 1H), 2.00 (m, 1H), 1.86 (m, 1H), 1.78-1.34 (m, 12H). ¹³C-NMR (CDCl₃, 100 MHz) δ 173.8, 171.4, 148.4, 130.8, 124.0, 123.7, 120.3, 119.2, 81.6, 75.4, 67.3, 51.1, 33.6, 33.2, 28.5, 27.2, 26.1, 24.9, 24.4, 19.7. MS (ESI) m/z: 364.1 (M+1).

Macrocycle 25b was prepared according to the procedure described above in 99% yield. A colorless oil: ¹H-NMR (CDCl₃, 400 MHz) δ 9.25 (s, 1H), 8.39 (dd, J=7.8, 1.7 Hz, 1H), 7.05 (m, 2H), 6.99 (dt, J=7.7, 1.7 Hz, 1H), 4.39 (ddd, J=9.7, 4.9 Hz, 1H), 3.96 (dt, J=9.3, 4.2 Hz, 1H), 3.74 (dd, J=8.2, 4.0 Hz, 1H), 3.67 (s, 3H), 3.61, (m, 2H), 2.33 (t, J=7.5 Hz, 2H), 1.95-1.74 (m, 6H), 1.72-1.61 (m, 3H), 1.58-1.48 (m, 5H), 1.42-1.36 (m, 2H). ¹³C-NMR (CDCl₃, 100 MHz) δ 173.8, 171.0, 147.1, 127.9, 123.4, 121.4, 119.2, 112.9, 81.4, 69.8, 68.5, 51.1, 33.7, 33.1, 28.6, 28.2, 26.4, 24.8, 24.7, 24.4, 23.4. MS (ESI) m/z: 378.2 (M+1).

Macrocycle 25c was prepared according to the procedure described above in 90% yield. A colorless oil: ¹H-NMR (CDCl₃, 400 MHz) δ 9.10 (s, 1H), 8.56 (dd, J=8.0, 1.6 Hz, 1H), 7.05 (dt, J=7.8, 1.6 Hz, 1H), 6.97 (dt, J=7.9, 1.2 Hz, 1H), 6.87 (dd, J=8.1, 1.2 Hz, 1H), 4.22 (m, 1H), 3.97 (t, J=9.3 Hz, 1H), 3.78 (dd, J=7.8, 4.0 Hz, 1H), 3.68 (s, 3H), 3.67 (m, 1H), 3.52 (dt, J=8.9, 3.8 Hz, 1H), 2.33 (t, J=7.5 Hz, 2H), 2.11-1.78 (m, 3H), 1.76-1.62 (m, 8H), 1.57-1.46 (m, 3H), 1.45-1.30 (m, 4H). ¹³C-NMR (CDCl₃, 100 MHz) δ 173.8, 171.0, 147.3, 126.6, 123.3, 120.4, 118.8, 109.7, 81.4, 70.6, 69.5, 51.1, 33.7, 33.0, 29.4, 29.0, 28.6, 26.7, 25.3, 24.6, 24.5, 23.9. MS (ESI) m/z: 392.2 (M+1).

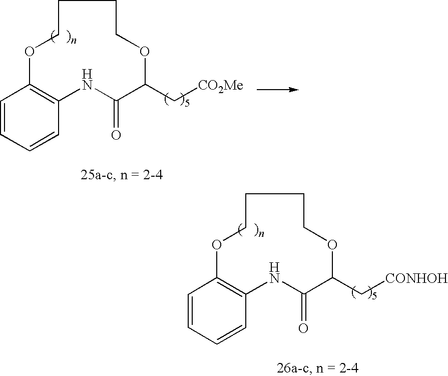

(±)-6-(6-Oxo-6,7,9,10,11,12-hexahydro-5H-8,13-dioxa-5-azabenzocycloundecen-7-yl)hexanoic acid hydroxyamide (26a), (±)-6-(6-Oxo-6,7,10,11,12,13-hexahydro-5H,9H-8,14-dioxa-5-azabenzocyclododecen-7-yl)hexanoic acid hydroxyamide (26b), (±)-6-(6-Oxo-6,7,9,10,11,12,13,14-octahydro-5H-8,15-dioxa-5-azabenzocyclotridecen-7-yl)hexanoic acid hydroxyamide (26c). Hydroxamic acid 26a was prepared according to the general procedure 7A starting from the corresponding methyl ester 25a in 99% yield. A colorless oil: HPLC t_{R}=5.49 min. ¹H-NMR (δ₆-DMSO, 400 MHz) δ 10.34 (s, 1H), 9.13 (s, 1H), 8.68 (s, 1H), 8.33 (s, 1H), 8.27 (m, 1H), 7.22 (m, 1H), 7.08 (m, 2H), 4.18 (t, J=9.3 Hz, 1H), 4.03 (m, 2H), 4.76 (dd, J=8.1, 4.2 Hz, 1H), 3.59 (m, 1H), 3.47 (t, J=9.4 Hz, 1H), 2.08 (m, 1H), 1.94 (t, J=7.3 Hz, 2H), 1.83 (m, 1H), 1.70 (m, 1H), 1.62 (m, 2H), 1.51-1.46 (m, 3H), 1.37 (m, 2H), 1.26 (m, 2H). ¹³C-NMR (δ₆-DMSO, 100 MHz) δ 171.3, 169.1, 148.7, 130.9, 124.5, 123.6, 121.0, 119.0, 81.0, 75.2, 67.5, 33.0, 32.3, 28.4, 27.1, 26.0, 25.1, 24.8, 19.8. HRMS (ES+) C₁₉H₂₈N₂O₅ calcd for [MH]⁺ 365.20710. found 365.20708.

Hydroxamic acid 26b was prepared according to the general procedure 7A starting from the corresponding methyl ester 25b in 99% yield. A colorless oil: HPLC t_{R}=6.02 min. ¹H-NMR (δ₆-DMSO, 400 MHz) δ 10.35 (s, 1H), 9.21 (s, 1H), 8.68 (s, 1H), 8.23 (dd, J=7.9, 1.4 Hz, 1H), 7.16 (dd, J=8.1, 1.0 Hz, 1H), 7.06 (dt, J=7.5, 1.5 Hz, 1H), 6.97 (dt, J=7.7, 1.1 Hz, 1H), 4.37 (ddd, J=9.9, 4.8 Hz, 1H), 3.97 (dt, J=9.1, 4.0 Hz, 1H), 3.76 (dd, J=8.0, 4.1 Hz, 1H), 3.62 (m, 1H), 3.51 (t, J=9.4 Hz, 1H), 1.94 (t, J=7.1 Hz, 2H), 1.80-1.56 (m, 6H), 1.52-1.23 (m, 10H). ¹³C-NMR (δ₆-DMSO, 100 MHz) δ 170.8, 169.2, 147.2, 128.0, 123.9, 121.5, 118.6, 114.1, 80.7, 70.0, 68.8, 32.8, 32.3, 28.4, 28.2, 26.2, 25.1, 24.7 (2C), 23.5. HRMS (ES+) C₂₀H₃₀N₂O₅ calcd for [MH]⁺ 379.22275. found 379.22294.

Hydroxamic acid 26c was prepared according to the general procedure 7A starting from the corresponding methyl ester 25c in 99% yield. A colorless oil: HPLC t_{R}=6.54 min. ¹H-NMR (δ₆-DMSO, 400 MHz) δ 10.36 (s, 1H), 9.04 (s, 1H), 8.67 (s, 1H), 8.37 (d, J=7.8 Hz, 1H), 7.04 (m, 2H), 6.92 (t, J=7.1 Hz, 1H), 4.20 (m, 1H), 3.97 (t, J=9.8 Hz, 1H), 3.82 (dd, J=7.4, 4.1 Hz, 1H), 3.58 (m, 2H), 1.93 (t, J=7.1 Hz, 2H), 1.92-1.70 (m, 6H), 1.69-1.58 (m, 3H), 1.55-1.43 (m, 3H), 1.40-1.20 (m, 6H). ¹³C-NMR (δ₆-DMSO, 100 MHz) δ 170.6 (2C), 147.4, 126.6, 123.9, 120.4, 118.2, 111.1, 80.6, 70.9, 69.4, 32.6, 32.3, 29.3, 28.7, 28.5, 27.0, 25.3, 25.1, 24.4, 24.1. HRMS (ES+) C₂₁H₃₂N₂O₅ calcd for [MH]⁺ 393.23840. found 393.23842.

### Example 8

### Preparation of Macrocyclic Hydroxamic Acids Embedding a Second Aromatic Ring

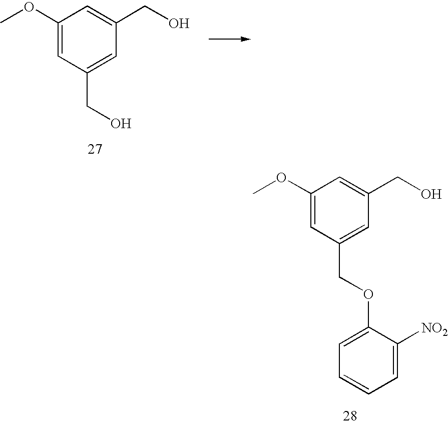

[3-Methoxy-5-(2-nitrophenoxymethyl)phenyl]methanol (28). Nitrophenoxy derivative 28 was prepared according to the general procedure (Method 2C2) starting from alcohol 27 (Zimmerman, H. E.; Jones II, G. J. Am. Chem. Soc. 1970, 92, 2753-2761) (1.23 g, 7.31 mmol), o-nitrophenol (1.22 g, 8.78 mmol), Ph₃P (2.36 g, 8.78 mmol) and DIAD (1.73 mL, 8.78 mmol) in anhydrous THF (94.0 mL). After purification by flash chromatography (gradient 1:1 to 6:4 EtOAc/hexanes) nitrophenoxy compound 28 (1.29 g) was isolated in 61% yield as a yellow solid: ¹H-NMR (CDCl₃, 400 MHz) δ 7.88 (dd, J=8.1, 1.6 Hz, 1H), 7.52 (dt, J=7.8, 1.7 Hz, 1H), 7.13 (d, J=7.9 Hz, 1H), 7.06 (dt, J=7.7, 1.0 Hz, 1H), 7.03 (s, 1H), 7.00 (s, 1H), 6.90 (s, 1H), 5.23 (s, 2H), 4.70 (d, J=4.7 Hz, 2H), 3.85 (s, 3H), 1.87 (t, J=5.1 Hz, 1H). ¹³C-NMR (CDCl₃, 100 MHz) δ 159.9 (2C), 151.5, 142.6, 137.1, 133.8, 125.4, 120.4, 116.9, 114.7, 111.9, 111.1, 70.5, 64.7, 55.0.

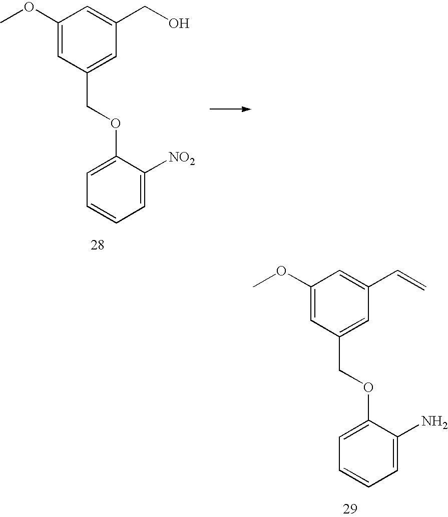

2-(3-Methoxy-5-vinylbenzyloxy)phenylamine (29). A solution of benzyl alcohol 28 (1.20 g, 4.15 mmol) and pyridinium dichromate (PDC, 2.34 g, 6.23 mmol) in CH₂Cl₂ was stirred at room temperature under an argon atmosphere. After 18 h the reaction mixture was filtered through a pad of silica gel/Celite® washing with EtOAc. The filtrate was concentrated in vacuo and purified by flash chromatography (gradient 1:1 to 6:4 EtOAc/hexanes) to afford the pure aldehyde intermediate (1.09 g) in 91% yield as a pale yellow solid: ¹H-NMR (CDCl₃, 400 MHz) δ 10.0 (s, 1H), 7.91 (dd, J=8.1, 1.6 Hz, 1H), 7.55 (dt, J=7.8, 1.7 Hz, 1H), 7.37 (m, 2H), 7.15 (s, 1H), 7.13 (s, 1H); 7.10 (dt, J=7.7, 1.0 Hz, 1H), 5.29 (s, 2H), 3.91 (s, 3H). ¹³C-NMR (CDCl₃, 100 MHz) δ 191.5, 160.2 (2C), 151.2, 137.9, 137.7, 133.9, 125.5, 120.7 (2C), 118.7, 114.6, 112.5, 69.7, 55.3.

To a solution of methyltriphenylphosphonium bromide (2.03 g, 5.67 mmol) in anhydrous THF (27.0 mL) under an argon, NaHMDS (1.0 M solution in THF, 5.29 mL) was added dropwise at 0° C. After stirring at the same temperature for 15 min a solution of the above aldehyde intermediate in THF (20.0 mL) was slowly added and the yellow mixture was allowed to react at room temperature. After 3 h the reaction was judged complete, quenched with H₂O, and extracted with CH₂Cl₂. The organic phase was dried (MgSO₄), concentrated in vacuo, and subjected to flash chromatographic purification (7:3 hexanes/EtOAc). Pure olefin intermediate (1.60 g) was obtained in 99% yield as an orange oil: ¹H-NMR (CDCl₃, 400 MHz) δ 7.88 (dd, J=8.1, 1.7 Hz, 1H), 7.52 (dt, J=7.9, 1.7 Hz, 1H), 7.13 (dd, J=8.0, 1.0 Hz, 1H), 7.10 (s, 1H), 7.06 (dt, J=7.81.1 Hz, 1H), 6.99 (s, 1H), 6.92 (s, 1H), 6.70 (dd, J=17.6, 10.9 Hz, 1H), 5.79 (d, J=17.3 Hz, 1H), 5.30 (d, J=11.0 Hz, 1H), 5.22 (s, 2H), 3.86 (s, 3H). ¹³C-NMR (CDCl₃, 100 MHz) δ 159.8 (2C), 151.5, 139.0, 137.0, 136.0, 133.8, 125.4, 120.3, 116.7, 114.7, 114.4, 111.3, 111.2, 70.4, 55.0.

This nitrobenzene intermediate was reduced to aniline derivative 29 following the general procedure 3A1 (aqueous work-up and extraction) or according to Method 3A2. After flash chromatography (CH₂Cl₂100%) pure aniline 29 (0.78 g) was isolated in 87% yield as an orange oil: ¹H-NMR (CDCl₃, 400 MHz) δ 7.11 (s, 1H), 6.95 (m, 2H), 6.90-6.83 (m, 2H), 6.79-6.74 (m, 2H), 6.74 (dd, J=17.2, 11.1 Hz, 1H), 5.80 (d, J=17.4 Hz, 1H), 5.32 (d, J=11.2 Hz, 1H), 5.07 (s, 2H), 3.81 (s, 3H), 3.65 (b, 2H). ¹³C-NMR (CDCl₃, 100 MHz) δ 159.7 (2C), 146.1, 138.9, 138.6, 136.2, 121.2, 118.1, 117.7, 114.9, 114.3, 112.3, 111.8, 110.6, 69.9, 55.0. MS (ESI) m/z: 256.1 (M+1).

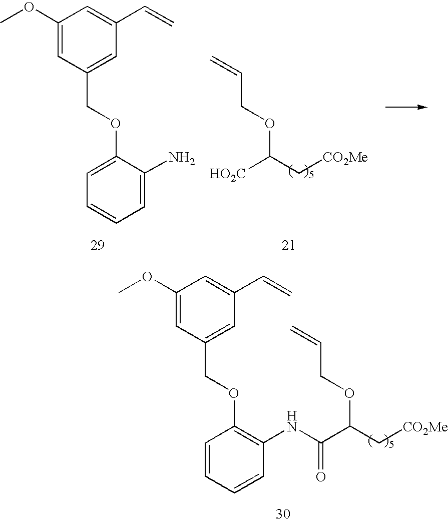

(±)-7-Allyloxy-7-[2-(3-methoxy-5-vinylbenzyloxy)phenylcarbamoyl]heptanoic acid methyl ester (30). Anilide 31 was obtained following the general procedure (Method 1B) starting from carboxylic acid 21 (0.25 g, 1.02 mmol), aniline 29 (0.39 g, 1.53 mmol), EDC (0.68 g, 3.57 mmol), HOBt (0.18 g, 1.35 mmol) and DIPEA (0.62 mL, 3.57 mmol) in anhydrous CH₂Cl₂ (5.0 mL). After flash chromatographic purification (gradient 8:2 to 75:25 hexanes/EtOAc) anilide 30 was isolated in 60% yield as a pale yellow oil: ¹H-NMR (CDCl₃, 400 MHz) δ 9.15 (s, 1H), 8.44 (dd, J=7.8, 1.7 Hz, 1H); 7.08-6.89 (m, 6H); 6.71 (dd, J=17.6, 10.9 Hz, 1H); 5.80 (d, J=17.7 Hz, 1H), 5.75 (ddt, J=17.1, 10.4, 5.6 Hz, 1H); 5.31 (d, J=10.5 Hz, 1H), 5.20 (dd, J=17.2, 1.5 Hz, 1H), 5.10 (s, 2H), 5.08 (dd, J=10.4, 1.3 Hz, 1H), 4.02 (ddd, J=24.1, 12.6, 5.4 Hz, 2H), 3.88 (dd, J=7.2, 4.3 Hz, 1H), 3.85 (s, 3H), 3.67 (s, 3H), 2.31 (t, J=7.5 Hz, 2H), 1.82 (m, 2H), 1.64 (m, 2H), 1.47 (m, 2H), 1.35 (m, 2H). ¹³C-NMR (CDCl₃, 100 MHz) δ 173.8, 170.6, 159.7, 147.0, 139.0, 137.7, 136.1, 133.1, 127.0, 123.6, 121.1, 119.5, 117.6 (2C), 114.4, 112.2, 111.1, 110.8, 79.8, 71.4, 70.1, 54.9, 51.1, 33.6, 32.5, 28.6, 24.4, 24.3. MS (ESI) m/z: 482.3 (M+1).

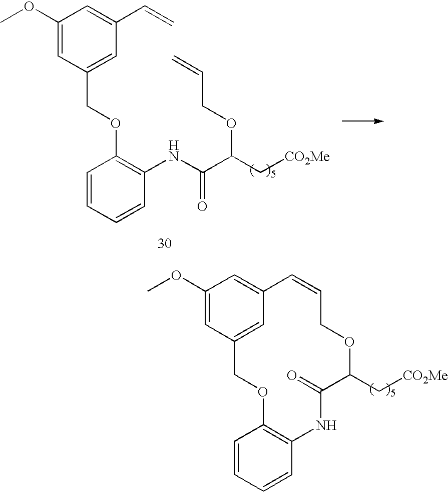

(±)-6-(19-Methoxy-11-oxo-3,13-dioxa-10-azatricyclo[15.3.1.04,9]henicosa-1(21),4(9),5,7,15,17,19-heptaen-12-yl)hexanoic acid methyl ester (31).

Macrocyclic olefin 31 was prepared according to the general procedure 5A starting from the corresponding diolefin precursor 30. After purification by flash chromatography (gradient 8:2 to 1:1 hexanes/EtOAc) pure macrocycle 31 was obtained in 52% yield (83%, two cycles) as a sole cis isomer. A pale yellow oil: HPLC t_{R}=7.92 min. ¹H-NMR (CDCl₃, 400 MHz) δ 9.28 (s, 1H), 8.38 (d, J=7.6 Hz, 1H), 7.64 (s, 1H), 7.10 (m, 2H), 7.03 (m, 1H), 6.87 (d, J=11.2 Hz, 1H), 6.67 (s, 2H), 6.05 (ddd, J=10.6, 6.9 Hz, 1H), 5.40 (d, J=12.7 Hz, 1H), 5.06 (d, J=12.8 Hz, 1H), 4.50 (t, J=10.0 Hz, 1H), 3.87 (dd, J=8.4, 4.1 Hz, 1H), 3.81 (s, 3H), 3.78 (m, 1H), 3.69 (s, 3H), 2.35 (t, J=7.4 Hz, 2H), 1.92 (m, 1H), 1.85 (m, 1H), 1.73-1.51 (m, 4H), 1.42 (m, 2H). ¹³C-NMR (CDCl₃, 100 MHz) δ 173.8, 170.5, 159.3, 147.2, 138.5, 137.3, 135.9, 128.2, 125.6, 123.7, 121.9, 119.5, 118.4, 113.7, 112.3, 111.0, 83.9, 70.9, 68.4, 55.0, 51.2, 33.7 (2C), 28.5 25.0, 24.5. MS (ESI) m/z: 454.2 (M+1).

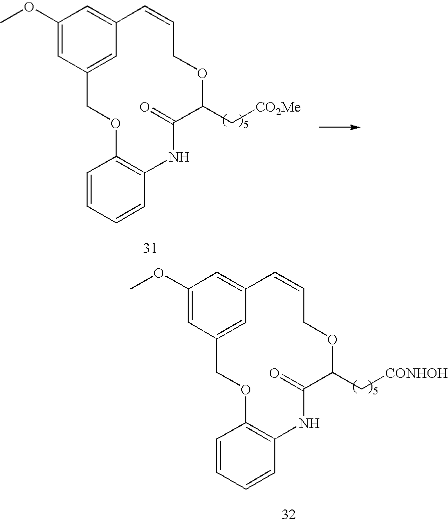

(±)-6-(19-Methoxy-11-oxo-3,13-dioxa-10-azatricyclo[15.3.1.04,9]henicosa-1(21),4(9),5,7,15,17,19-heptaen-12-yl)hexanoic acid hydroxyamide (32). Hydroxamic acid 32 was prepared according to the general procedure 7A starting from the corresponding methyl ester 31 in 99% yield. A colorless oil: HPLC t_{R}=6.08 min. ¹H-NMR (δ₆-DMSO, 400 MHz) δ 10.37 (s, 1H), 9.34 (s, 1H), 8.69 (s, 1H), 8.00 (d, J=7.9 Hz, 1H), 7.56 (s, 1H), 7.27 (d, J=8.1 Hz, 1H), 7.15 (t, J=7.7 Hz, 1H), 6.99 (t, J=7.7 Hz, 1H), 6.83 (s, 2H), 6.79 (d, J=12.8 Hz, 1H), 6.00 (ddd, J=10.4, 7.2 Hz, 1H), 5.32 (d, J=13.0 Hz, 1H), 5.19 (d, J=13.0 Hz, 1H), 4.32 (t, J=9.9 Hz, 1H), 3.90 (m, 2H), 3.75 (s, 3H), 1.96 (t, J=7.3 Hz, 2H), 1.78 (m, 1H), 1.71 (m, 1H), 1.56-1.40 (m, 4H), 1.32 (m, 2H). ¹³C-NMR (δ₆-DMSO, 100 MHz) δ 170.5, 169.2, 159.4, 148.5, 139.2, 137.3, 135.4, 128.0, 126.5, 124.8, 121.7, 120.5, 118.5, 114.7, 112.9, 111.6, 82.5, 70.4, 67.6, 55.3, 33.1, 32.3, 28.5, 25.2, 24.7. HRMS (ES+) C₂₅H₃₀N₂O₆ calcd for [MH]⁺ 454.21766. found 455.21716.

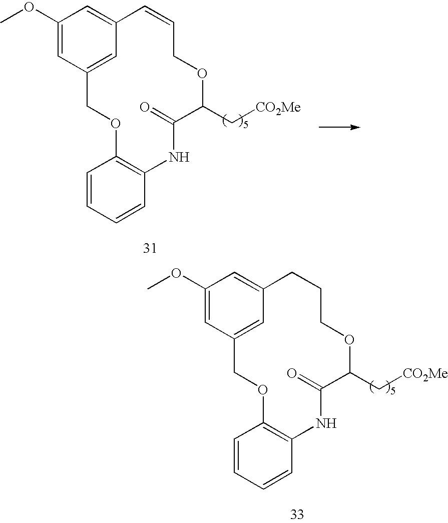

(±)-6-(19-Methoxy-11-oxo-3,13-dioxa-10-azatricyclo[15.3.1.04,9]henicosa-1(21),4(9),5,7,17,19-hexaen-12-yl)hexanoic acid methyl ester (33). Macrocyclic olefin 31 (0.20 g, 0.42 mmol) was dissolved in EtOAc (4.0 mL), then n-butylamine (for the use of this additive for preventing 0-benzyl cleavage, see Czech, B. P.; Bartsch, R. A. J. Org. Chem. 1984, 49, 4076-4078.) (41 λL, 0.79 mmol) and catalytic 5% palladium on carbon (0.1 mg/mmol) were added. The reaction vessel was evacuated by aspiration and thoroughly purged with H₂ (three times), and the resulting heterogeneous mixture was stirred under a balloon of H₂. After 4 h the H₂ was evacuated, the catalyst filtered off, and the filtrate concentrated under reduced pressure to give a crude which was subjected to flash chromatography (7:3 hexanes/EtOAc). Saturated macrocycle 33 (0.20 g) was obtained in 99% yield as a colorless oil: HPLC t_{R}=7.88 min. ¹H-NMR (CDCl₃, 400 MHz) δ 8.49 (s, 1H), 8.29 (dd, J=8.0, 1.4 Hz, 1H), 7.35 (s, 1H), 7.17 (dd, J=8.0, 1.3 Hz, 1H), 7.08 (ddd, J=7.5, 1.5 Hz, 1H), 7.02 (t, J=8.0 Hz, 1H), 6.66 (s, 1H), 6.40 (s, 1H), 5.31 (d, J=11.5 Hz, 1H), 4.94 (d, J=12.1 Hz, 1H), 3.66 (t, J=5.8 Hz, 1H), 3.71 (s, 3H), 3.68 (s, 3H), 3.35 (m, 1H), 3.81 (m, 1H), 2.38 (t, J=7.5 Hz, 2H), 2.18 (m, 1H), 1.81 (m, 3H), 1.66 (m, 3H), 1.47-1.32 (m, 5H). ¹³C-NMR (CDCl₃, 100 MHz) δ 173.8, 170.7, 159.2, 147.6, 143.1, 138.1, 129.6, 123.9, 122.8, 121.1, 120.0, 117.7, 113.1, 110.7, 82.4, 75.2, 68.3, 54.8, 51.1, 33.6, 32.2, 31.8, 29.4, 28.6, 24.5, 24.1. MS (ESI) m/z: 456.1 (M+1).

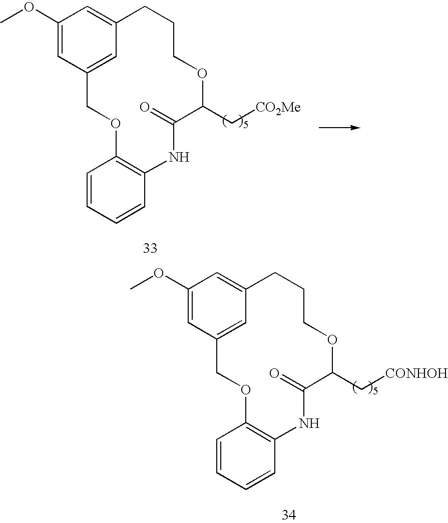

(±)-6-(19-Methoxy-11-oxo-3,13-dioxa-10-azatricyclo[15.3.1.04,9]henicosa-1(21),4(9),5,7,17,19-hexaen-12-yl)hexanoic acid hydroxyamide (34). Hydroxamic acid 34 was prepared according to the general procedure 7A starting from the corresponding methyl ester 33 in 99% yield. A colorless oil: HPLC t_{R}=6.08 min. ¹H-NMR (δ₆-DMSO, 400 MHz) δ 10.35 (s, 1H), 8.78 (s, 1H), 8.62 (s, 1H), 7.96 (dd, J=8.0, 1.2 Hz, 1H), 7.32 (d, J=8.1 Hz, 1H), 7.13 (dt, J=7.4, 1.4 Hz, 1H), 7.98 (t, J=7.5 Hz, 1H), 6.72 (s, 1H), 6.51 (s, 1H), 5.20 (d, J=12.0 Hz, 1H), 5.06 (d, J=12.1 Hz, 1H), 4.12 (dd, J=10.5, 5.2 Hz, 1H), 3.81 (t, J=5.8 Hz, 1H), 3.65 (s, 3H), 3.40-3.35 (m, 1H), 3.25 (m, 1H), 2.75 (m, 2H), 2.03 (m, 1H), 1.93 (t, J=7.3 Hz, 2H), 1.82 (m, 1H), 11.67 (m, 2H), 1.45 (m, 2H), 1.20-1.29 (m, 4H). ¹³C-NMR (δ₆-DMSO, 100 MHz) δ 170.4, 169.1, 159.1, 148.6, 143.1, 138.5, 129.2, 124.8, 122.5, 121.3, 120.7, 118.1, 113.2, 111.3, 81.0, 74.0, 67.9, 55.0, 32.3, 31.9, 31.5, 29.1, 28.5, 25.1, 24.0. HRMS (ES+) C₂₅H₃₂N₂O₆ calcd for [MH]⁺ 451.23331. found 451.23297. Mol. Wt.: 456.53.

### Example 9

### Preparation of Macrocyclic Hydroxamic Acids Embedding an Amino Group in the Aliphatic Tether

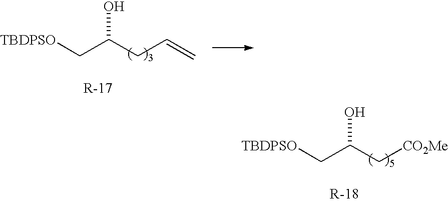

8-(tert-Butyldiphenylsilanyloxy)-7-(R)-hydroxyoctanoic acid methyl ester [(R)-18]. Enantiopure (R)-18 was prepared from olefin (R)-17 following the same procedure described for the enantiomer (S)-18. A colorless oil: ¹H- and ¹³C-NMR analyses were consistent to those reported for (S)-18.

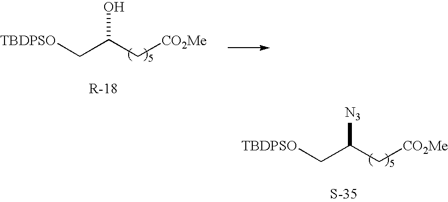

7-(S)-Azido-8-(tert-butyldiphenylsilanyloxy)octanoic acid methyl ester [(S)-35]. To a solution of DEAD (0.64 mL, 40% wt in toluene, 1.40 mmol) and DPPA (0.39 mL, 1.40 mmol), alcohol (R)-18 (400 mg, 1.07 mmol) in anhydrous toluene (3.7 mL) was added dropwise at 0° C. under argon atmosphere, followed by PPh₃ (0.37 g, 1.40 mmol). The reaction was allowed to warm slowly to rt, and stirred for 24 h. The solvent was removed under vacuum, and the crude residue was purified by flash chromatography (9:1 hexanes/EtOAc) to obtain azide (S)-35 (0.36 g, 85% yield) as a colorless oil: [α]²⁰_{D} −12.9 (c 1.3, CHCl₃). ¹H-NMR (CDCl₃, 400 MHz) δ 7.72-7.69 (m, 4H), 7.47-7.41 (m, 6H), 3.73 (dd, J=10.6, 3.9 Hz, 1H), 3.69 (s, 3H), 3.65 (dd, J=10.6, 6.8 Hz, 1H), 3.42-3.36 (m, 1H), 2.31 (t, J=7.4 Hz, 2H), 1.70-1.58 (m, 2H), 1.48-1.41 (m, 3H), 1.34-1.26 (m, 3H), 1.11 (s, 9H). ¹³C-NMR (CDCl₃, 100 MHz) δ 173.7, 135.2 (4C), 132.7 (2C), 129.4 (2C), 127.4 (4C), 66.6, 63.4, 51.1, 33.5, 29.8, 28.5, 26.4 (3C), 25.3, 24.3, 18.8. MS (ESI) m/z: 454.2 (M+1).

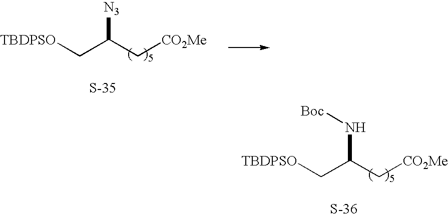

7-(S)-tert-Butoxycarbonylamino-8-(tert-butyldiphenylsilanyloxy)octanoic acid methyl ester [(S)-36]. Azide (S)-35 (0.35 g, 0.77 mmol) was dissolved in MeOH (7.7 mL), and catalytic 10% palladium on carbon (0.1 mg/mmol) was added. The reaction vessel was evacuated by aspiration and thoroughly purged with H₂ (three times), and the resulting heterogeneous mixture was stirred under a balloon of H₂. After 18 h the H₂ was evacuated, the catalyst filtered off, and the filtrate concentrated under reduced pressure to give crude amine intermediate (0.33 g, 99% yield) which was subjected to the next reaction without any purification. For analytical purpose, pure amine intermediate was isolated after flash chromatography (100% EtOAc). A colorless oil: ¹H-NMR (CDCl₃, 400 MHz) δ 7.70-7.67 (m, 4H), 7.45-7.39 (m, 6H), 3.67 (s, 3H), 3.62 (dd, J=9.9, 4.1 Hz, 1H), 3.43 (dd, J=9.9, 7.2 Hz, 1H), 2.89-2.82 (m, 1H), 2.30 (t, J=7.5 Hz, 2H), 1.64-1.59 (m, 2H), 1.43-1.36 (m, 4H), 1.35-1.26 (m, 4H), 1.09 (s, 9H). ¹³C-NMR (CDCl₃, 100 MHz) δ 173.8, 135.2 (4C), 132.3 (2C), 129.3 (2C), 127.3 (4C), 68.7, 52.6, 51.0, 33.6, 33.2, 28.9, 26.5 (3C), 25.4, 24.5, 18.9. MS (ESI) m/z: 428.3 (M+1).

To a solution of this crude amine (0.33 g, 0.77 mmol) in anhydrous CH₂Cl₂, Boc₂O (0.20 g, 0.92 mmol) was added in one portion, and the reaction was left under stirring for 24 h. The solvent was removed in vacuo, and the crude purified by flash chromatography (9:1 hexanes/EtOAc) which afforded pure N-Boc protected amine (S)-36 (0.37 g, 90% yield) as a colorless oil: [α]²⁰_{D} −8.5 (c 1.1, CHCl₃). ¹H-NMR (CDCl₃, 400 MHz) δ 7.67-7.65 (m, 4H), 7.45-7.40 (m, 6H), 4.65 (bs, 1H), 3.73-3.58 (m, 3H), 3.69 (s, 3H), 2.31 (t, J=7.5 Hz, 2H), 1.65-1.58 (m, 2H), 1.49-1.44 (m, 3H), 1.47 (s, 9H), 1.34-1.24 (m, 3H), 1.10 (s, 9H). ¹³C-NMR (CDCl₃, 100 MHz) δ 173.8, 155.2, 135.2 (4C), 133.0 (2C), 129.4, 129.3, 127.3 (4C), 65.3 (2C), 51.0, 33.6, 31.4, 28.7, 28.1 (4C), 26.6 (3C), 25.3, 24.5, 18.6. (MS (ESI) m/z: 528.4 (M+1).

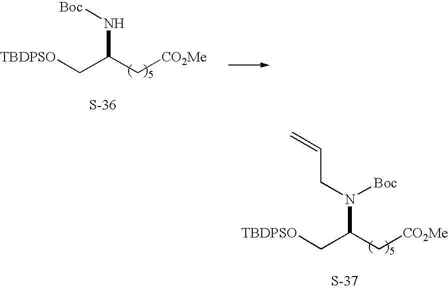

7-(S)-Allyl-tert-butoxycarbonylamino)-8-(tert-butyldiphenylsilanyloxy)octanoic acid methyl ester [(S)-37]. Protected amine (S)-36 (0.36 g, 0.68 mmol) was dissolved in anhydrous DMF (5.0 mL) under argon, and the solution was cooled on an ice-bath. NaH (82 mg, 60% wt dispersion in mineral oil, 2.05 mmol) was carefully added portionwise while stirring, immediately followed by allyliodide (0.37 mL, 4.09 mmol). The reaction was stirred at the same temperature for 1 h, after which time NH₄Cl (aq, sat.) was cautiously added. After stirring 10 min at rt, the mixture was extracted with EtOAc, the organic phase was washed with brine, dried (MgSO₄), and concentrated under vacuum. Flash chromatographic purification (9:1 hexanes/EtOAc) afforded N-Boc-allylamine (S)-37 (0.35 g, 89% yield) as a colorless oil: [α]²⁰_{D} −6.4 (c 1.0, CHCl₃). ¹H-NMR (CDCl₃, 400 MHz), mixture of atropoisomers, δ 7.68-7.67 (m, 4H), 7.47-7.38 (m, 6H), 5.99-5.80 (m, 1H), 5.19-5.00 (m, 2H), 4.20-4.10 (m, ½H), 3.99-3.55 (m, 4½H), 3.68 (s, 3H), 2.30 (bt, 2H), 1.65-1.60 (m, 2H), 1.48-1.40 (m, 12H), 1.32-1.23 (m, 3H), 1.08 (s, 9H). ¹³C-NMR (CDCl₃, 100 MHz), mixture of atropoisomers, δ 173.7, 155.4, 136.2+135.6 (1C), 135.2 (4C), 133.2 (2C), 129.3 (2C), 127.3 (4C), 115.4+114.8 (1C), 78.8, 64.7+64.4 (1C), 57.8+56.9 (1C), 51.0, 33.6, 28.6, 28.1 (4C), 26.7, 26.4 (3C), 25.6, 24.4, 18.9. MS (ESI) m/z: 568.4 (M+1).

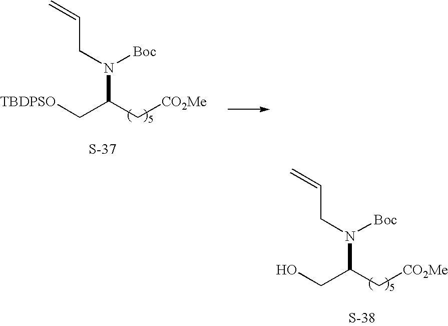

7-(S)-Allyl-tert-butoxycarbonylamino)-8-hydroxyoctanoic acid methyl ester [(S)-38]. A solution of fully protected amino alcohol (5) 37 (0.34 g, 0.60 mmol) in THF (6.0 mL) was treated with a solution of TBAF/AcOH (1:1, ca 1M in THF, 0.90 mL, 0.90 mmol) at 0° C. under argon. After warming to room temperature the reaction mixture was stirred at room temperature and monitored by TLC until complete conversion of the starting material. After 24 h the reaction was judged complete, quenched with NH₄Cl (aq, sat.) and extracted with CH₂Cl₂. The organic phase was dried (MgSO₄), concentrated under reduced pressure and purified by flash chromatography (1:1 hexanes/EtOAc), which furnished 0.20 g of alcohol (S)-38 as a colorless oil (97% yield): [α]²⁰_{D} −1.5 (c 0.4, CHCl₃). ¹H-NMR (CDCl₃, 400 MHz), mixture of atropoisomers, δ 5.90-5.78 (m, 1H), 5.19-5.10 (m, 2H), 3.80-3.59 (m, 5H), 3.69 (s, 3H), 2.82 (bs, 1H), 2.30 (t, J=7.4 Hz, 2H), 1.64-1.58 (m, 2H), 1.5-1.52 (m, 2H), 1.46 (s, 9H), 1.39-1.26 (m, 4H). ¹³C-NMR (CDCl₃, 100 MHz), mixture of atropoisomers, δ 173.7, 135.2 (2C), 115.9, 79.7, 58.6, 51.0, 33.6 (2C), 28.6 (2C), 28.0 (3C), 25.6, 24.4 (2C). MS (ESI) m/z: 330.3 (M+1).

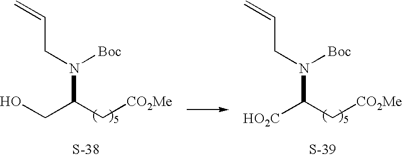

2-(S)-(Allyl-tert-butoxycarbonylamino)octanedioic acid 8-methyl ester [(S)-39]. To a solution of oxalyl chloride (0.87 mL, 2.0 M solution in CH₂Cl₂, 1.74 mmol) at −78° C. under an argon atmosphere, DMSO (0.17 mL, 2.32 mmol) in CH₂Cl₂ (11.5 mL) was added dropwise. After 10 min a solution of alcohol (S)-38 (0.19 g, 0.58 mmol) in CH₂Cl₂ (3.3 mL) was added at the same temperature. After 1 h Et₃N (0.80 mL, 5.77 mmol) was added, stirring was continued at −78° C. for 30 min, then the reaction was allowed to reach room temperature over a period of 1 h. NH₄Cl (aq, sat.) was added, and the mixture was extracted with CH₂Cl₂. The organic phase was dried (MgSO₄), concentrated in vacuo and the resulting residue purified by flash chromatography (8:2 hexanes/EtOAc) which furnished the pure aldehyde intermediate (0.18 g) in 93% yield as a colorless oil: [α]²⁰_{D} −56.2 (c 0.7, CHCl₃). ¹H-NMR (CDCl₃, 400 MHz), mixture of atropoisomers, δ 9.56 (d, J=12.4 Hz, 1H), 5.80-5.75 (m, 1H), 5.25-5.15 (m, 2H), 4.35-4.25 (bdd, ½H), 4.05-3.90 (m, 1H), 3.68 (s, 3H), 3.62 (dd, J=15.5, 6.9 Hz, 1H), 3.58-3.51 (bdd, ½H), 2.32 (t, J=7.3 Hz, 2H), 2.05-1.94 (m, 1H), 1.72-1.62 (m, 3H), 1.48-1.33 (m, 13H). ¹³C-NMR (CDCl₃, 100 MHz), mixture of atropoisomers, δ 199.8+199.4 (1C), 173.7, 155.0+154.9 (1C), 134.0+133.6 (1C), 118.1+117.0 (1C), 81.1+80.1 (1C), 65.3, 51.2, 50.7, 49.7, 33.5 (2C), 27.8 (3C), 24.3 (2C). MS (ESI) m/z: 328.2 (M+1).

A solution of this aldehyde intermediate in acetone (5.5 mL) was treated at 0° C. with Jones reagent (0.73 mL, prepared from 27.0 g of chromium (VI) oxide, 23.0 mL of H₂SO₄, and 75.0 mL of water). After 15 min at 0° C. the reaction was quenched with ⁱPrOH (27.0 mL), and partitioned between EtOAc and water. The organic layer was washed with 10% NaHSO₄, 10% Na₂S₂O₃, water, dried (MgSO₄) and concentrated in vacuo to afford the carboxylic acid 21 as an amorphous white solid (0.20 g, 99% yield) which was used for the next reaction without further purification: [α]²⁰_{D} −23.0 (c 0.9, CHCl₃). ¹H-NMR (CDCl₃, 400 MHz), mixture of atropoisomers, δ 9.00 (b, 1H), 6.00-5.80 (m, 1H), 5.30-5.11 (m, 2H), 4.39-4.36 (m, ½H), 4.15-4.11 (m, ½H), 4.00-3.91 (m, 1H), 3.78-3.61 (m, 1H), 3.68 (s, 3H), 2.32 (t, J=7.5 Hz, 2H), 2.03-1.95 (m, 1H), 1.83-1.76 (m, 1H), 1.65-1.60 (m, 2H), 1.47-1.32 (m, 13H). ¹³C-NMR (CDCl₃, 100 MHz), mixture of atropoisomers, δ 177.0+176.1 (1C), 173.8, 155.9+149.5 (1C), 134.3, 117.2+116.4 (1C), 80.6, 68.2, 59.2+58.7 (1C), 51.2, 50.1+49.3 (1C), 33.6 (2C), 27.9 (3C), 24.3 (2C). MS (ESI) m/z: 344.3 (M+1).

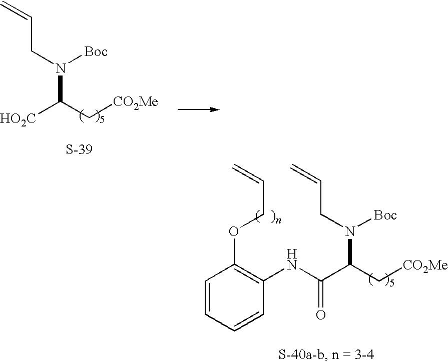

7-(S)-Allyl-tert-butoxycarbonylamino)-7-(2-pent-4-enyloxyphenylcarbamoyl)heptanoic acid methyl ester [(S)-40a]. Amide (S)-40a was obtained following the general procedure (Method 1B2) starting from acid (S)-39 (0.1 g, 0.29 mmol), DEBPT (0.17 g, 0.58 mmol), DIPEA (0.1 mL, 0.58 mmol), and aniline 22b (51 mg, 0.29 mmol) in anhydrous THF (10.0 mL). After conventional work-up and flash chromatography (9:1 hexanes/EtOAc), pure (S)-40a (95 mg, 65% yield) was isolated as a pale yellow oil: ¹H-NMR (CDCl₃, 400 MHz), mixture of atropoisomers, δ 8.66 (bs, 1H), 8.38 (d, J=7.9 Hz, 1H), 7.03 (dt, J=7.7, 1.6 Hz, 1H), 6.96 (bt, J=2.7 Hz, 1H), 6.88 (dd, J=8.0, 1.0 Hz, 1H), 6.05-6.72 (m, 1H), 6.87 (ddt, J=17.0, 10.3, 6.7 Hz, 1H), 5.81-5.03 (m, 2H), 5.10 (dd, J=17.1, 1.7 Hz, 1H), 5.04 (d, J=10.2 Hz, 1H), 4.78-4.60 (m, ½H), 4.32-4.08 (m, ½H), 4.04 (t, J=6.6 Hz, 2H), 3.91-3.68 (m, 2H), 3.68 (s, 3H), 2.35-2.25 (m, 2H), 2.33 (t, J=7.5 Hz, 2H), 2.20-2.05 (m, 1H), 2.01-2.95 (m, 2H), 1.81-1.62 (m, 4H), 1.56-1.30 (m, 12H). ¹³C-NMR (CDCl₃, 100 MHz), mixture of atropoisomers, δ 173.8, 169.0, 155.0, 146.9, 137.1, 135.2, 129.4, 127.4, 123.2, 120.5, 119.2, 116.3+115.2 (1C), 110.5, 67.4, 51.1, 36.2, 33.6 (2C), 29.6 (2C), 28.6, 27.9 (3C), 26.5, 24.2 (2C). MS (ESI) m/z: 503.3 (M+1).

7-(S)-Allyl-tert-butoxycarbonylamino)-7-(2-hex-5-enyloxyphenylcarbamoyl)heptanoic acid methyl ester [(S)-40b]. Amide (S)-40b was obtained following the general procedure (Method 1B2) starting from acid (S)-39 (0.1 g, 0.29 mmol), DEBPT (0.17 g, 0.58 mmol), DIPEA (0.1 mL, 0.58 mmol), and aniline 22c (56 mg, 0.29 mmol) in anhydrous THF (10.0 mL). After conventional work-up and flash chromatography (9:1 hexanes/EtOAc), pure (S)-40a (88 mg, 59% yield) was isolated as a pale yellow oil: ¹H-NMR (CDCl₃, 400 MHz), mixture of atropoisomers, δ 8.65 (bs, 1H), 8.38 (d, J=7.2 Hz, 1H), 7.03 (dt, J=7.8, 1.6 Hz, 1H), 6.96 (t, J=7.6 Hz, 1H), 6.87 (dd, J=8.0, 1.1 Hz, 1H), 6.05-5.76 (m, 1H), 5.85 (ddt, J=17.0, 10.2, 6.7 Hz, 1H), 5.32-4.90 (m, 2H), 5.05 (dd, J=17.1, 1.8 Hz, 1H), 5.00 (d, J=10.2 Hz, 1H), 4.73-4.61 (m, ½H), 4.33-4.05 (m, ½H), 4.03 (t, J=6.7 Hz, 2H), 3.91-3.69 (m, 2H), 3.69 (s, 3H), 2.33 (t, J=7.4 Hz, 2H), 2.16 (dd, J=14.3, 7.2 Hz, 2H), 2.16-2.00 (m, 1H), 1.95-1.80 (m, 2H), 1.78-1.56 (m, 5H), 1.48-1.30 (m, 13H). ¹³C-NMR (CDCl₃, 100 MHz), mixture of atropoisomers, δ 173.8, 169.1, 155.0, 146.9, 137.9, 123.2 (3C), 120.5, 119.2, 114.6 (2C), 110.5, 68.0, 51.1, 36.2, 33.6 (2C), 33.0 (2C), 28.6, 28.2, 26.9 (3C), 24.8, 24.4, 24.3 (2C). MS (ESI) m/z: 517.4 (M+1).

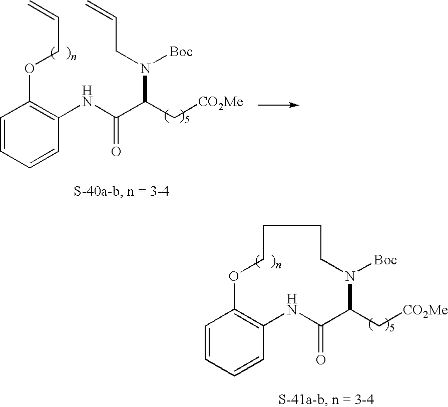

7-(S)-(5-Methoxycarbonyl-pentyl)-6-oxo-6,7,9,10,11,12,13,14-octahydro-5H-15-oxa-5,8-diazabenzocyclotridecene-8-carboxylic acid tert-butyl ester [(S)-41a]. Saturated macrocycle (S)-41a was prepared starting from the corresponding diene precursor (S)-40a (50 mg, 0.1 mmol) in a two-step sequence including the general procedure 5A followed by hydrogenation of the intermediate macrocyclic olefine. After the first step, intermediate macrocyclic olefin (40 mg, 84% yield) was obtained as a colorless oil (flash chromatography: 7:3 hexanes/EtOAc) as a mixture of isomers: ¹H-NMR (CDCl₃, 400 MHz), mixture of atropoisomers, mixture of E/Z isomers, δ 8.46 (bs, 1H), 8.31 (d, J=7.4 Hz, 1H), 7.02 (dt, J=7.6, 1.7 Hz, 1H), 6.96 (t, J=7.5 Hz, 1H), 6.83 (d, J=7.8 Hz, 1H), 6.03-5.72 (m, 2H), 4.50-4.25 (m, ½H), 4.21-4.01 (m, 2H), 3.91-3.78 (m, ½H), 3.69 (m, 3H), 3.50-3.29 (m, 1H), 2.41-2.35 (m, 2H), 2.35 (t, J=7.4 Hz, 2H), 2.19-2.09 (m, 1H), 2.02-1.96 (m, 2H), 1.72-1.62 (m, 3H), 1.49-1.32 (m, 12H). MS (ESI) m/z: 475.4 (M+1).

This macrocyclic olefin intermediate was hydrogenated according to the general procedure (Method 6A) in the presence of catalytic 3% palladium on carbon (0.1 mg/mmol) for 4 h. After flash chromatography (7:3 hexanes/EtOAc), pure (S)-41a (40 mg, 99% yield) was obtained as a colorless oil: [α]²⁰_{D} −69.3 (c 0.6, CHCl₃). ¹H-NMR (CDCl₃, 400 MHz), mixture of atropoisomers, δ 8.53 (bs, 1H), 8.34 (bs, 1H), 7.03 (t, J=7.6 Hz, 1H), 6.97 (t, J=7.5 Hz, 1H), 6.84 (d, J=7.9 Hz, 1H), 4.20-3.85 (m, 3H), 7.72-3.65 (m, 1H), 3.69 (s, 3H), 3.15-2.87 (m, 1H), 2.34 (t, J=7.4 Hz, 2H) 2.30-2.19 (m, 1H), 2.05-1.74 (m, 6H), 1.72-1.50 (m, 5H), 1.49-1.27 (m, 13H). ¹³C-NMR (CDCl₃, 100 MHz), mixture of atropoisomers, δ 173.8, 169.2, 154.9, 145.9, 127.1, 121.9, 120.7 (2C), 119.1, 109.9 (2C), 80.6, 70.0, 53.3, 51.1, 33.6 (2C), 28.8, 28.7, 27.8 (3C), 27.2, 26.3, 25.4, 24.5. MS (ESI) m/z: 477.4 (M+1).

7-(S)-(5-Methoxycarbonylpentyl)-6-oxo-6,7,10,11,12,13,14,15-octahydro-5H,9H-16-oxa-5,8-diazabenzocyclotetradecene-8-carboxylic acid tent-butyl ester [(S)-41b]. Saturated macrocycle (S)-41b was prepared starting from the corresponding diene precursor (S)-40b (45 mg, 0.09 mmol) in a two-step sequence including the general procedure 5A followed by hydrogenation of the intermediate macrocyclic olefine. After the first step, intermediate macrocyclic olefin (35 mg, 82% yield) was obtained as a colorless oil (flash chromatography: 75:25 hexanes/EtOAc) as a mixture of isomers: ¹H-NMR (CDCl₃, 400 MHz), mixture of atropoisomers, mixture of E/Z isomers, δ 8.63 (bs, 1H), 8.46 (d, J=7.5 Hz, 1H), 7.03 (bt, J=7.6 Hz, 1H), 6.99 (t, J=6.6 Hz, 1H), 6.82 (d, J=7.5 Hz, 1H), 6.00-5.75 (m, 2H), 4.58-4.21 (m, 1H), 4.05-3.95 (m, 2H), 3.92-3.82 (m, ½H), 3.80-3.71 (m, ½H), 3.69 (s, 3H), 3.57-3.39 (m, 1H), 2.35 (t, J=7.4 Hz, 2H), 2.32-2.15 (m, 2H), 2.00-1.83 (m, 2H), 1.82-1.62 (m, 4H), 1.53-1.24 (m, 15H). ¹³C-NMR (CDCl₃, 100 MHz), mixture of atropoisomers, mixture of E/Z isomers, δ 173.8, 169.3, 153.9, 147.2, 136.1, 127.4, 125.9, 125.0, 123.0 (2C), 120.5, 118.4, 110.1, 80.6, 69.1, 51.2, 33.6 (2C), 28.9, 28.7, 27.7 (3C), 26.1, 26.0, 24.5 (2C). MS (ESI) m/z: 489.4 (M+1). This macrocyclic olefin intermediate was hydrogenated according to the general procedure (Method 6A) in the presence of catalytic 3% palladium on carbon (0.1 mg/mmol) for 4 h. After flash chromatography (75:25 hexanes/EtOAc), pure (S)-41a (35 m g, 99% yield) was obtained as a colorless oil: ¹H-NMR (CDCl₃, 400 MHz), mixture of atropoisomers, δ 8.60 (bs, 1H), 8.73 (s, 1H), 7.04 (t, J=7.6 Hz, 1H), 6.96 (t, J=7.7 Hz, 1H), 5.85 (d, J=7.9 Hz, 1H), 4.23-4.10 (m, 1H), 4.09-3.89 (m, 2H), 3.82-3.51 (m, 1H), 3.69 (s, 3H), 3.20-2.85 (m, 1H), 2.51-2.34 (m, 1H), 2.34 (t, J=7.5 Hz, 2H), 2.15-1.78 (m, 4H), 1.76-1.59 (m, 5H), 1.56-1.19 (m, 17H). ¹³C-NMR (CDCl₃, 100 MHz), mixture of atropoisomers, δ 173.8, 169.5, 155.2, 146.7, 127.1, 123.0, 120.4 (2C), 118.6, 109.4 (2C), 80.6, 69.8, 62.7, 51.2, 33.7 (2C), 29.0, 28.9, 27.7 (3C), 27.6, 26.5, 24.7, 24.5 (2C). MS (ESI) m/z: 491.4 (M+1).

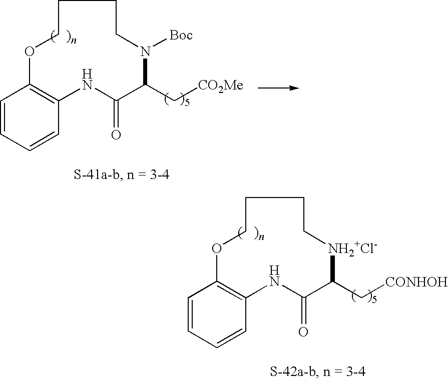

6-(6-Oxo-5,6,7,8,9,10,11,12,13,14-decahydro-15-oxa-5,8-diazabenzocyclotridecen-7-(S)yl)hexanoic acid hydroxyamide hydrochloride [(S)-42a]. Hydroxamic acid (S)-42a was prepared starting from the corresponding N-Boc protected methyl ester precursor (S)-41a (40 mg, 0.08 mmol) in a two-step sequence including the general procedure 7A followed by acidic cleavage of the N-Boc protection. After the first step, hydroxamic acid intermediate (40 mg, 99% yield) was obtained as a colorless oil: ¹H-NMR (CDCl₃, 400 MHz), mixture of atropoisomers, δ 9.50-8.50 (b, 2H), 8.54 (s, 1H), 8.31 (s, 1H), 7.03 (t, J=7.6 Hz, 1H), 6.97 (t, J=7.5 Hz, 1H), 6.84 (dt, J=7.5 Hz, 1H), 4.20-4.05 (m, 2H), 4.05-3.80 (m, 1H), 3.80-3.55 (m, 1H), 3.22-2.87 (m, 1H), 2.28-2.12 (m, 3H), 1.95-1.51 (m, 10H), 1.49-1.27 (m, 13H). ¹³C-NMR (CDCl₃, 100 MHz), mixture of atropoisomers, δ 170.8, 169.3, 155.3+155.1 (1C), 147.0, 127.3, 123.0 (2C), 118.1, 110.0 (2C), 80.7, 70.0, 63.2, 32.2, 28.6, 28.4, 28.1, 27.8 (3C), 27.1, 25.9, 25.4, 24.6, 21.4. MS (ESI) m/z: 478.4 (M+1).

This intermediate was N-deprotected according to the general procedure (Method 8A). Pure (S)-42a (95% yield) was obtained as a crystalline white solid: HPLC t_{R}=16.49 min. [α]²⁰_{D} +30.4 (c 0.3, MeOH). ¹H-NMR (CD₃OD, 400 MHz) δ 7.31 (t, J=7.3 Hz, 2H), 7.06 (d, J=8.3 Hz, 1H), 7.00 (t, J=7.5 Hz, 1H), 4.11-4.01 (m, 2H), 4.00-3.86 (m, 1H), 3.33-3.18 (m, 1H), 3.09-2.98 (m, 1H), 2.15 (t, J=7.1 Hz, 2H), 2.19-1.93 (m, 2H), 1.90-1.82 (m, 3H), 1.80-1.62 (m, 4H), 1.62-1.40 (m, 7H). ¹³C-NMR (CD₃OD, 100 MHz) δ 169.1, 166.7, 153.7, 128.4, 127.6, 124.6, 120.2, 112.5, 79.8, 68.7, 58.9, 32.2, 29.9, 28.2, 25.9, 24.9, 24.5, 24.1, 23.5, 21.7. MS (ESI) m/z: 378.3 (M+1).

6-(6-Oxo-6,7,8,9,10,11,12,13,14,15-decahydro-5H-16-oxa-5,8-diazabenzocyclotetradecen-7-(S)-yl)hexanoic acid hydroxyamide [(S)-42b]. Hydroxamic acid (S)-42b was prepared starting from the corresponding N-Boc protected methyl ester precursor (S)-41b (30 mg, 0.06 mmol) in a two-step sequence including the general procedure 7A followed by acidic cleavage of the N-Boc protection. After the first step, hydroxamic acid intermediate (30 mg, 99% yield) was obtained as a colorless oil: ¹H-NMR (CDCl₃, 400 MHz), mixture of atropoisomers, δ 9.20-8.30 (b, 2H), 8.58 (bs, 1H), 8.38 (s, 1H), 7.04 (t, J=7.6 Hz, 1H), 6.96 (t, J=7.6 Hz, 1H), 6.84 (d, J=7.9 Hz, 1H), 4.25-4.03 (m, 1H), 4.02-3.87 (m, 2H), 3.73-3.50 (m, 1H), 3.21-2.84 (m, 1H), 2.43-2.17 (m, 3H), 2.11-1.79 (m, 4H), 1.77-1.60 (m, 5H), 1.60-1.28 (m, 17H). ¹³C-NMR (CDCl₃, 100 MHz), mixture of atropoisomers, δ 172.8, 169.7, 155.3, 146.8, 126.9, 123.2, 120.4 (2C), 118.7, 109.5 (2C), 80.7, 69.8, 62.6, 29.0 (2C), 27.7 (3C), 27.5 (2C), 27.4, 26.2, 24.8, 24.5, 24.2. MS (ESI) m/z: 492.2 (M+1).

This intermediate was N-deprotected according to the general procedure (Method 8A). Pure (S)-42a (96% yield) was obtained as a crystalline white solid: ¹H-NMR (CD₃OD, 400 MHz) δ 7.31 (t, J=7.6 Hz, 2H), 7.12 (d, J=8.2 Hz, 1H), 7.00 (t, J=7.6 Hz, 1H), 4.25-4.18 (m, 1H), 4.12-4.01 (m, 2H), 3.26-3.13 (m, 1H), 3.07-2.95 (m, 1H), 2.19-2.13 (m, 2H), 2.04-1.81 (m, 4H), 1.80-1.64 (m, 5H), 1.66-1.44 (m, 9H). ¹³C-NMR (CD₃OD, 100 MHz) δ 169.0, 166.5, 152.9, 127.9, 126.5, 123.9, 119.9, 112.5, 67.5, 66.4, 59.4, 45.3, 30.2, 27.8, 26.7, 26.2, 25.5, 24.5, 24.1 22.7, 22.0. MS (ESI) m/z: 392.1 (M+1).

### Example 10

### Preparation of Macrocyclic, Heteroaromatic-Based Hydroxamic Acids Containing an Aliphatic Tether

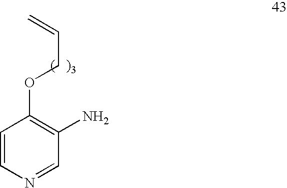

4-Pent-4-enyloxypyridin-3-ylamine (43). Amino pyridine 43 was prepared starting from 3-nitro-4-hydroxypyridine and 5-penten-1-ol in a two-step sequence including the general procedure 2C2 (flash chromatography, 1:1 hexanes/EtOAc, 60% yield) followed by the reduction of the resulting O-alkylated nitropyridine intermediate according to Method 3A2 (99% yield). 3-Nitro-4-pent-4-enyloxypyridine intermediate: ¹H-NMR (CDCl₃, 400 MHz) δ 8.99 (s, 1H), 8.60 (d, J=5.9 Hz, 1H), 7.01 (d, J=5.9 Hz, 1H), 5.82 (ddt, J=17.0, 10.3, 6.6 Hz, 1H), 5.08 (dd, J=17.1, 3.2 Hz, 1H), 5.03, (dd, J=10.2, 1.5 Hz, 1H), 4.20 (t, J=6.3 Hz, 2H), 2.28 (bdd, J=14.5, 7.0, 2H), 2.02-1.95 (m, 2H). MS (ESI) m/z: 209.1 (M+1).
43, a yellow oil: ¹H-NMR (CDCl₃, 400 MHz) δ 8.00 (s, 1H), 7.95 (d, J=5.4 Hz, 1H), 6.68 (d, J=5.4 Hz, 1H), 5.86 (ddt, J=17.0, 10.3, 6.6 Hz, 1H), 5.08 (dd, J=17.1, 1.6 Hz, 1H), 5.03 (d, J=10.3 Hz, 1H), 4.07 (t, J=6.4 Hz, 2H), 3.74 (bs, 2H), 2.26 (bdd, J=13.9, 7.0 Hz, 2H), 1.99-1.91 (m, 2H). ¹³C-NMR (CDCl₃, 100 MHz) δ 151.9, 141.0, 137.0, 136.0, 132.5, 115.2, 105.8, 66.9, 29.7, 27.7. MS (ESI) m/z: 179.1 (M+1).

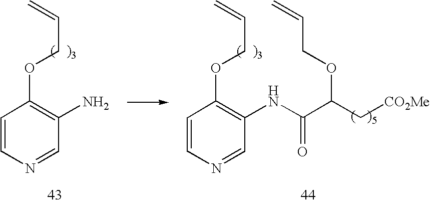

(±)-7-Allyloxy-7-(4-pent-4-enyloxypyridin-3-ylcarbamoyl)heptanoic acid methyl ester (44). Amide 44 was obtained following the general procedure (Method 1B2) starting from acid 21 (0.1 g, 0.41 mmol), DEBPT (0.25 g, 0.82 mmol), DIPEA (0.14 mL, 0.82 mmol), and aminopyridine 43 (73 mg, 0.29 mmol) in anhydrous THF (10.0 mL). After conventional work-up and flash chromatography (gradient 7:3 to 100% EtOAc in hexanes), pure 44 (75 mg, 45% yield) was isolated as a pale yellow oil: ¹H-NMR (CDCl₃, 400 MHz) δ 9.54 (s, 1H), 8.88 (s, 1H), 8.30 (s, 1H), 6.82 (d, J=5.4 Hz, 1H), 5.95 (ddt, J=17.0, 10.6, 5.5 Hz, 1H), 5.85 (ddt, J=17.0, 10.2, 6.7 Hz, 1H), 5.36 (d, J=17.2 Hz, 2H), 5.26 (d, J=10.2 Hz, 1H), 5.11-5.04 (m, 2H), 4.15-4.09 (m, 4H), 3.94 (dd, J=6.5, 4.5 Hz, 1H), 3.67 (s, 3H), 2.31 (t, J=7.4 Hz, 2H), 2.28-2.20 (m, 2H), 2.00-1.94 (m, 2H), 1.90-1.79 (m, 2H), 1.69-1.61 (m, 2H), 1.49-1.44 (m, 2H), 1.40-1.36 (m, 2H). ¹³C-NMR (CDCl₃, 100 MHz) δ 173.8, 170.4, 153.1, 145.8, 140.7, 136.6, 133.1, 117.6, 115.5, 105.8, 79.8, 71.3, 67.5, 51.1, 33.6, 32.2, 29.5, 29.3, 28.5, 27.5, 24.4, 24.1. MS (ESI) m/z: 405.3 (M+1).

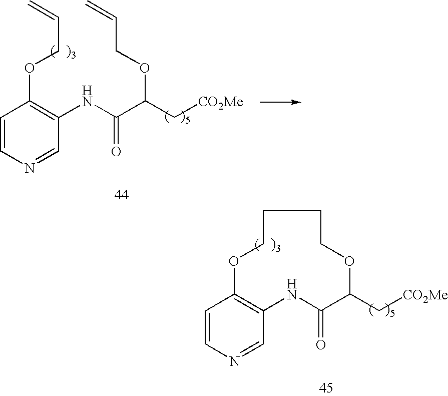

(±)-6-(14-Oxo-6,7,8,9,10,11,14,15-octahydro-13H-5,12-dioxa-2,15-diazabenzocyclotridecen-13-yl)hexanoic acid methyl ester (45). Saturated macrocycle 45 was prepared starting from the corresponding diene precursor 44 (50 mg, 0.12 mmol) in a two-step sequence including the general procedure 5A followed by hydrogenation of the intermediate macrocyclic olefine. After the first step, intermediate macrocyclic olefin (21 mg, 45% yield) was obtained as a mixture of E/Z isomers (flash chromatography: gradient MeOH in EtOAc 0 to 10%). A pale yellow oil: ¹H-NMR (CDCl₃, 400 MHz), mixture of E/Z isomers, major isomer: δ 9.57 (s, 1H), 9.08 (s, 1H), 9.29 (d, J=5.3 Hz, 1H), 6.79 (d, J=5.5 Hz, 1H), 6.07-6.01 (m, 1H), 5.76-5.64 (m, 1H), 4.46-4.40 (m, 2H), 4.03 (bt, J=9.5 Hz, 1H), 3.91 (dd, J=8.6, 3.5 Hz, 1H), 3.71-3.65 (m, 1H), 3.69 (s, 3H), 2.34 (t, J=7.4 Hz, 2H), 2.36-2.13 (m, 2H), 2.07-2.00 (m, 1H), 2.00-1.87 (m, 1H), 1.78-1.65 (m, 4H), 1.59-1.47 (m, 2H), 1.41-1.37 (m, 2H). ¹³C-NMR (CDCl₃, 100 MHz), mixture of E/Z isomers, major isomer: δ 173.7, 171.3, 152.7, 145.8, 140.4, 135.7, 129.9, 111.1, 105.5, 82.2, 71.5, 69.3, 51.1, 33.6, 33.1, 30.9, 28.5, 28.3, 21.9, 24.4. MS (ESI) m/z: 377.2 (M+1).

This macrocyclic olefin intermediate was hydrogenated according to the general procedure (Method 6A) in the presence of catalytic 3% palladium on carbon (0.1 mg/mmol) for 4 h. After flash chromatography (gradient MeOH in CH₂Cl₂ 0 to 10%), saturated macrocycle 45 (21 mg, 99% yield) was obtained as a pale yellow oil: ¹H-NMR (CDCl₃, 400 MHz) δ 9.45 (s, 1H), 9.04 (s, 1H), 8.30 (d, J=5.4 Hz, 1H), 6.86 (d, J=5.4 Hz, 1H), 4.44 (q, J=4.6 Hz, 1H), 3.99 (dt, J=9.5, 4.0 Hz, 1H), 3.77 (dd, J=8.3, 3.9 Hz, 1H), 3.69-3.57 (m, 2H), 3.68 (s, 3H), 2.34 (t, J=7.4 Hz, 2H), 1.94-1.74 (m, 6H), 1.73-1.50 (m, 8H), 1.43-1.37 (m, 2H). MS (ESI) m/z: 379.2 (M+1).

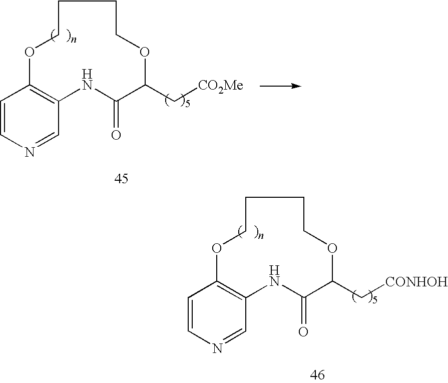

(±)-6-(14-Oxo-6,7,8,9,10,11,14,15-octahydro-13H-5,12-dioxa-2,15-diazabenzocyclotridecen-13-yl)hexanoic acid hydroxyamide (46). Hydroxamic acid 45 was prepared according to the general procedure 7A starting from the corresponding methyl ester 45 in 90% yield. After conventional work-up, the aqueous phase was concentrated in vacuo, the residue taken-up in MeOH, and the solids filtered off. After evaporation of the solvent, pure 45 was obtained as an amorphous solid: HPLC t_{R}=16.54 min. ¹H-NMR (CD₃OD, 400 MHz) δ 9.38 (s, 1H), 8.55 (d, J=6.6 Hz, 1H), 7.67 (d, J=6.5 Hz, 1H), 4.76 (q, J=5.0 Hz, 1H), 4.36 (dt, J=9.4, 4.0 Hz, 1H), 3.92 (dd, J=8.2, 4.0 Hz, 1H), 3.71-3.65 (m, 2H), 2.12 (t, J=7.3 Hz, 2H), 2.01-1.85 (m, 6H), 1.74-1.58 (m, 6H), 1.56-1.49 (m, 2H), 1.45-1.36 (m, 2H). ¹³C-NMR (CDCl₃, 100 MHz), δ 172.4, 171.0, 160.9, 138.7, 130.3, 126.9, 109.3, 80.6, 72.0, 69.7, 32.4, 31.9, 28.0, 27.9, 25.4, 25.3, 24.8, 24.3, 23.0. MS (ESI) m/z: 380.2 (M+1).

### Example 11

### Preparation of Macrocyclic Hydroxamic Acids Containing an Exocyclic Aromatic Ring

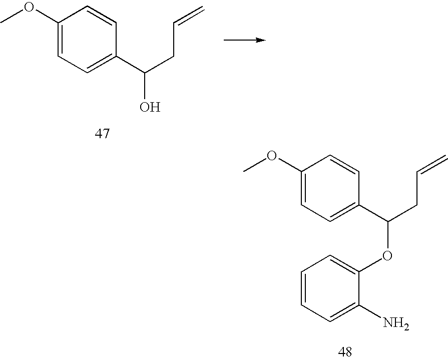

(±)-2-[1-(4-Methoxyphenyl)but-3-enyloxy]phenylamine (48). Alkoxyaniline 48 was prepared in a two-step procedure starting from 1-(4-methoxyphenyl)but-3-en-1-ol (47) including the general procedure 2C2 followed by reduction of the nitrophenoxy intermediate to aniline 48 (Method 3A1) (48% overall yield).

Alkoxynitrobenzene intermediate (69% yield), a yellow oil: ¹H-NMR (CDCl₃, 400 MHz) δ 7.77 (dd, J=8.1 Hz, 1H), 7.35-7.30 (m, 3H), 6.96-6.88 (m, 4H), 5.86 (ddt, J=17.1, 10.2, 7.1 Hz, 1H), 5.23 (dd, J=7.1, 5.8 Hz, 1H), 5.12 (m, 2H), 3.08 (s, 3H), 2.86-2.79 (m, 1H), 2.67-2.60 (m, 1H). ¹³C-NMR (CDCl₃, 100 MHz) δ 159.0, 140.0, 133.1, 133.0, 131.4, 128.4 (2C), 122.0, 125.0, 119.8, 117.9, 115.9, 113.7 (2C), 88.9, 54.9, 42.4.

Alkoxyaniline 48 (69% yield), an orange oil: ¹H-NMR (CDCl₃, 300 MHz) δ 7.34 (d, J=8.6 Hz, 2H), 6.91 (d, J=8.7 Hz, 2H), 6.78-6.74 (m, 2H), 6.66-6.56 (m, 2H), 5.91 (ddt, J=17.1, 10.2, 7.0 Hz, 1H), 5.22-5.12 (m, 3H), 4.30-3.58 (b, 2H), 3.84 (s, 3H), 2.88-2.78 (m, 1H), 2.70-2.61 (m, 1H). ¹³C-NMR (CDCl₃, 75 MHz) δ 159.9, 146.5, 137.6, 135.3, 134.4, 128.0 (2C), 122.2, 119.2, 118.5, 116.1, 114.9, 114.8 (2C), 80.8, 56.1, 43.9. MS (ESI) m/z: 270.1 (M+1).

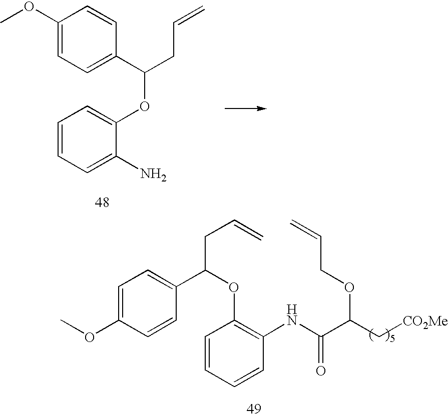

(±)-7-Allyloxy-7-[2-[1-(4-methoxyphenyl)but-3-enyloxy]-phenylcarbamoyl]-heptanoic acid methyl ester (49). Anilide 49 was obtained following the general procedure (Method 1B) starting from carboxylic acid 2i (0.15 g, 0.61 mmol), aniline 48 (0.25 g, 0.92 mmol), EDC (0.41 g, 2.14 mmol), HOBt (0.29 g, 2.14 mmol) and DIPEA (0.37 mL, 2.14 mmol) in anhydrous CH₂Cl₂ (2.0 mL). After flash chromatographic purification (gradient 95:05 to 75:25 hexanes/EtOAc) anilide 49 (0.19 g, 69% yield) was isolated as a racemic mixture of diastereomers. A pale yellow oil: ¹H-NMR (CDCl₃, 400 MHz), mixture of diastereomers (1:1), δ 9.23, 9.18 (2s, 1+1H), 8.43-8.38 (m, 2+2H), 7.27-7.74 (m, 3+3H), 6.91-6.87 (m, 2+2H), 6.77-6.73, 6.72-6.69 (2m, 1+1H), 6.06-6.96 (m, 1+1H), 5.90-5.78 (m, 1+1H), 5.41 (dd, J=17.2, 7.5 Hz, 1+1H), 5.27 (d, J=10.4 Hz, 1+1H), 5.22-5.09 (m, 3+3H), 4.26-4.19 (m, 1+1H), 4.15-4.09 (m, 1+1H), 3.94 (dd, J=11.2, 5.0 Hz, 1+1H), 3.81-3.80 (2s, 3+3H), 3.68 (s, 3+3H), 2.83-2.75 (m, 1+1H), 2.68-2.60 (m, 1+1H), 2.34 (t, J=7.4 Hz, 2+2H), 1.92-1.80 (m, 2+2H), 1.69-1.64 (m, 2+2H), 1.56-1.48 (m, 2+2H), 1.43-1.39 (m, 2+2H). ¹³C-NMR (CDCl₃, 100 MHz), mixture of diastereomers (1:1), δ 173.8 (2C), 170.5, 170.4, 158.8 (2C), 146.2 (2C), 133.5, 133.4, 133.3, 132.4, 132.0, 127.3, 127.2, 126.9 (2C), 126.7 (2C), 123.4, 123.3, 120.8, 120.7, 119.3, 119.1, 117.8, 117.7, 117.6, 117.5, 113.7 (2C), 113.5 (2C), 113.4, 112.9, 112.5, 80.2, 80.0, 79.8, 79.7, 71.4 (2C), 54.9 (2C), 51.1 (2C), 42.6, 42.2, 33.6 (2C), 32.6, 32.5, 28.6 (2C), 24.5 (2C), 24.3 (2C). MS (ESI) m/z: 496.1 (M+1).

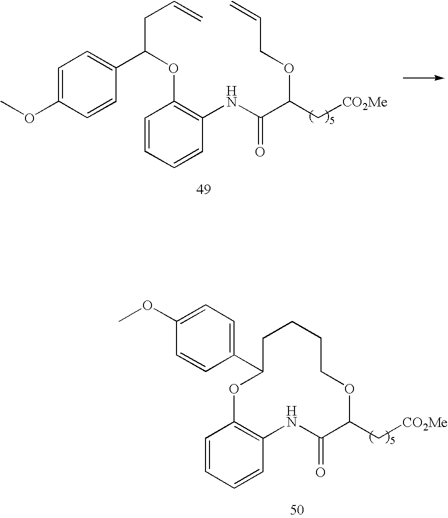

(±)-6-[13-(4-Methoxyphenyl)-6-oxo-6,7,10,11,12,13-hexahydro-5H,9H-8,14-dioxa-5-azabenzocyclododecen-7-yl]hexanoic acid methyl ester (50). Saturated macrocycle 50 was prepared starting from the corresponding diene precursor 49 (100 mg, 0.22 mmol) in a two-step sequence including the general procedure 5A followed by hydrogenation of the intermediate macrocyclic olefine in the presence of pyridine. After the first step, intermediate macrocyclic olefin (46 mg, 45% yield) was obtained as a pale yellow oil (mixture of E/Z isomers) (flash chromatography, gradient EtOAc in hexanes 10% to 30%). MS (ESI) m/z: 468.3 (M+1). This macrocyclic olefin intermediate was hydrogenated according to the general procedure (Method 6A) in the presence of catalytic 5% palladium on carbon (0.1 mg/mmol) and anhydrous pyridine (40 μL) for 4 h. After flash chromatography (gradient MeOH in CH₂Cl₂ 0 to 10%), 45 (46 mg, 99% yield) was obtained as a pale yellow oil: ¹H-NMR (CDCl₃, 400 MHz), mixture of diastereomers, δ 9.33, 9.22 (2s, 1+1H), 8.43 (d, J=6.9 Hz, 1H), 8.26 (d, J=6.9 Hz, 1H), 7.34 (d, J=8.5 Hz, 2H), 7.25 (d, J=8.5 Hz, 2H), 6.99 (t, J=8.3 Hz, 1H), 7.04-6.82 (m 4+4H), 6.49 (d, J=8.0 Hz, 1H), 5.09 (d, J=4.1 Hz, 1+1H), 4.04 (dd, J=9.5, 3.7 Hz, 1H), 3.86 (s, 3H), 3.84 (s, 3H), 3.84-3.82 (m, 1H), 3.78 (dd, J=8.6, 3.8 Hz, 1H), 3.69 (s, 3H), 3.68 (s, 3H), 3.69-3.66 (m, 1H); 3.64-3.58 (m, 1+1H), 2.36 (t, J=7.4 Hz, 2H), 2.33 (t, J=7.4 Hz, 2H), 2.25-2.10 (m, 1H), 2.08-2.01 (m, 1H), 1.98-1.86 (m, 4+4H), 1.83-1.75 (m, 1+1H), 1.73-1.64 (m, 3+3H), 1.57-1.36 (m, 5+5H). ¹³C-NMR (CDCl₃, 100 MHz), mixture of diastereomers, δ 173.8 (2C), 171.5, 170.7, 158.8, 158.6, 148.2, 147.3, 132.7, 132.5, 130.3, 130.0, 127.4 (2C), 126.6 (2C), 123.9, 123.8, 123.6, 123.0, 120.4, 120.2, 120.1, 119.5, 113.4 (2C), 113.3 (2C), 85.2 (2C), 81.7, 80.3, 68.3, 65.5, 54.9 (2C), 51.1 (2C), 34.1, 33.6 (2C), 33.4, 32.5, 30.2, 28.5, 28.5, 28.4, 27.1, 25.1, 25.0, 24.5, 24.4, 19.2, 16.6. MS (ESI) m/z: 470.1 (M+1).

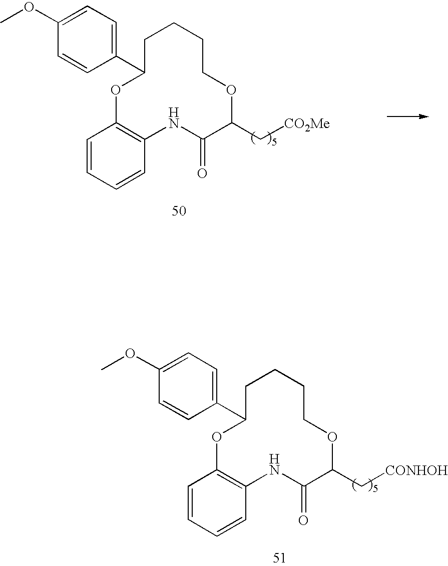

(±)-6-[13-(4-Methoxyphenyl)-6-oxo-6,7,10,11,12,13-hexahydro-5H,9H-8,14-dioxa-5-azabenzocyclododecen-7-yl]-hexanoic acid hydroxyamide (51). Hydroxamic acid 51 was prepared according to the general procedure 7A starting from the corresponding methyl ester 50 in 93% yield. A colorless oil: HPLC t_{R}=6.34 min (minor), 6.55 (major). ¹H-NMR (δ₆-DMSO, 400 MHz), mixture of diastereomers, δ 10.37 (bs, 1+1H), 9.28 (d, J=5.5 Hz, 1+1H), 8.69 (bs, 1+1H), 8.22 (d, J=7.4 Hz, 1H), 7.76 (d, J=7.4 Hz, 1+1H), 7.35-7.29 (m, 2+2H), 7.06 (t, J=7.5 Hz, 1H), 6.98-6.87 (m, 3+4H), 6.75 (d, J=7.7 Hz, 1H), 6.51 (d, J=7.0 Hz, 1H), 5.07 (bs, 1+1H), 4.17-4.15 (m, 1H), 4.02-3.99 (dd, J=8.5, 4.6 Hz, 1H), 3.77 (s, 3H), 3.76 (s, 3H), 3.76-7.71 (m, 1H), 3.68-3.53 (m, 2+1H), 1.98-1.93 (m, 2+2H), 1.83-1.59 (m, 4+4H), 1.54-1.47 (m, 3+3H), 1.43-1.36 (m, 2+2H), 1.34-1.27 (m, 5+5H). ¹³C-NMR (CDCl₃, 100 MHz), mixture of diastereomers, δ 171.4, 171.2, 169.1 (2C), 158.7, 158.6, 149.9, 148.3, 133.7, 133.1, 130.5, 129.6, 127.4 (2C), 127.3 (2C), 125.2, 124.3, 123.7, 123.1, 122.3, 121.1, 119.8, 118.9, 113.8 (2C), 113.7 (2C), 84.6, 84.3, 81.2, 80.1, 68.8, 66.5, 55.1 (2C), 35.1, 33.2, 33.1, 32.3 (2C), 30.6, 28.5, 28.4, 27.2 (2C), 25.1 (2C), 24.8 (2C), 20.0, 17.3. HRMS (ES+) C₂₆H₃₄N₂O₆ calcd for [MH]⁺ 471.24896. found 451.24826.

### Generic Examples 12

### Preparation of Macrocyclic Hydroxamic Acids Containing an Extra Amino Group on the Anilide Ring

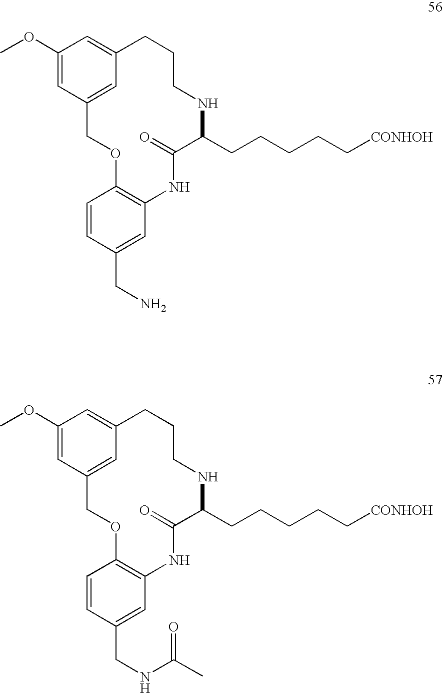

Macrocycles 56, 57 and analogues thereof can be prepared starting from benzyloxyaniline 55 following the general multistep sequence described in Chart 1. Benzyloxyaniline 55 can be prepared from commercially available 3-amino-4-hydroxy-benzoic acid (52) in a six-step sequence (Scheme 11) including N-protection (e.g. Boc₂O, CH₂Cl₂, Et₃N), O-protection (e.g. TBSCl, imidazole, CH₂Cl₂), reduction (e.g. BH₃⁻THF), azide formation under modified Mitsunobu conditions (DPPA, PPh₃, DIAD; Hughes, D. L. Org. Prep. Proceed. Int. 1996, 28, 127; Mitsunobu, O. Synthesis 1981, 1) or under Merck conditions (DPPA, DBU, THF; Thompson, A. S.; Humphrey, G. R.; DeMarco, A. M.; Mathre, D. J. Grabowski, E. J. J. J. Org. Chem. 1993, 58, 5886-5888), O-deprotection (e.g. TBAF, THF), O-alkylation (Method 2C2) and following manipulation (PDC, then Wittig, see under Example 9 conversion of 27 to 29, Scheme 7), N-Boc deprotection (Method 8A). Azide reduction (H₂, Pd—C or PPh₃, THF, H₂O, Golobolov, Y. G.; Kasukhin, L. F. Tetrahedron 1992, 48, 1353-1406), N-protection (e.g. Boc₂O, CH₂Cl₂) or N-acetylation (Ac₂O, py, DMAP), can be performed after the coupling step.

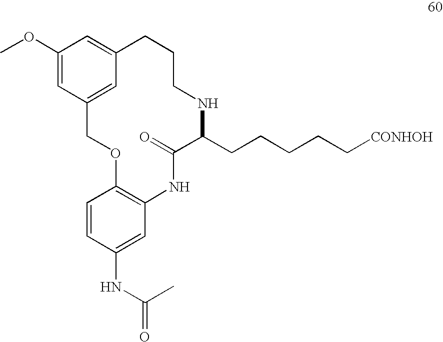

Macrocycle 60 and analogues thereof can be prepared from benzyloxyaniline 59 following the general multistep sequence described in Chart 1. Aniline 59 can be prepared in a five-step sequence (Scheme 12) including Curtius rearrangement of N,O-diprotected benzoic acid 53 (Smith, P. A. S. Org. React. 1946, 337-349; Capson, T. L.; Poulter, C. D. Tetrahedron Lett. 1984, 25, 3515-3518; see also: Tichenor, M. S.; Trzupek, J. D.; Kastrinsky, D. B.; Shiga, F.; Hwang, I.; Boger, D. L. J. Am. Chem. Soc. 2006, 128, 15683-15696) followed by N-acetylation (Ac₂O, Py, DMAP), O-deprotection (e.g. TBAF, THF), O-alkylation (Method 2C2) and following manipulation (PDC, then Wittig, see under Example 9 conversion of 27 to 29, Scheme 7), N-deprotection (Method 8A).

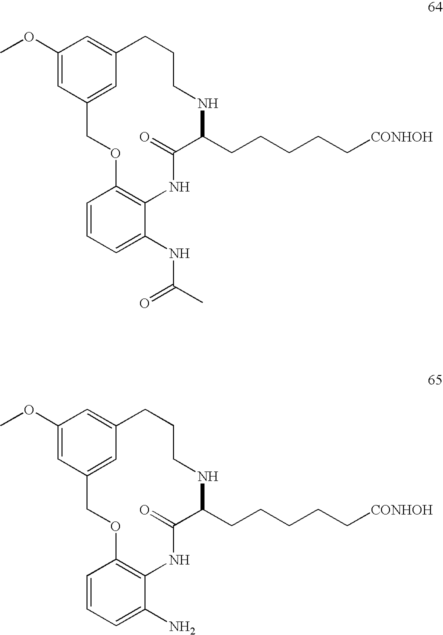

Macrocycles 64 and 65 and analogues thereof can be prepared starting from commercially available 2-amino-3-hydroxybenzoic acid (6i) (Scheme 13), following the procedures above described for compound 60.

### Generic Examples 13

### Preparation of Pyridine-, Pyrazolo-, and Pyrrole-Based Macrocyclic Hydroxamic Acids

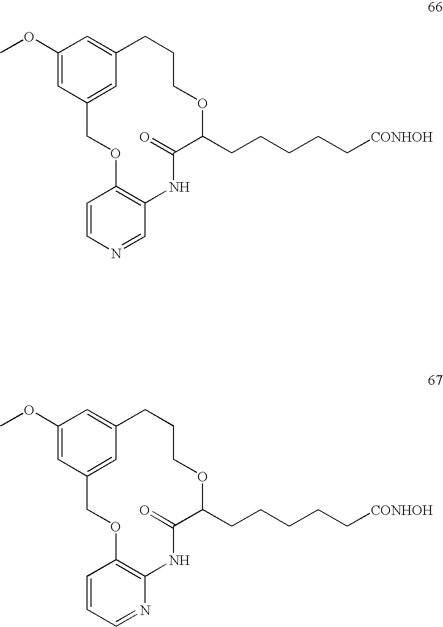

Macrocycles 66 and 67 and analogues thereof can be prepared starting from commercially available 3-amino-4-hydroxypyridine and 2-amino-3-hydroxypyridine respectively, following the general multistep sequence described in Chart 1. For specifications, see also compound 46, Scheme 9, Example 10.

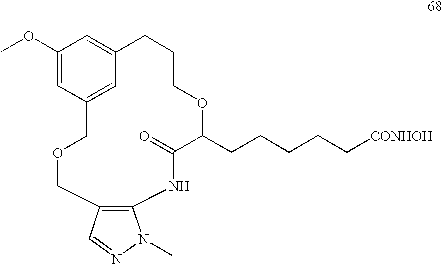

Macrocycle 68 and analogues thereof can be prepared starting from 4-(hydroxymethyl)-1-methyl-1H-5-nitropyrazole (Hay, M.; Anderson, R. F.; Ferry, D. M.; Wilson, W. R.; Denny, W. A. J. Med. Chem. 2003, 46, 5533; Cheng, C.-C. J. Heterocycl. Chem. 1972, 15, 1035) following the general multistep sequence described in Chart 1.

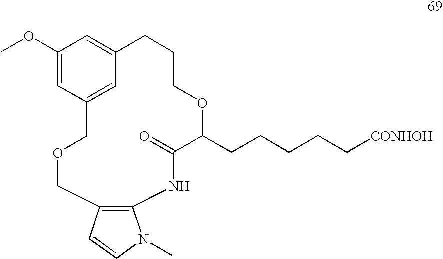

Macrocycle 69 and analogues thereof can be prepared starting from 3-hydroxymethyl-1-methyl-1H-2-nitropyrrol (Hay, M.; Anderson, R. F.; Ferry, D. M.; Wilson, W. R.; Denny, W. A. J. Med. Chem. 2003, 46, 5533; Tercel, M.; Lee, A. E.; Hogg, A.; Anderson, R. F.; Lee, H. H.; Siim, B. G.; Denny, W. A.; Wilson, W. R. J. Med. Chem. 2001 44, 3511) following the general multistep sequence described in Chart 1.

### Generic Examples 14

### Preparation of Indole-Based Macrocyclic Hydroxamic Acids

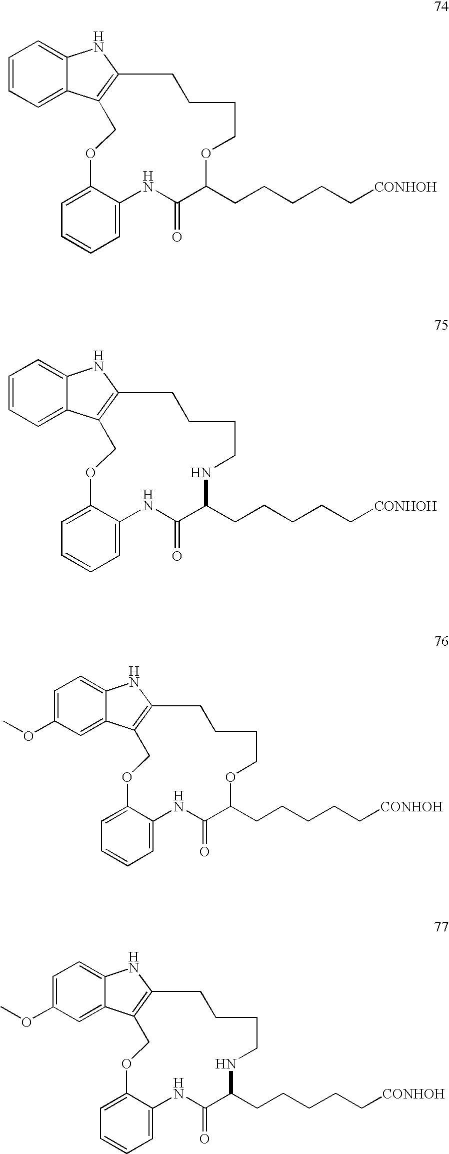

Macrocycles 74-77 and analogues thereof can be prepared starting from suitable (indol-3-ylmethoxy)anilines 73 (Scheme 14) according to the general multistep sequence described in Chart 1. Anilines 73 can be prepared in turn by formylation with Cl₂CHOMe under TiCl₄ promotion of suitable substituted 2-allylindols (Bennasar, M.-L.; Zulaica, E.; Tummers, S. Tetrahedron Lett. 2004, 45, 6283-6285. For C2-allylation of substituted indols, see: Hanessian, S.; Giroux, S.; Larsson, A. Org. Lett. 2006, 8, 5481-5485; for the synthesis of substituted indols, see: Mahboobi, S.; Uecker, A.; Sellmer, A.; Cénac, C.; Höcher, H.; Pongratz, H.; Eichhorn, E.; Hufsky, H.; Trümpler, A.; Sicker, M.; Heidel, F.; Fisher, T.; Stocking, C.; Elz, S.; Böhmer, F.-D.; Dove, S. J. Med. Chem. 2006, 49, 3101-3115; Prieto, M.; Zurita, E.; Rosa, E.; Muñoz, L.; Lloyd-Williams, P.; Giralt, E. J. Org. Chem. 2004, 69, 6812-6820), followed by reduction of the resulting aldehyde to alcohol (NaBH4), O-alkylation with o-nitrophenol (Method 2C2), and reduction to aniline derivative (Method 3A2).

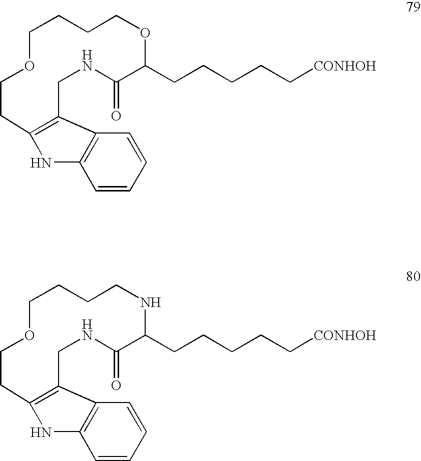

Macrocycles 79, 80 can be prepared starting from the suitable 2-(2-allyloxyethyl)-3-methylamino indols 78 (Scheme 15) according to the general multistep sequence described in Chart 1. Indols 78 can be prepared from the suitable 2-allyl-3-hydroxymethyl indols 72 in a 4-step sequence including conversion to azide under Merck conditions (DPPA, DBU, THF; Thompson, A. S.; Humphrey, G. R.; DeMarco, A. M.; Mathre, D. J. Grabowski, E. J. J. J. Org. Chem. 1993, 58, 5886-5888), double bond oxidative cleavage followed by reduction (OsO₄, NaIO₄, then NaBH₄; or O₃, then NaBH₄ Hudlicky, M. Oxidation in Organic Chemistry, American Chemical Society, Washington, D.C., 1990), O-allylation (NaH, allyl iodide), and azide reduction under Staudinger conditions (PPh₃, H₂O, THF, Golobolov, Y. G.; Kasukhin, L. F. Tetrahedron 1992, 48, 1353-1406).

### Generic Examples 15

### Preparation of Macrocyclic Hydroxamic Acids Containing an Extra Amino Group on the Suberoyl Chain

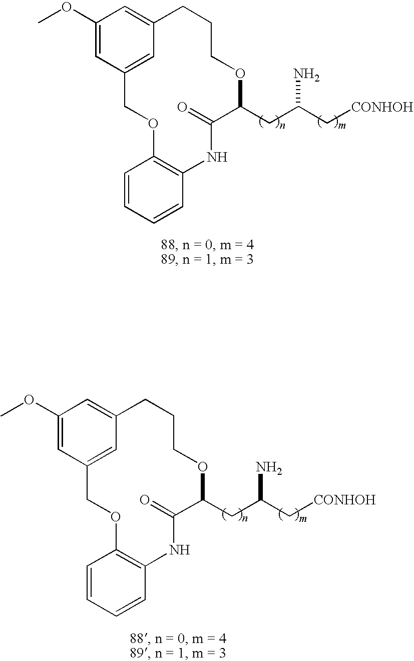

### Complete Description

Macrocycles 88, 88′, and 89, 89′, their enantiomers, and analogues thereof, can be prepared starting from carboxylic acids 86, 86′, and 87, 87′, following the general multistep sequence described in Scheme 16, including for example, coupling with benzyloxy aniline 29 (Method 1B1 or 1B2), ring closing metathesis (Method 5A), hydroxamic acid formation (Method 7A), azide and double bond concomitant reduction (H₂, Pd—C).
Carboxylic acids 86, 87 can be prepared from enantiopure 2,3-O-isopropylidene glyceraldehyde 81 (commercial) and 3,4-O-isopropylidene-3,4-dihydroxybutanal (from oxidation of commercial 4-(2-hydroxymethyl)-2,2-dimethyl-1,3-dioxolane, e.g. PDC, CH₂Cl₂) respectively, in a sequence including stereoselective C-allylation according to the Brown procedure [(+)- or (−)-Ipc₂Ballyl, H₂O₂, NaOH, (a) Srebnik, M.; Rachamandran, P. V. Aldrichimica Acta, 1987, 20, 9-24. (b) Roush, W. R. In Comprehensive Organic Synthesis, Trost, B. M.; Fleming, I., Eds, Pergamon Press: New York, 1991, Vol. 2, pp. 1-53; synthesis of 84: Nicolaou, K. C.; Pihko, P. M.; Bernal, F.; Frederick, M. 0.; Qian, W.; Uesaka, N.; Diedrichs, N.; Hinrichs, J.; Koftis, T. V.; Loizidou, E.; Petrovic, G.; Rodriguez, M.; Sarlah, D.; Zou, N. J. Am. Chem. Soc. 2006, 128, 2244], C2 or C3 homologation via cross metathesis with the suitable olefin 83a or 83b and following hydrogenation of the double bond (Methods 4A, 6A), azide formation through inversion of configuration at C3 or C4 (DPPA, DIAD, PPh₃, Hughes, D. L. Org. Prep. Proceed. Int. 1996, 28, 127; Mitsunobu, O. Synthesis 1981, 1), removal of the acetonide protection (AcOH), selective protection of the primary alcohol (TBDPSCl, imidazole), O-allylation of the secondary alcohol (allyl trichloroacetimidate, TfOH), desilylation (TBAF) and oxidation of the primary alcohol (Swern, then Jones oxidation) (for a specific example of this sequence, see Scheme 6, compound 20). Isomers 88′ and 89′ can be prepared from 84, 85 after Mitsunobu inversion of configuration at C3 or C4 to 84′, 85′.

‘Optional’ Description (Condensed, Referring to CHART 1)

Macrocycles 88, 88′, and 89, 89′, their enantiomers, and analogues thereof, can be prepared starting from carboxylic acids 86, 87, (Scheme 16), following the general multistep sequence described in Chart 1.

Carboxylic acids 86, 87 can be prepared from enantiopure 2,3-O-isopropylidene glyceraldehyde 81 (commercial) and 3,4-O-isopropylidene-3,4-dihydroxybutanal 82 (from oxidation of commercial 4-(2-hydroxymethyl)-2,2-dimethyl-1,3-dioxolane, e.g. PDC, CH₂Cl₂) respectively, in a sequence including stereoselective C-allylation according to the Brown procedure [(+)- or (−)-Ipc₂Ballyl, H₂O₂, NaOH, (a) Srebnik, M.; Rachamandran, P. V. Aldrichimica Acta, 1987, 20, 9-24. (b) Roush, W. R. In Comprehensive Organic Synthesis, Trost, B. M.; Fleming, I., Eds, Pergamon Press: New York, 1991, Vol. 2, pp. 1-53; synthesis of 84: Nicolaou, K. C.; Pihko, P. M.; Bernal, F.; Frederick, M. 0.; Qian, W.; Uesaka, N.; Diedrichs, N.; Hinrichs, J.; Koftis, T. V.; Loizidou, E.; Petrovic, G.; Rodriguez, M.; Sarlah, D.; Zou, N. J. Am. Chem. Soc. 2006, 128, 2244], C2 or C3 homologation via cross metathesis with the suitable olefin 83a or 83b and following hydrogenation of the double bond (Methods 4A, 6A), azide formation through inversion of configuration at C3 or C4 (DPPA, DIAD, PPh₃, Hughes, D. L. Org. Prep. Proceed. Int. 1996, 28, 127; Mitsunobu, O. Synthesis 1981, 1), removal of the acetonide protection (AcOH), selective protection of the primary alcohol (TBDPSCl, imidazole), O-allylation of the secondary alcohol (allyl trichloroacetimidate, TfOH), desilylation (TBAF) and oxidation of the primary alcohol (Swern, then Jones oxidation) (for a specific example of this sequence, see Scheme 6, compound 20). Isomers 88′ and 89′ can be prepared from 84, 85 after Mitsunobu inversion of configuration at C3 or C4 to 84′, 85′.

### Example 16

### Cytotoxicity Studies

To test the effects of the compounds on cell growth, NB4 human promyelocitic leukaemia, NCI-H460 non-small cell carcinoma cells and HCT-116 human colon carcinoma cells were used. NB4 and NCI-H460 tumor cells were grown RPMI 1640 containing 10% fetal bovine serum (GIBCO), whereas HCT-116 tumor cells were grown in McCoy's 5A containing 10% fetal bovine serum (GIBCO).

Tumor cells were seeded in 96-well tissue culture plates at approximately 10% confluence and were allowed to attach and recover for at least 24 h. Varying concentrations of the drugs were then added to each well to calculate their IC50 value (the concentration which inhibits the 50% of cell survival). The plates were incubated for 24 h at 37° C. At the end of the treatment, for NB4 tumor cells in suspension, the procedure was performed as follows: medium culture was removed by centrifugation of the plates at 1600×g for 10 min and the surnatant was removed. 250 μl PBS were added, then the plates were centrifuged at 1600×g for 10 min, the surnatant was removed. 200 μl/well of medium culture RPMI 1640 containing 10% FCS were added and the plates were incubated at 37° C. for other 48 h. The plates were centrifuged again at 1600×g for 10 min, the medium culture was removed and 200 μl PBS and 50 μl of cold 80% TCA were added. The plates were incubated on ice for at least 1 h. TCA was removed, the plates were washed 3 times for immersion in distilled-water and dried before on paper, then in a thermostater at 40° C. Subsequently 200 μl of 0.4% sulphorodamine B in 1% acetic acid were added. The plates were incubated at room temperature for other 30 min. Sulphorodamine B was removed, the plates were washed for immersion in 1% acetic acid for 3 times, then they were washed and dried on paper. 200 μl Tris 10 mM were added, the plates were kept under stirring for 20 min. The survival cell was determined as optical density by a Multiskan spectrofluorimeter at 540 nm. For the tumor cells in adhesion (NCI-H460 and HCT-116), the procedure was as above mentioned, except that the at the end of the treatment, the plates were washed by remotion of the surnatant and addition of PBS 3 times and not by centrifugation. Also the last day of the assay, the surnatant was removed without centrifugation.

The amount of cells killed was calculated as the percentage decrease in sulphorodamine B binding compared with control cultures. The IC50 values (the concentration which inhibits the 50% of cell survival) were calculated with the “ALLFIT” program.

In the table 1 the cytotoxicity evaluated on NB4 tumor cells showed that the compounds were slightly more active on NB4 promyelocytic leukemia cells than NCI-H460 and HCT116 cells (non-small cell lung and colon carcinoma, respectively). Upon 24 h of treatment, the compounds revealed an antiproliferative effect with IC50 values ranging from 0.05 μM to 20 μM. In particular, many compounds had a mean IC50 value<1 μM on the three tumor cell lines such as 9a, 9b, 9d, (S)-9d, (R)-9d, 9e, 9f, 9g, 9h, 9j, 9k, 9l, 9m, 13d, 26b, 26c, 32, 34 (ST3265, ST3267, ST3269, ST3339, ST3338, ST3429, ST3430, ST3431, ST3432, ST3434, ST3435, ST3436, ST3437, ST3270, ST3533, ST3534, ST3615, ST3616).

TABLE 1
Cytotoxicity of different compounds on NB4, NCI-H460 and HCT-116
tumor cells

| | | NB4 | NCI-H460 | HCT-116 |
|---|---|---|---|---|
| Compound | | IC50 ± SD, μM | | |
| 5a | ST3239 | 3.7 ± 0.6 | 13.7 ± 2.6 | 8.6 ± 0.2 |
| 5b | ST3234 | 3.5 ± 0.4 | 5.7 ± 0.5 | 5.0 ± 0.1 |
| (S)-5b | ST3336 | 3.5 ± 0.3 | 7.8 ± 0.5 | 6.8 ± 0.3 |
| (R)-5b | ST3337 | 4.0 ± 0.6 | 9.6 ± 0.5 | 7.8 ± 0.5 |
| 5c | ST3236 | 4.5 ± 0.4 | 8.6 ± 0.3 | 4.3 ± 0.2 |
| 5d | ST3235 | 9.6 ± 0.8 | 14.7 ± 1.6 | 12.4 ± 0.2 |
| 5e | ST3233 | 4.1 ± 0.3 | 10.4 ± 1.2 | 8.5 ± 0.3 |
| 5f | ST3238 | >5 | 5.5 ± 0.6 | 2.4 ± 0.06 |
| 5g | ST3237 | 1.0 ± 0.1 | 2.0 ± 0.3 | 1.3 ± 0.06 |
| 9a | ST3265 | 0.5 ± 0.03 | 1.0 ± 0.09 | 0.7 ± 0.02 |
| 9b | ST3267 | 0.5 ± 0.03 | 1.2 ± 0.1 | 0.9 ± 0.04 |
| 9c | ST3271 | 0.4 ± 0.02 | 1.8 ± 0.1 | 1.0 ± 0.1 |
| 9d | ST3269 | 0.1 ± 0.01 | 0.5 ± 0.02 | 0.2 ± 0.006 |
| (S)-9d | ST3339 | 0.1 ± 0.002 | 0.4 ± 0.03 | 0.2 ± 0.007 |
| (R)-9d | ST3338 | 0.07 ± 0.01 | 0.5 ± 0.03 | 0.4 ± 0.01 |
| 9e | ST3429 | 0.2 ± 0.002 | 0.5 ± 0.06 | 0.4 ± 0.03 |
| 9f | ST3430 | 0.06 ± 0.004 | 0.5 ± 0.04 | 0.3 ± 0.05 |
| 9g | ST3431 | 0.1 ± 0.01 | 0.6 ± 0.01 | 0.3 ± 0.009 |
| 9h | ST3432 | 0.2 ± 0.007 | 0.6 ± 0.03 | 0.6 ± 0.1 |
| 9i | ST3433 | 4.0 ± 0.7 | 7.4 ± 0.5 | 7.5 ± 0.4 |
| 9j | ST3434 | 0.05 ± 0.005 | 0.4 ± 0.07 | 0.3 ± 0.008 |
| 9k | ST3435 | 0.2 ± 0.009 | 0.7 ± 0.04 | 0.7 ± 0.08 |
| 9l | ST3436 | 0.2 ± 0.02 | 1.0 ± 0.09 | 0.5 ± 0.06 |
| 9m | ST3437 | 0.1 ± 0.04 | 0.3 ± 0.04 | 0.3 ± 0.01 |
| 13a | ST3266 | 0.9 ± 0.1 | 3.6 ± 0.8 | 1.6 ± 0.05 |
| 13b | ST3268 | 0.8 ± 0.03 | 1.9 ± 0.1 | 1.1 ± 0.009 |
| 13c | ST3272 | 0.6 ± 0.06 | 1.5 ± 0.3 | 1.0 ± 0.05 |
| 13d | ST3270 | 0.3 ± 0.03 | 0.7 ± 0.03 | 0.5 ± 0.1 |
| 26a | ST3532 | 0.6 ± 0.1 | 2.0 ± 0.1 | 0.7 ± 0.1 |
| 26b | ST3533 | 0.3 ± 0.03 | 1.0 ± 0.05 | 0.6 ± 0.03 |
| 26c | ST3534 | 0.4 ± 0.02 | 0.6 ± 0.02 | 0.3 ± 0.03 |
| 32 | ST3615 | nd | 0.9 ± 0.08 | 0.4 ± 0.03 |
| 34 | ST3616 | nd | 0.9 ± 0.06 | 0.7 ± 0.01 |
| (S)-42a | ST5511CL1 | nd | 1.1 ± 0.07 | 0.8 ± 0.07 |
| 46 | ST5512AA1 | nd | 20 | 13 ± 1.7 |

nd = not determined.

1. Compounds of Formula 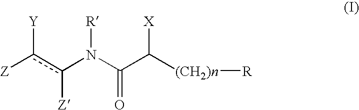
and their geometrical isomers, in an optically active form as enantiomers, diastereomers, as well as in the form of racemate, as well as pharmaceutically acceptable salts thereof, wherein:
the dotted line indicates an optional double bond;
R is CONHOH, CONHCH₂SH, CONHCH₂SCOCH₃, SH, SCOCH₃, SCH₃, N(OH)COH, COCONHCH₃, or CF₃;
n=1-7 and the alkylene chain is unsubstituted or substituted with one or more NH₂ groups, OH, (C₁₋₃)alkyl, SH, or (C₁₋₃)alkoxy;
z and z′ are linked to form a phenyl group or a five- or six-membered heteroaromatic ring containing one to four nitrogen atoms, the phenyl group or the five- or six-membered heteroaromatic ring being unsubstituted or substituted with up to 4 substituents R″ or optionally condensed with an aryl or heteroaryl group;
X is selected from the group consisting of OH, unsubstituted or substituted (C₁₋₇)-alkoxy group, O—CH₂-Aryl, where aryl is unsubstituted or substituted with one or two substituents, which are the same or different and are selected from the group consisting of H, NH₂, NH—(C₁₋₃)Alkyl, CN, NO₂, (C₁₋₃)Alkyl unsubstituted or substituted with halogen, O—(C₁₋₃)Alkyl, Halogen, aryl, O-Aryl;
Y is selected from the group comprising H, OH, O—(C₁₋₃)Alkyl, NH₂, NH—(C₁₋₃)Alkyl, Halogen;
Or X and Y form a cycle wherein X and Y are linked by a bridge of Formula A selected from the group consisting of:
X—(C₁₋₄)Alkylene(R1)-W—(C₁₋₄)Alkylene-Y
X—(C₂₋₄)Alkenylene(R1)-W—(C₁₋₄)Alkylene-Y
wherein X and Y are the same or different and are selected from the group consisting of —O—, —NH— unprotonated or protonated, —S—, —CH₂—, (C₁₋₃)-Alkylene-O—;
W is either absent or it represents an arylene group selected from the group comprising: 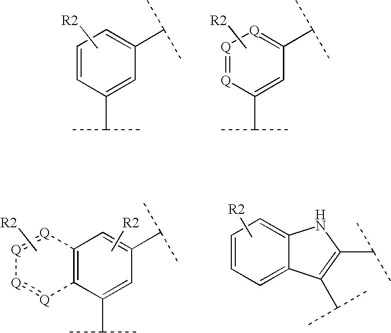
R′ represents H, (C₁₋₅)Alkyl, CH₂— Aryl unsubstituted or substituted with H, O—(C₁₋₃)Alkyl, OH and nitro;
R″ represents H, NH₂, NH—(C₁₋₃)Alkyl, NHCO(C₁₋₃)Alkyl, O—(C₁₋₃)Alkyl, (C₁₋₃)Alkylene-NH₂, (C₁₋₃)Alkylene-NHCO(C₁₋₃)Alkyl, (C₁₋₃)Alkyl, NH-acyl, (C₁₋₃)Alkylene-NH-acyl, OH;
R1 represents H, halogen, NO₂, (C₁₋₃)Alkyl-NH₂, OH, NH₂ unsubstituted or substituted with a (C₁₋₃)acyl group, phenyl group unsubstituted or substituted with a —O—(C₁₋₃)Alkyl;
R2 represents H, (C₁₋₅)Alkyl, —O—(C₁₋₃)Alkyl, halogen, NO₂, NH₂ unsubstituted or substituted with a (C₁₋₃)acyl group or a (C₁₋₃)Alkyl, OH. CN, COOR3 where R3 is selected from the group consisting of H, (C₁₋₃)Alkyl; and
Q represents CH, N or, for saturated derivatives, CH₂, NH.

2. Compounds according to claim 1, having the Formula II: 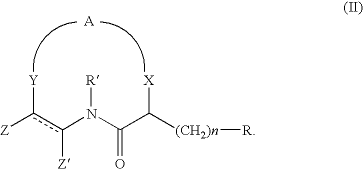
wherein X and Y form a cycle and wherein z, z′, Y, A, X, n, R′ and R are as defined in claim 1.

3. Compounds according to claim 1, characterized in that the bridge of Formula A is selected from the group consisting of —(CH₂)₃—, —(CH₂)₄—, —(CH₂)₅—, and

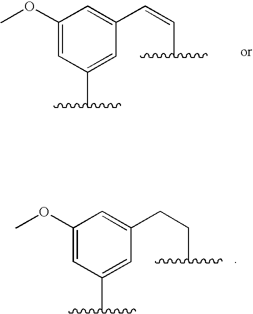

4. Compounds according to claim 1, characterized in that R is CONHOH.

5. Compounds according to claim 1, characterized in that n is from 4 to 6.

6. Compounds according to claim 1, characterized in that z and z′ are linked to form a phenyl group or a five- or six-membered heteroaromatic ring selected from the group comprising pyridine, pyrazole and pyrrole.

7. Compounds according to claim 1, characterized in that R″ is selected from the group consisting of H, —CH₃, —OCH₃, —NHCOCH₃, —NH₂, —CH₂NH₂, —CH₂NHCOCH₃.

8. Compounds according to claim 1, characterized in that it is selected from the group consisting of:

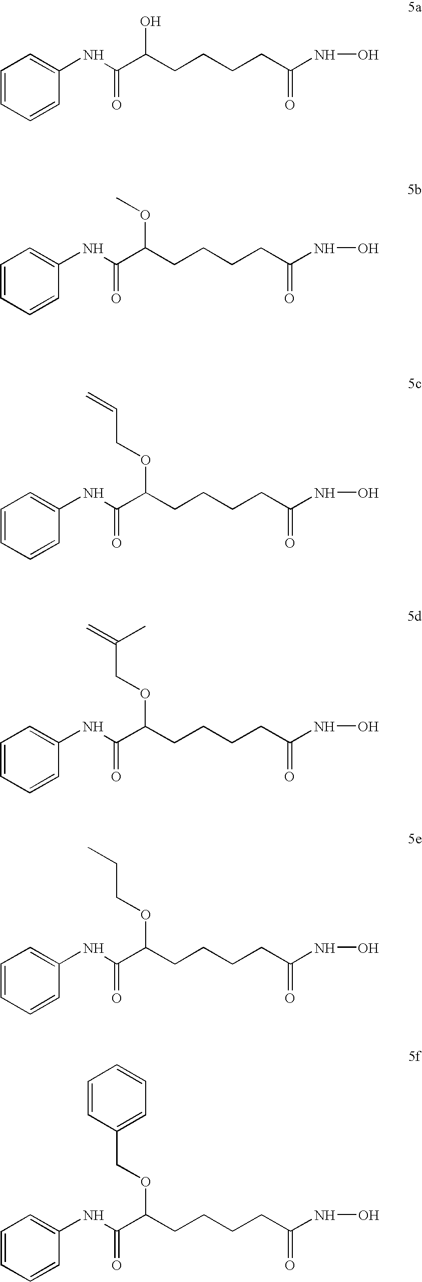

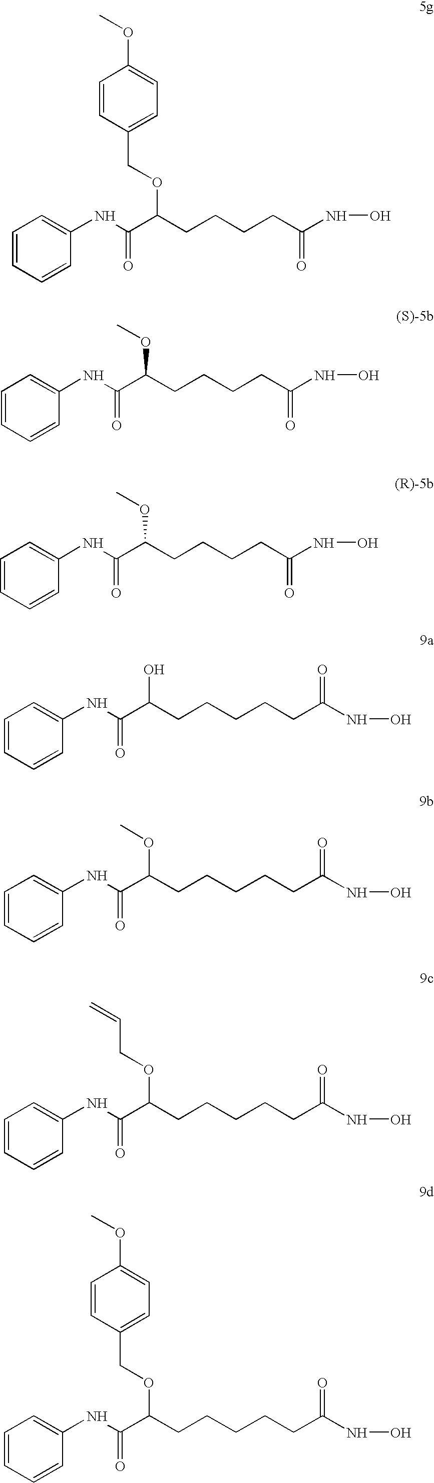

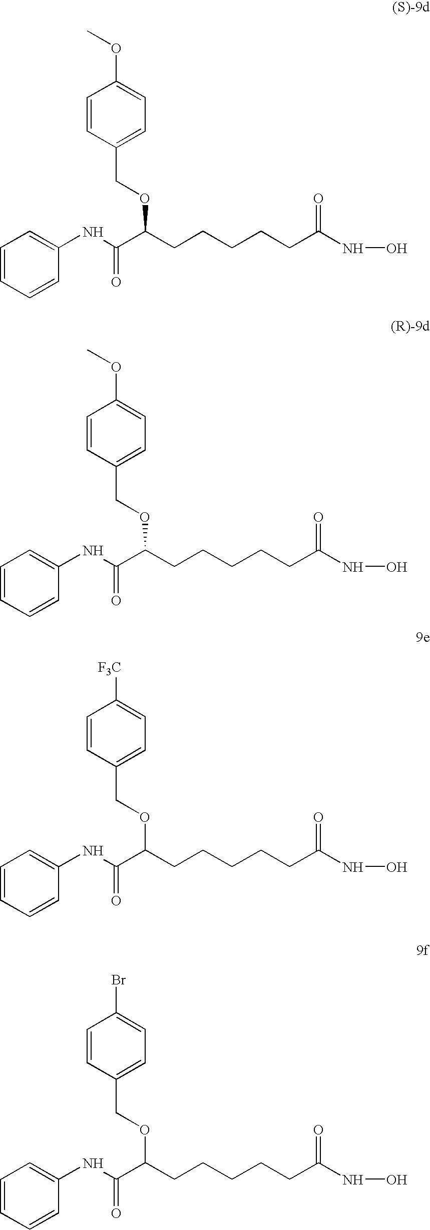

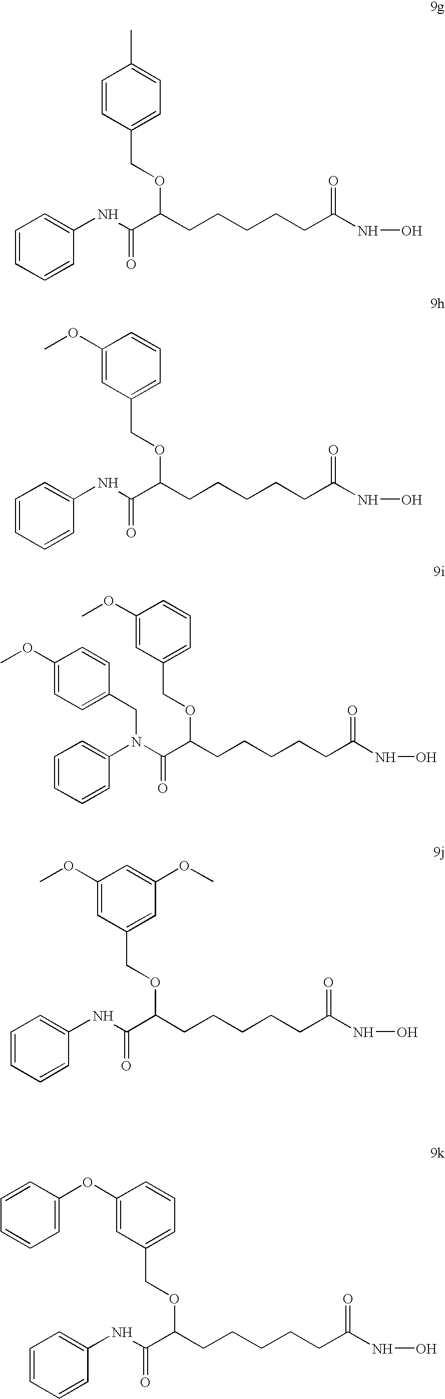

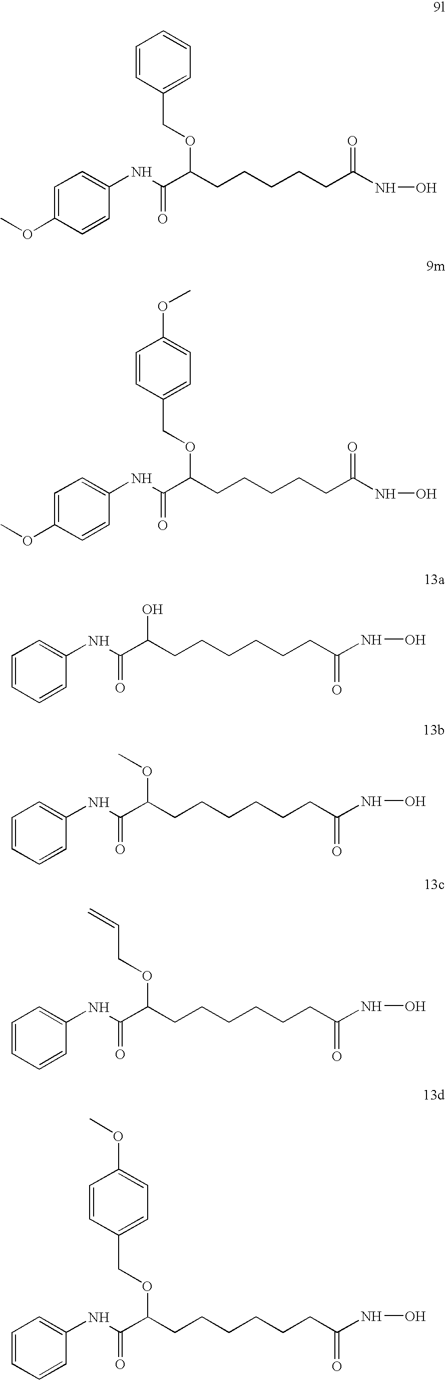

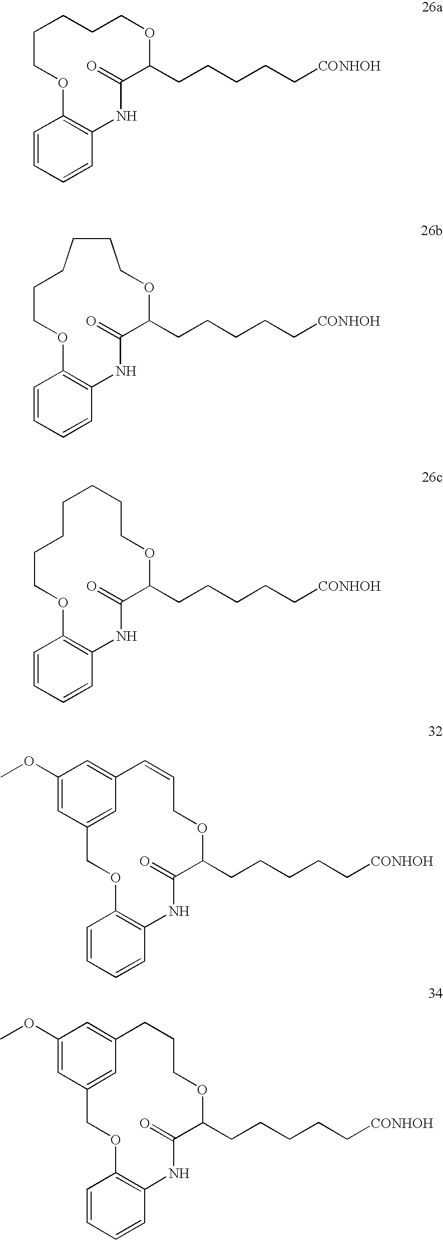

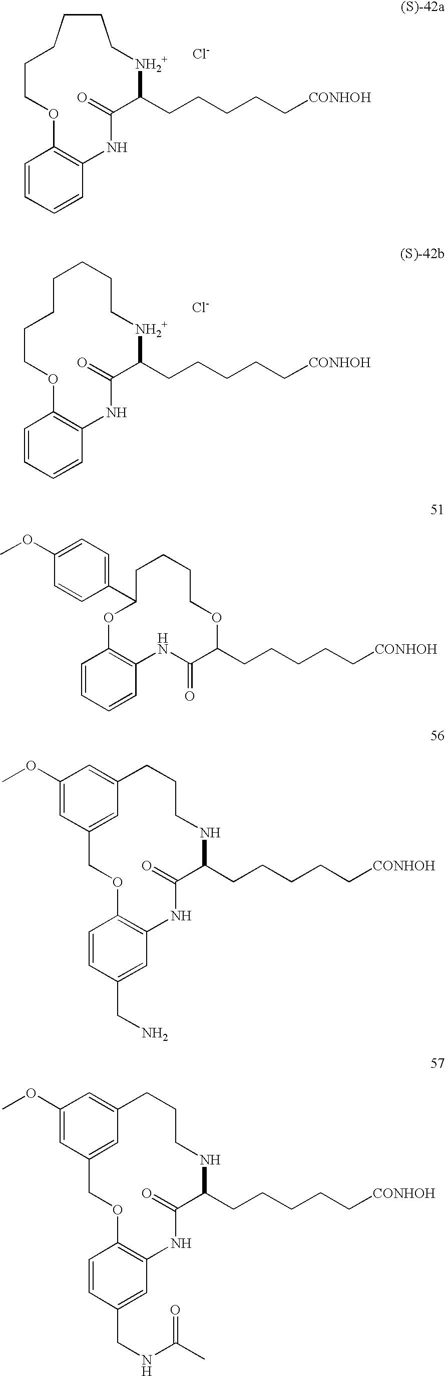

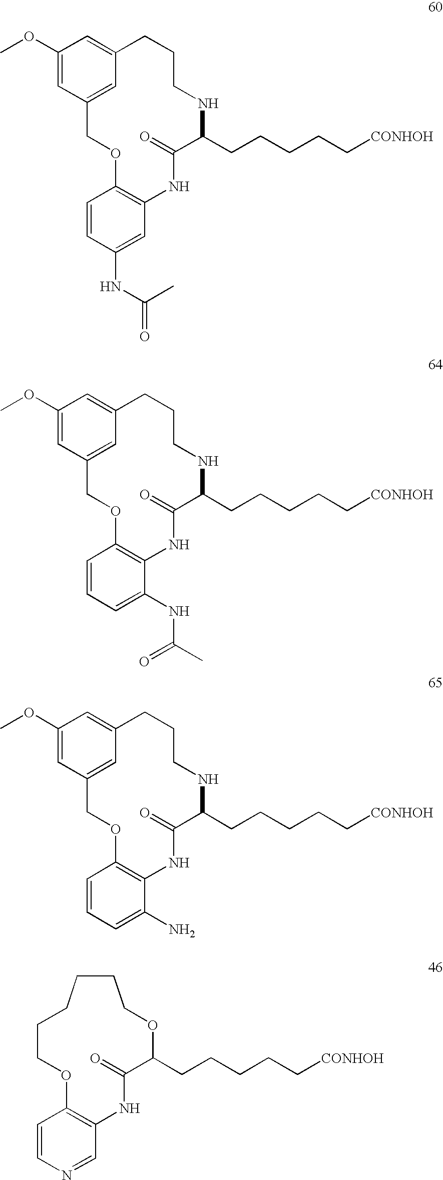

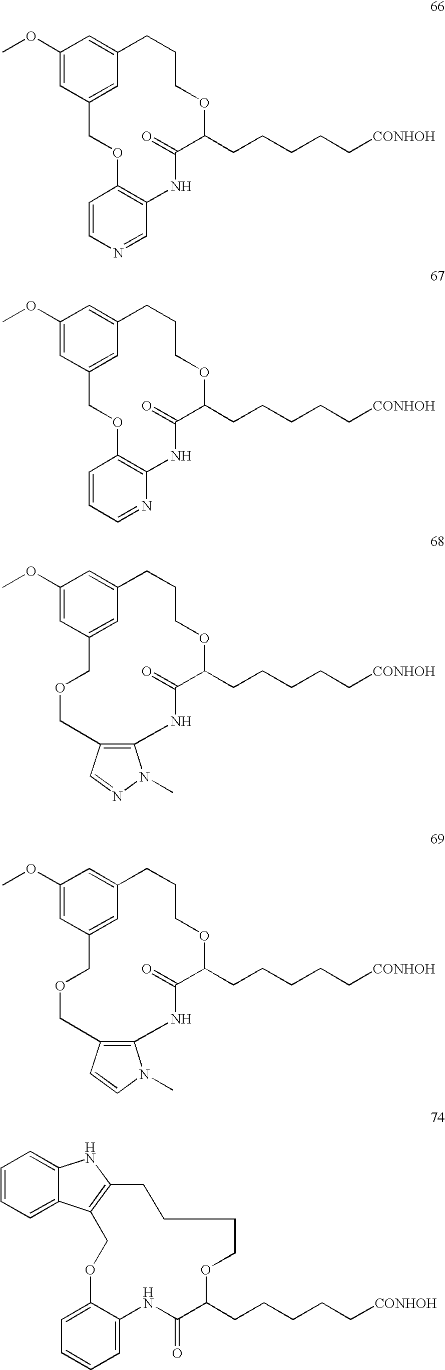

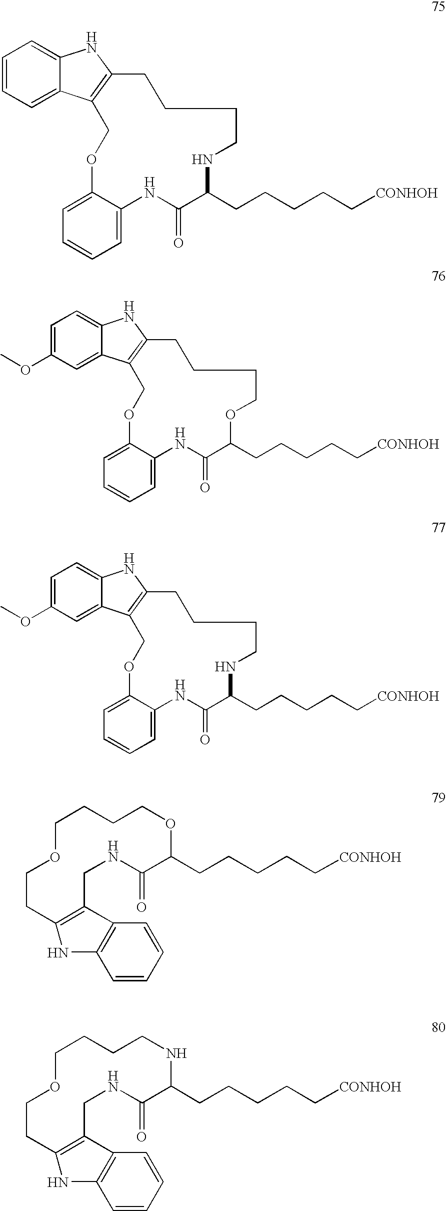

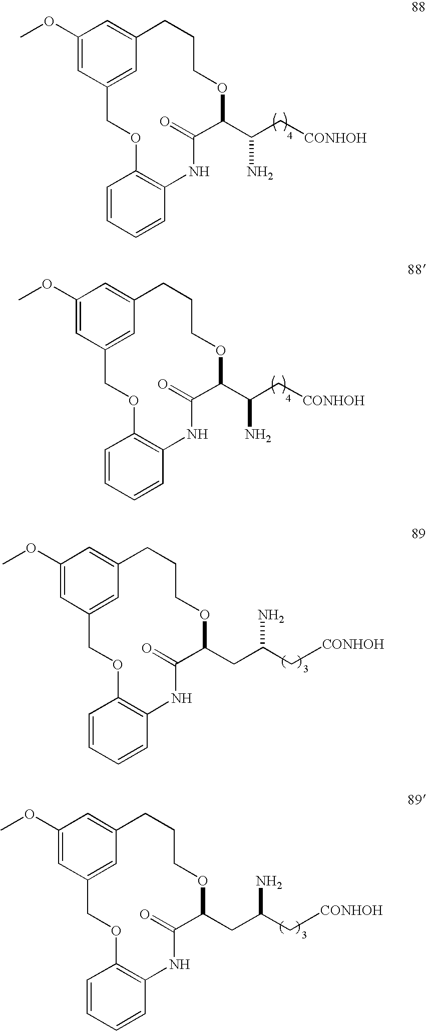

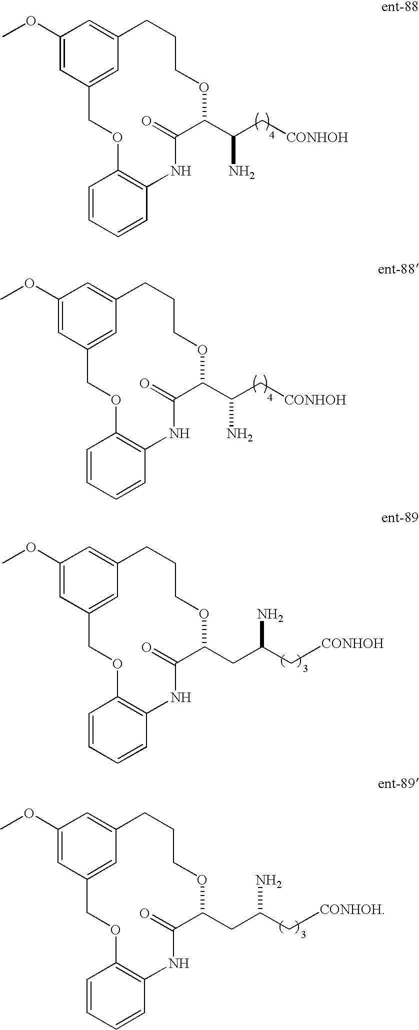

9. A compound according to claim 8, characterized in that it is selected from the group consisting of 9a, 9b, 9d, (S)-9d, (R)-9d,9e, 9f, 9g, 9h, 9j, 9k, 9l, 9m, 13d, 26b, 26c, 32, 34.

10. Pharmaceutical composition comprising a compound according to claim 1 and a pharmaceutically acceptable carrier, stabilizer, diluent or excipient thereof.

11. (canceled)

12. A method of selectively inducing terminal differentiation of neoplastic cells and thereby inhibiting proliferation of such cells, comprising administering an effective amount of a compound of claim 1 to a subject in need thereof.

13. A method of inducing differentiation of tumor cells in a tumor, comprising administering an effective amount of a compound of claim 1 to a subject having a tumor.

14. A method of inhibiting the activity of histone deacetylase, comprising administering an effective amount of a compound of claim 1 to a subject in need thereof.

15. (canceled)

16. (canceled)

17. A method of treating a primary or secondary cancer, comprising administering an effective amount of a compound of claim 1 to a subject in need thereof.
